SEQUENCE LISTING (6 sequences)

SEQ ID NO: 1 - DNA, 420 bp, Homo sapiens
  source 1..420: mol_type = genomic DNA
atggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcc

SEQ ID NO: 2 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 3 - DNA, 1323 bp, Homo sapiens
  source 1..1323: mol_type = genomic DNA
atggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagatgtgacagcacccttagtggatgagggagctcccggcaagcaggctgccgcgcagccccacacggagatcccagaaggaaccacagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttg

SEQ ID NO: 4 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 5 - DNA, 762 bp, Homo sapiens
  source 1..762: mol_type = genomic DNA
atggcgaaccttggctgctggatgctggttctctttgtggccacatggagtgacctgggcctctgcaagaagcgcccgaagcctggaggatggaacactgggggcagccgatacccggggcagggcagccctggaggcaaccgctacccacctcagggcggtggtggctgggggcagcctcatggtggtggctgggggcagcctcatggtggtggctgggggcagccccatggtggtggctggggacagcctcatggtggtggctggggtcaaggaggtggcacccacagtcagtggaacaagccgagtaagccaaaaaccaacatgaagcacatggctggtgctgcagcagctggggcagtggtggggggccttggcggctacatgctgggaagtgccatgagcaggcccatcatacatttcggcagtgactatgaggaccgttactatcgtgaaaacatgcaccgttaccccaaccaagtgtactacaggcccatggatgagtacagcaaccagaacaactttgtgcacgactgcgtcaatatcacaatcaagcagcacacggtcaccacaaccaccaagggggagaacttcaccgagaccgacgttaagatgatggagcgcgtggttgagcagatgtgtatcacccagtacgagagggaatctcaggcctattaccagagaggatcgagcatggtcctcttctcctctccacctgtgatcctcctgatctctttcctcatcttcctgatagtgggatga

SEQ ID NO: 6 - protein, 245 aa, Homo sapiens [sequence omitted]

(19) United States
(12) Patent Application Publication
(10) Pub. No.: US 2025/0230205 A1
(43) Pub. Date: Jul. 17, 2025

(54) TREATING PROTEIN MISFOLDING DISORDERS

(71) Applicants: Steven Olson, Boston, MA (US); University of Massachusetts, Boston, MA (US)

(72) Inventors: Amanda Woerman, Leverett, MA (US); Steven H. Olson, San Diego, CA (US)

(21) Appl. No.: 19/054,806

(22) Filed: Feb. 15, 2025

Related U.S. Application Data
(63) Continuation of application No. PCT/US2023/072173, filed on Aug. 14, 2023.
(60) Provisional application No. 63/371,497, filed on Aug. 15, 2022.

(51) Int. Cl. C07K 14/47; A61K 38/00; G01N 33/68

(52) U.S. Cl. CPC C07K 14/4702; G01N 33/6896; A61K 38/00; G01N 2800/28

(57) ABSTRACT

This disclosure relates to methods of treating and diagnosing protein misfolding disorders. In some embodiments, the treatment is with gene therapy, gene editing, or administration of protective proteins.

### CROSS-REFERENCE TO RELATED APPLICATION

This application is a continuation of International Application No. PCT/US2023/072173 filed on Aug. 14, 2023, which claims the benefit of priority of U.S. Provisional Application No. 63/371,497, filed Aug. 15, 2022, both of which are each incorporated by reference herein in their entireties for any purpose.

### GOVERNMENT SUPPORT

This invention was made with government support under Research Grant No. R01NS121294 awarded by NIH/National Institute of Neurological Disorders and Stroke. The government has certain rights in the invention.

### SEQUENCE LISTING

This application contains a Sequence Listing which has been submitted electronically in ST26 format and is hereby incorporated by reference in its entirety. Said ST26 file, created on Aug. 10, 2023, is named “2023 Aug. 10-01364-7001-00PCT-ST26.xml” and is 9,516 bytes in size.

### DESCRIPTION FIELD

This disclosure relates to methods of treating and diagnosing protein misfolding disorders. In some embodiments, the treatment is with gene therapy, gene editing, or administration of protective proteins.

### BACKGROUND

More than 6 million Americans suffer from a neurodegenerative disorder (NDD), but there are currently no clinically proven disease-altering therapeutics available to treat these patients. The majority of NDDs, including Alzheimer's disease (AD), Parkinson's disease (PD), and multiple system atrophy (MSA), are classified as protein misfolding disorders, which use the prion mechanism to induce progressive degeneration. For example, in MSA, the neuronal protein α-synuclein (α-syn) misfolds into a self-propagating amyloid (flat conformation), capable of acting as a templating surface for additional α-syn misfolding (this is often referred to as the prion mechanism of disease). As a result, the soluble protein is sequestered into large aggregates that continue to propagate disease throughout the brain via this prion mechanism, ultimately leading to autonomic and motor dysfunction that is invariably fatal. To interfere with the spread of proteins like α-syn and tau, which employ the same disease mechanism to cause AD, several groups have proposed knocking down protein expression to reduce the amount of substrate available for templating. However, both α-syn and tau play important roles in the neuron, raising concerns that these strategies could exacerbate neurological symptoms via loss of function in the brain.

### SUMMARY

In accordance with the description, gene therapy, gene editing, or administration of protective proteins is described herein that prevents protein misfolding. These therapeutic approaches can be applied to all NDDs that are mediated by prion protein misfolding, regardless of whether they are sporadic or familial in etiology.

Provided herein also is a diagnostic that allows for rapid identification of the neurodegenerative disease or protein misfolding disease of a patient. At the present time, a definitive diagnosis for any neurodegenerative disease is done only by autopsy, and incorrect diagnoses during early stages of disease are common.

Further provided herein is the use of disease-selective cell lines to determine which disease a patient has and the use of selective mutations in a gene therapy or gene editing approach to treat disease.

Embodiment 1. An α-syn mutant nucleic acid encoding for a α-syn mutant protein comprising one or more amino acid substitutions as compared to SEQ ID NO: 2 chosen from;
a mutation of Q79, S87, T81, Q24, T64, N65, T75, T92, and/or T81 to alanine, valine, isoleucine, leucine, methionine, glycine, proline, or phenylalanine;
a mutation of G25, G36, G41, G47, G51, G67, G73, G84, G86, and/or G93 to proline, valine, isoleucine, methionine, threonine, or tryptophan;
a mutation of T22, G31, G36, G68, T72, G73, E83, G84, and/or D98 to cysteine, alanine, glutamic acid, glutamine, isoleucine, leucine, lysine, histidine, tryptophan, valine, or tyrosine;
a mutation of H50, K45 and/or K47 to a hydrophobic and/or nonchelating amino acid such as alanine, cysteine, phenylalanine, glycine, isoleucine, lysine, leucine, methionine, asparagine, proline, glutamine, serine, threonine, valine, tryptophan, or tyrosine;
a mutation of T59, T54 and/or T72 to a nonpolar amino acid, an acidic amino acid, or a basic amino acid such as alanine, cysteine, phenylalanine, glycine, isoleucine, lysine, leucine, methionine, asparagine, proline, glutamine, valine, tryptophan, or tyrosine; and/or
an A30G, A53E, E46K, V55Y, A53E, T72M, V74I, and/or K80E, K80N, or K80W mutation.

Embodiment 2. The α-syn mutant nucleic acid of embodiment 1, wherein the substitution is K80E, K80N, or K80W.

Embodiment 3. The α-syn mutant nucleic acid of embodiment 1 or embodiment 2, wherein the nucleic acid comprises two or more, three or more, four or more, or five or more amino acid substitutions as compared to SEQ ID NO: 2.

Embodiment 4. The α-syn mutant nucleic acid of embodiment 3, wherein the two or more amino acid substitutions comprise E46K and K80E.

Embodiment 5. A tau mutant nucleic acid encoding for a tau mutant protein comprising one or more amino acid substitutions as compared to SEQ ID NO: 4 chosen from;
a G303V, K317H, D358E, N279K, G367M/V, G323F, G334F/W/A/I, G335A/I, G333A/I, G366A/I, and/or S285R mutation;
a mutation of G272, K274, V275, I277, K298, G304, S305, V306, V309, K317, N327, I328, D348, P364, N368, T377, F378, R379, E380, H388, and/or E391 to a rigid amino acids such as proline, valine, isoleucine, methionine, threonine, or tryptophan;
a mutation of Q269, K274, V275, I277, K280, K281, L284, N286, Q288, K290, S293, K294, D295, N296, K298, H299, S305, V306, Q307, 1308, V309, K311, L315, K317, C322, S324, L325, N327, I328, H329, K331, E338, K343, D345, D348, R349, L357, D358, N359, 1360, V363, N368, K370, H374, L376, R379, E380, N381, H388, E391, K395, H407, D421, A426, D430, E431, and/or A434 to amino acids to proline, valine, isoleucine, methionine, threonine or tryptophan; and/or
a mutation of G271, G272, G273, K274, I277, K281, D283, S285, N286, Q288, S289, G292, N296, K298, G302, G303, G304, S305, Y310, K317, S320, C322, G323, S324, L325, G326, I328, H330, P332, G333, G334, G335, E338, S341, E342, K347, D348, S352, I354, G355, S356, D358, H362, P364, G365, G366, G367, T377, F378, R379, E380, N381, H388, G401, A426, and/or G440 to cysteine, alanine, glutamic acid, glutamine, isoleucine, leucine, lysine, histidine, tryptophan, valine, or tyrosine.

Embodiment 6. The tau mutant nucleic acid of embodiment 5, wherein the substitution is chosen from K317H, D358E, N279K and/or S285R.

Embodiment 7. The tau mutant nucleic acid of embodiment 5 or embodiment 6, wherein the nucleic acid comprises two or more, three or more, four or more, or five or more amino acid substitutions as compared to SEQ ID NO: 4.

Embodiment 8. A mutant nucleic acid encoding for a mutant protein comprising one or more amino acid substitutions, wherein the mutant protein is:
prion protein, optionally wherein the substitution is comprised within the region of amino acids 127-219;
β-amyloid, optionally wherein the substitution is comprised within amino acids 9-42;
TDP-43;
SOD1;
HTT;
ApoE; or
TMEM106B, optionally wherein the substitution is comprised within amino acids 120-254.

Embodiment 9. A cell comprising the nucleic acid of any one of embodiments 1-8.

Embodiment 10. A mutant protein encoded by the nucleic acid of any one of embodiments 1-8.

Embodiment 11. A cell comprising a nucleic acid that expresses a mutant protein of embodiment 10.

Embodiment 12. A method to inhibit prion propagation of misfolded protein conformations (e.g., amyloids) comprising introducing one or more substitutions in the amino acid sequence of the protein so as to inhibit said protein misfolding and aggregate formation.

Embodiment 13. A method to treat a neurodegenerative disease, comprising administering an exogenous mutant protein to a subject in need thereof, wherein the neurodegenerative disease is the result of protein misfolding and aggregate formation, wherein the exogenous mutant protein comprises one or more substitutions in the amino acid sequence of the protein so as to inhibit said protein from misfolding.

Embodiment 14. A method to treat a neurodegenerative disease, comprising introducing a mutation in vivo in the gene of the protein that is misfolding in a subject in need thereof, wherein the neurodegenerative disease is the result of protein misfolding and aggregate formation, wherein the mutation results in one or more substitutions in the amino acid sequence of the protein to inhibit said protein from misfolding.

Embodiment 15. The method of any of embodiments 12-14, wherein the substitution creates a change in the charge of the protein, substitutes a larger or smaller amino acid as compared to the wild type amino acid in the same position, disrupts a salt bridge, changes conformation and/or disrupts zipper formation.

Embodiment 16. The method of any one of embodiments 12-14, wherein the substitution is the result of substituting one amino acid for a larger amino acid.

Embodiment 17. The method of any one of embodiments 12-16, wherein the mutation is introduced by gene therapy or gene editing.

Embodiment 18. The method of any one of embodiments 12-17, wherein the protein is α-syn, tau, prion protein, β-amyloid, TDP-43, SOD1, HTT, ApoE, or TMEM106B.

Embodiment 19. The method of embodiment 18, wherein the protein is α-syn mutant comprising one or more amino acid substitutions as compared to SEQ ID NO: 2 chosen from;
a mutation of Q79, S87, T81, Q24, T64, N65, T75, T92, and/or T81 to alanine, valine, isoleucine, leucine, methionine, glycine, proline, or phenylalanine;
a mutation of G25, G36, G41, G47, G51, G67, G73, G84, G86, and/or G93 to proline, valine, isoleucine, methionine, threonine, or tryptophan;
a mutation of T22, G31, G36, G68, T72, G73, E83, G84, and/or D98 to cysteine, alanine, glutamic acid, glutamine, isoleucine, leucine, lysine, histidine, tryptophan, valine, or tyrosine;
a mutation of H50, K45 and/or K47 to a hydrophobic and/or nonchelating amino acid such as alanine, cysteine, phenylalanine, glycine, isoleucine, lysine, leucine, methionine, asparagine, proline, glutamine, serine, threonine, valine, tryptophan, or tyrosine;
a mutation of T59, T54 and/or T72 to a nonpolar amino acid, an acidic amino acid, or a basic amino acid such as alanine, cysteine, phenylalanine, glycine, isoleucine, lysine, leucine, methionine, asparagine, proline, glutamine, valine, tryptophan, or tyrosine; and/or
an A30G, A53E, E46K, V55Y, A53E, T72M, V74I, and/or K80E, K80N, or K80W mutation.

Embodiment 20. The method of embodiment 19, wherein the substitution is K80E, K80N, or K80W.

Embodiment 21. The method of embodiment 19 or embodiment 20, wherein the nucleic acid comprises two or more, three or more, four or more, or five or more amino acid substitutions as compared to SEQ ID NO: 2.

Embodiment 22. The method of embodiment 21, wherein the two or more amino acid substitutions comprise E46K and K80E.

Embodiment 23. The method of embodiment 18, wherein the protein is a tau mutant comprising one or more amino acid substitutions as compared to SEQ ID NO: 4 chosen from;
a G303V, K317H, D358E, N279K, G367M/V, G323F, G334F/W/A/I, G335A/I, G333A/I, G366A/I, and/or S285R mutation;
a mutation of G272, K274, V275, I277, K298, G304, S305, V306, V309, K317, N327, I328, D348, P364, N368, T377, F378, R379, E380, H388, and/or E391 to a rigid amino acids such as proline, valine, isoleucine, methionine, threonine, or tryptophan;
a mutation of Q269, K274, V275, I277, K280, K281, L284, N286, Q288, K290, S293, K294, D295, N296, K298, H299, S305, V306, Q307, 1308, V309, K311, L315, K317, C322, S324, L325, N327, I328, H329, K331, E338, K343, D345, D348, R349, L357, D358, N359, 1360, V363, N368, K370, H374, L376, R379, E380, N381, H388, E391, K395, H407, D421, A426, D430, E431, and/or A434 to amino acids to proline, valine, isoleucine, methionine, threonine or tryptophan; and/or
a mutation of G271, G272, G273, K274, I277, K281, D283, S285, N286, Q288, S289, G292, N296, K298, G302, G303, G304, S305, Y310, K317, S320, C322, G323, S324, L325, G326, I328, H330, P332, G333, G334, G335, E338, S341, E342, K347, D348, S352, I354, G355, S356, D358, H362, P364, G365, G366, G367, T377, F378, R379, E380, N381, H388, G401, A426, and/or G440 to cysteine, alanine, glutamic acid, glutamine, isoleucine, leucine, lysine, histidine, tryptophan, valine, or tyrosine.

Embodiment 24. The method of embodiment 23, wherein the substitution is chosen from K317H, D358E, N279K and/or S285R.

Embodiment 25. The method of embodiment 23 or embodiment 24, wherein the nucleic acid comprises two or more, three or more, four or more, or five or more amino acid substitutions as compared to SEQ ID NO: 4.

Embodiment 26. A method to treat a neurodegenerative disease, comprising introducing a mutation in vivo in the gene of a protein that is misfolding in a subject in need thereof or administering an exogenous mutant protein comprising a mutation to a subject in need thereof, wherein the neurodegenerative disease is the result of protein misfolding and aggregate formation, wherein the mutation renders the subject functionally heterozygous expressing two variants of the same protein.

Embodiment 27. The method of embodiment 26, wherein the protein is the prion protein, optionally wherein the sequence of prion protein comprises SEQ ID NO: 6.

Embodiment 28. The method of embodiment 26 or embodiment 27, wherein:
the subject expresses E219, and the mutation is E219K;
the subject expresses K219, and the mutation is K219E;
the subject expresses M129, and the mutation is M129V;
the subject expresses V129, and the mutation is V129M;
the subject expresses V127, and the mutation is V127G; or
the subject expresses G127, and the mutation is G127V.

Embodiment 29. The method of any one of embodiments 26-28, wherein the mutation is introduced by gene therapy or gene editing.

Embodiment 30. The method of any one of embodiments 12-28, wherein disease symptoms become less severe as the subject expresses more protein from the gene comprising the mutation.

Embodiment 31. A method to diagnose a neurodegenerative disease comprising:
adding a sample of misfolded protein aggregate from a patient to a panel of cell lines, wherein each cell line expresses a different tagged protein of interest comprising a reporter, wherein the protein of interest contains one or more mutations; and
measuring the amount of aggregation of the reporter in each cell line comprised in the panel, wherein the tagged protein of interest expressed in each cell line that lacks aggregation of the reporter indicates the neurodegenerative disease of the patient. Embodiment 32. The method of embodiment 31, wherein:
the protein of interest comprised in one or more cell line in the panel is a mutant protein that inhibits aggregation of a protein associated with one or more neurodegenerative disease;
the protein of interest comprised in one or more cell line in the panel is a protein associated with one or more neurodegenerative disease; and/or
the method identifies the misfolding or protein aggregation underlying the patient's symptoms and/or identifies how this misfolding or protein aggregation can be inhibited.

Embodiment 33. The method of embodiment 31 or 32, wherein the tagged protein of interest expressed in multiple cell lines in the panel that lack aggregation of the reporter indicates the neurodegenerative disease of the patient.

Embodiment 34. The method of any one of embodiments 31-33, wherein one or more cell line in the panel of cell lines is a transformed cell line, optionally wherein the transformed cell line is chosen from NIH 3T3, HeLa, PC12, SH-SY5Y, or HEK293T cells.

Embodiment 35. The method of any one of embodiments 31-34, wherein the sample is obtained from a biopsy, nasal swab, skin, cerebral spinal fluid, and/or plasma samples.

Embodiment 36. The method of any one of embodiments 31-35, wherein the reporter is a fluorescent protein.

Embodiment 37. The method of any one of embodiments 31-36, further comprising treating said patient for said neurological disorder using the method of any one of embodiments 12-29.

Embodiment 38. The method of any one of embodiments 12-37, wherein the neurodegenerative disease is a:
tau-related disease, optionally wherein the tau-related disease is Alzheimer's disease, progressive supranuclear palsy, chronic traumatic encephalopathy, Pick's disease, argyrophilic grain disease, or corticobasal degeneration;
α-synuclein-related disease, optionally wherein the α-synuclein-related disease is Parkinson's disease, multiple system atrophy (MSA), Lewy body diseases, and Parkinson's disease with dementia: Huntington's disease, amyotrophic lateral sclerosis, or diabetes; or
prion protein (PrP)-related disease, optionally wherein the PrP-related disease is Creutzfeldt-Jakob disease, fatal familial insomnia, or Gerstmann-Sträussler-Scheinker disease.

In addition to the embodiments above, also disclosed herein are a number of items.

Item 1. An α-syn mutant nucleic acid encoding for a α-syn mutant protein comprising one or more amino acid substitutions selected from the group consisting of K80E, K80N, and K80W.

Item 2. An α-syn mutant protein comprising one or more amino acid substitutions selected from the group consisting of K80E, K80N, and K80W. Item 3. A tau mutant nucleic acid encoding for a tau mutant protein comprising one or more amino acid substitutions selected from the group consisting of K317H, D358, N279K and S285R.

Item 4. A tau mutant protein comprising one or more amino acid substitutions selected from the group consisting of K317H, D358, N279K and S285R.

Item 5. A cell comprising a nucleic acid that expresses an α-syn mutant, wherein the α-syn mutant comprises one or more amino acid substitutions selected from the group consisting of K80E, K80N, and K80W.

Item 6. A cell comprising a nucleic acid that expresses a tau mutant, wherein the tau mutant comprises one or more amino acid substitutions selected from the group consisting of K317H, D358, N279K and S285R.

Item 7. A method to inhibit the prion propagation of misfolded protein conformations (e.g., amyloids) comprising introducing one or more substitutions in the amino acid sequence of the protein so as to inhibit said protein misfolding or aggregate protein formations.

Item 8. The method of item 7, wherein the protein is a α-syn or tau mutant.

Item 9. The method of item 7 or 8, wherein the substitution creates a change in the charge of the protein, substitutes a larger or smaller amino acid as compared to the wild type amino acid in the same position, disrupts a salt bridge, changes conformation and/or disrupts zipper formation.

Item 10. The method of item 9, wherein the substitution is the result of substituting one amino acid for a larger amino acid.

Item 11. The method of item 8, wherein the α-syn mutant comprises one or more amino acid substitutions selected from the group consisting of K80E, K80N, and K80W.

Item 12. The method of item 8, wherein the tau mutant comprises one or more amino acid substitutions selected from the group consisting of K317H, D358, N279K and S285R.

Item 13. A method to treat a neurodegenerative disease, comprising administering an exogenous mutant protein to a subject in need thereof, wherein the neurodegenerative disease is the result of protein misfolding or aggregate protein formation, wherein the exogenous mutant protein comprises one or more substitutions in the amino acid sequence of the protein so as to inhibit said protein from misfolding.

Item 14. A method to treat a neurodegenerative disease, comprising introducing a mutation in vivo in the gene of the protein that is misfolding in a subject in need thereof, wherein the neurodegenerative disease is the result of protein misfolding or aggregate protein formation, wherein the mutation results in one or more substitutions in the amino acid sequence of the protein to inhibit said protein from misfolding.

Item 15. The method of item 13 or 14, wherein the protein is a α-syn or tau mutant.

Item 16. The method of any of items 13 to 15, wherein the substitution creates a change in the charge of the protein, substitutes a larger or smaller amino acid as compared to the wild type amino acid in the same position, disrupts a salt bridge, changes conformation and/or disrupts zipper formation.

Item 17. The method of any one of items 13 to 16, wherein the substitution is the result of substituting one amino acid for a larger amino acid.

Item 18. The method of item 15, wherein the α-syn mutant comprises one or more amino acid substitutions selected from the group consisting of K80E, K80N, and K80W.

Item 19. The method of item 15, wherein the tau mutant comprises one or more amino acid substitutions selected from the group consisting of K317H, D358, N279K and S285R.

Item 20. The method of any one of items 10 to 16, wherein the neurodegenerative disease comprises tau-related diseases (or tauopathies), including, but not limited to, Alzheimer's disease (in which the proteins AB, ApoE, and TMEM106B also contribute to the disease and may be targeted using the same approach), progressive supranuclear palsy, chronic traumatic encephalopathy, Pick's disease, argyrophilic grain disease, and corticobasal degeneration: α-synuclein-related diseases (or synucleinopathies), including, but not limited to, Parkinson's disease, multiple system atrophy (MSA), Lewy body diseases, and Parkinson's disease with dementia: Huntington's disease (caused by mutant Htt), amyotrophic lateral sclerosis (ALS: caused by TDP-43 and/or SOD1), diabetes, prion protein (PrP)-related diseases, including but not limited to, Creutzfeldt-Jakob disease, fatal familial insomnia, and Gerstmann-Sträussler-Scheinker disease.

Item 21. A method to diagnose a neurodegenerative disease comprising:
adding a sample of misfolded protein aggregate from a patient to a collection of cell lines, wherein each cell line expresses a different protein of interest tagged with a reporter, wherein the protein of interest contains one or more specific mutations;
wherein the protein of interest in the cell line that prevents aggregation of the tagged protein indicates the specific neurodegenerative disease of the patient.

Item 22. The method of item 21, wherein the sample is obtained from a biopsy, nasal swab, skin, cerebral spinal fluid, and/or plasma samples.

Item 23. The method of item 21, wherein the reporter is a fluorescent protein.

Item 25. The method of any one of items 21-23, further comprising treating said patient for said neurological disorder.

Additional objects and advantages will be set forth in part in the description which follows, and in part will be understood from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate one or more embodiment(s) and together with the description, serve to explain the principles described herein.

### BRIEF DESCRIPTION OF THE DRAWINGS

FIG. 1 depicts monomeric α-synuclein and misfolded α-synuclein that leads to disease states, such as MSA and PD.

FIGS. 2A-2B show that point mutations interfere with protein misfolding in NDD. (A) α-Syn aggregates isolated from MSA patient samples propagate in α-syn140-YFP cells, but not when the E46K, K80E, or K80W mutations are present. (B) Tau aggregates isolated from Tg (MAPT*P301S^{+/+}) mice propagate in tauRD-YFP cells expressing the P301W and G303V mutations, but not in the presence of N279K or S285R mutations.

FIGS. 3A-3C show two possible experimental outcomes in an MSA model of disease. (A) MSA templates using WT α-syn to propagate disease. Under normal/disease conditions MSA recruits soluble WT α-syn to template misfolding, which may cause progressive degeneration. (B) Gene editing to create conversion-incompetent α-syn will interfere with the templating process. For example, use of CRISPR prime editing to convert WT α-syn to contain a mutation that blocks MSA propagation. (C) Mutant α-syn may use in trans dominant-negative templating to halt disease progression. For example, use of an AAV to deliver an exogenous copy of the mutant gene to cause dominant negative inhibition of MSA propagation.

FIGS. 4A-4D show results in Tau model. Tau aggregates isolated from corticobasal degeneration (CBD), but not progressive supranuclear palsy (PSP), patient samples propagate in Tau4RD-YFP cells expressing the (A) N279K and (B) S285R mutations. Both CBD and PSP showed robust propagation in cells expressing the (C) P301W mutation, and reduced propagation in the (D) G303V cell line. P values comparing CBD and PSP: N279K, 0.02; S285R, 0.11; P301W, 1.00; and G303V, 0.16.

FIGS. 5A-5F show that MSA patient samples transmit neurological disease to TgM20^{+/-} mice. Eight-week-old TgM20^{+/-} mice were inoculated with either 30 μL of two control patient samples (C9 and C17) or six MSA patient samples (MSA5, MSA6, MSA13, MSA14, MSA16, and MSA17). Kaplan-Meier plots show disease onset in female (solid line) and male (dotted line) TgM20^{+/-} mice for MSA5 (A), MSA6 (B), MSA13 (C), MSA14 (D), MSA 16(E), and MSA17 (F). Control-inoculated mice (C9 and C17) did not develop disease by 475 days postinoculation (dpi), but mice inoculated with all six MSA samples developed progressive neurological signs. Incubation times shown in Table 4 (**=P<0.01; ***=P<0.001; ****=P<0.0001). Female mice inoculated with MSA patient samples developed neurological disease ˜63 days earlier than male mice (P=0.0005).

FIGS. 6A and 6B show that MSA patient samples induce phosphorylated α-synuclein neuropathology in terminal TgM20^{+/-} mice. Eight-week-old TgM20^{+/-} mice were inoculated with 30 μL of C9 control sample or six MSA patient samples (MSA5, MSA6, MSA13, MSA14, MSA16, and MSA17). (A) Quantification of stained brain slices showed no phosphorylated α-synuclein inclusions were present in the caudate (Cd), hippocampus (HC), piriform cortex and amygdala (PC), thalamus (Thal), hypothalamus (HTH), midbrain (Mid), or pons of C9-inoculated mice. However, the MSA patient samples induced neuropathological lesions in the HTH, Mid, and pons (*=P<0.05; **=P<0.01; ***=P<0.001; ****=P<0.0001). (B) Representative images of the Mid from TgM20^{+/-} mice inoculated with C9, MSA5, MSA6, or MSA17 brain homogenate. MSA-inoculated mice developed neuronal inclusions containing phosphorylated α-synuclein (pSyn), as well as reactive astrogliosis (GFAP), whereas control-inoculated mice did not. Only cell nuclei were detectable via DAPI. Scale bar: 50 μm.

FIG. 7 shows that MSA patient samples transmit α-synuclein prions to TgM20^{+/-} mice. Eight-week-old TgM20^{+/-} mice were inoculated with either 30 μL of two control patient samples (C9 and C17) or six MSA patient samples (MSA5, MSA6, MSA13, MSA14, MSA16, and MSA17). Brain homogenates from inoculated TgM20^{+/-} mice were assessed for α-synuclein prion transmission using a cell-based assay [36, 40]. α-Synuclein prions were isolated via phosphotungstic acid precipitation and were incubated with α-syn-YFP cells for 4 d. The cell lines tested express wild-type (WT) α-syn-YFP, single mutations (E46K and A53T), double mutations (A30P,A53T and E46K,A53T), or A53T truncated at residue 95 (1-95). None of the homogenates from C9-inoculated TgM20^{+/-} mice infected the cells, but MSA-inoculated mouse samples infected WT, A53T, and A30P,A53T cells (*=P<0.05: ***=P<0.001: ****=P<0.0001).

FIG. 8 shows that recombinant WT PFFs induce neurological disease in TgM20^{+/-} mice. Eight-week-old TgM20^{+/-} mice were inoculated with either control patient sample (C9 and C17) or recombinant WT PFFs. Kaplan-Meier plot of disease onset in TgM20^{+/-} mice shows control-inoculated mice did not develop disease by 475 days postinoculation (dpi), but WT PFF-inoculated mice developed overt motor deficits 210±37 dpi (***=P<0.001).

FIGS. 9A-9C show that WT PFFs induce robust phosphorylated α-synuclein pathology in terminal TgM20^{+/-} mice. Eight-week-old TgM20^{+/-} mice were inoculated with either 30 μL of two control patient samples (C9 and C17) or 30 μg of WT PFFs. (A) Quantification of stained brain slices showed no phosphorylated α-synuclein inclusions were present in the caudate (Cd), hippocampus (HC), piriform cortex and amygdala (PC), thalamus (Thal), hypothalamus (HTH), midbrain (Mid), or pons of control-inoculated mice. However, WT PFFs induced robust neuropathological lesions in the HC, Thal, HTH, Mid, and pons (*=P<0.05: ***=P<0.001: ****=P<0.0001). (B and C) Representative images from either C9-inoculated (top row) or WT PFF-inoculated mice (bottom row). WT PFF-inoculated mice developed neuronal pSyn inclusions and astrogliosis (GFAP), whereas control-inoculated animals did not. (B) Images on the left include the HC, PC, Thal, and HTH. Images on the right include the pons and a portion of the cerebellum. Scale bar: 500 μm. (C) Higher magnification of Mid α-synuclein neuropathology. Scale bar: 50 μm.

FIG. 10 shows that WT PFFs induce α-synuclein prion formation in TgM20^{+/-} mice. Eight-week-old TgM20^{+/-} mice were inoculated with either control patient sample (C9 and C17) or recombinant WT PFFs. Brain homogenates were assayed for α-synuclein prion transmission using a cell-based assay [36, 40]. α-Synuclein prions were isolated from brain homogenates via phosphotungstic acid precipitation and were incubated with α-syn-YFP cells for 4 d. The cell lines tested express wild-type (WT) α-syn-YFP, single mutations (E46K and A53T), double mutations (A30P,A53T and E46K,A53T), or A53T truncated at residue 95 (1-95). None of the homogenates from control-inoculated TgM20^{+/-} mice infected the cells, but WT PFF-inoculated mouse samples infected WT, A53T, A30P,A53T, and 1-95 cells (****=P<0.0001).

FIGS. 11A and 11B show that MSA and WT PFFs contain distinct α-synuclein prion strains. Eight-week-old TgM20^{+/-} mice were inoculated with either MSA patient samples or recombinant WT PFFs. Measurements from MSA-inoculated mice are averaged across experiments testing all 6 MSA patient samples. (A) Quantification of stained brain slices showed a significant increase in phosphorylated α-synuclein lesions in the thalamus (Thal), hypothalamus (HTH), and midbrain (Mid). Cd, caudate; HC, hippocampus; and PC, piriform cortex, and amygdala (*=P<0.05; ****=P<0.0001). (B) Brain homogenates were assayed for α-synuclein prion transmission using a cell-based assay [36, 40]. α-Synuclein prions were isolated from brain homogenates via phosphotungstic acid precipitation and were incubated with α-syn-YFP cells for 4 days. The cell lines tested express wild-type (WT) α-syn-YFP, single mutations (E46K and A53T), double mutations (A30P,A53T and E46K,A53T), or A53T truncated at residue 95 (1-95). WT PFF-inoculated mouse samples showed greater infectivity in the A30P, A53T and 1-95 cell lines than the MSA-inoculated samples (****=P<0.0001).

FIGS. 12A-12C show that hemizygous TgM20^{+/-} mice do not develop spontaneous disease. (A) TgM20^{+/-} mice were aged to 546 days (˜18 months) and were assessed for neurological signs twice each week. Consistent with previously reported studies [Giasson et al. (2002) Neuron 34:521-533], hemizygous TgM20^{+/-} mice did not develop spontaneous disease. (B) Frozen half-brains from TgM20^{+/-} mice collected at 546 days of age were homogenized and α-synuclein aggregates were isolated by phosphotungstic acid precipitation. Protein pellets were incubated with α-syn140-YFP (WT), α-syn140*E46K-YFP (E46K), α-syn140*A53T-YFP (A53T), α-syn140*A30P,A53T-YFP (A30P,A53T), α-syn140*E46K,A53T-YFP (E46K,A53T), and α-syn1-95*A53T-YFP (1-95) cells to measure α-synuclein prion infectivity. None of the mice tested developed detectable spontaneous α-synuclein prions. (C) Formalin-fixed half-brains from TgM20^{+/-} mice collected at 546 days of age were immunostained for phosphorylated α-synuclein. Neuropathology was quantified (percent area) in the caudate (Cd), hippocampus (HC), piriform cortex and amygdala (PC), thalamus (Thal), hypothalamus (HTH), midbrain (Mid), and pons. None of the aged mice developed spontaneous α-synuclein pathology.

FIGS. 13A and 13B show that MSA patient samples and WT PFFs induce the formation of detergent-insoluble α-synuclein aggregates. Eight-week-old TgM20^{+/-} mice were inoculated with control patient sample and either (a) MSA patient samples or (b) WT PFFs. Brain homogenates from asymptomatic control mice euthanized 475 days postinoculation (dpi) or symptomatic MSA-inoculated or WT PFF-inoculated mice were analyzed for the presence of sarkosyl-insoluble α-synuclein aggregates. (A) Two sets of Western blots tested eight mice each. Each blot contains one sample from each inoculation group (two control and six MSA). No phosphorylated insoluble α-synuclein was detected in control-inoculated mice, but it was present in MSA-inoculated animals. (B) Two brain homogenates from control-inoculated TgM20^{+/-} mice and three homogenates from WT PFF-inoculated animals were assayed. No insoluble phosphorylated α-synuclein was detected in control-inoculated mice, but it was present in terminal mice inoculated with WT PFFs. For all blots, top and middle panels probed with EP1536Y primary antibody. The top panel is sarkosyl-insoluble protein, middle is soluble protein. Bottom panel shows vinculin as a loading control for the soluble blot.

FIGS. 14A-14C show that TgM47^{+/-} mice are resistant to MSA prion transmission. Eight-week-old TgM47^{+/-} mice were inoculated with either control (C9 and C17) or MSA patient samples (MSA5, MSA14, and MSA17). (A) Kaplan-Meier plot of disease onset in TgM47^{+/-} mice following inoculation. TgM47^{+/-} mice did not develop overt motor signs by 475 days postinoculation (dpi: P>0.05). (B) Two brain homogenates from TgM47^{+/-} mice inoculated with either control or with each of the MSA patient samples were assayed for the presence of insoluble α-synuclein following detergent extraction. An MSA-inoculated TgM83^{+/-} mouse brain, which has previously been shown is positive for phosphorylated α-synuclein, was included as a positive control (EP1536Y primary antibody). None of the homogenates from the inoculated mice contained aggregated α-synuclein, though total α-synuclein levels (MJFR1 primary antibody) were consistent across all homogenates tested. Vinculin is shown as a loading control. (C) Fixed half-brains from the same mice were analyzed for phosphorylated α-synuclein neuropathology in the caudate (Cd), hippocampus (HC), piriform cortex and amygdala (Pir), thalamus (Thal), hypothalamus (HTH), midbrain (Mid), and pons. None of the inoculated animals developed detectable pathology (P>0.05).

FIGS. 15A-15D show that E46K PFFs transmit neurological disease to TgM47^{+/-} mice. Eight-week-old TgM47^{+/-} mice were inoculated with 30 μg of E46K α-synuclein PFFs or 30 μL of 1% brain homogenate from control patient samples. (A) Kaplan-Meier plot shows E46K PFFs induced neurological disease 286±122 days postinoculation (dpi: ****P<0.0001). (B) Two brain homogenates from TgM47^{+/-} mice inoculated with control patient samples and four homogenates from mice inoculated with E46K PFFs were assayed for the presence of insoluble α-synuclein following detergent extraction. All four homogenates from E46K PFF-inoculated mice contained phosphorylated α-synuclein (EP1536Y primary antibody). All mice tested expressed similar levels of total α-synuclein (MJFR1 primary antibody). Vinculin is shown as a loading control. (C) Guanidine hydrochloride (GdnHCl) denaturation was performed using brain homogenates from two control-inoculated (left) and two E46K PFF-inoculated TgM47^{+/-} mice (right) using 0, 1, 2, 3, 4, and 5 M GdnHCl. After detergent extraction, α-synuclein in the resulting protein pellet was detected via Western blot with the total α-synuclein antibody MJFR1. (D) Proteinase K (PK) digestion was performed using brain homogenates from two control-inoculated (left) and two E46K PFF-inoculated TgM47^{+/-} mice (right) using 0, 0.5, 1, 2.5, and 5 μg PK. After detergent extraction, total α-synuclein in the resulting protein pellets was detected via Western blot with the antibody MJFR1.

FIGS. 16A-16B show that E46K PFFs induce phosphorylated α-synuclein neuropathology in TgM47^{+/-} mice. Fixed tissues from TgM47^{+/-} mice inoculated with the control patient sample C17 or E46K PFFs were analyzed for phosphorylated α-synuclein inclusions (EP1536Y primary antibody). (A) Quantification of neuropathology showed the caudate (Cd) was unaffected by E46K PFF transmission. The hippocampus (HC), piriform cortex and amygdala (Pir), and thalamus (Thal) developed inclusions that were not statistically different from control (P>0.05). However, E46K PFFs induced robust inclusion formation in the hypothalamus (HTH: ***P<0.001), midbrain (Mid: ****P<0.0001), and pons (****P<0.0001). (B) Representative images from TgM47^{+/-} mice inoculated with control patient samples (left column) or E46K PFFs (middle column) as well as an MSA-inoculated TgM83^{+/-} mouse sample shown for comparison (right column). From top to bottom, images of the dentate gyrus, periaqueductal gray, and pons are shown. Region denoted with a white box in the top pons row is shown at higher magnification in the bottom row. Mice inoculated with E46K PFFs developed perinuclear phosphorylated α-synuclein (pSyn) inclusions in neurons. Affected neurons were surrounded by reactive astrogliosis (GFAP). Control-inoculated mice did not develop these pathological changes: only nuclei were observed via DAPI when staining. Scale bar: 50 μm.

FIGS. 17A-17D show that TgM47^{+/-} mice propagate WT α-synuclein PFFs. Eight-week-old TgM47^{+/-} mice were inoculated with 30 μg of WT human α-synuclein PFFs. (A) Kaplan-Meier plot shows mice inoculated with WT PFFs did not develop neurological disease 531 days postinoculation (dpi: P>0.05). (B) Two brain homogenates from TgM47^{+/-} mice inoculated with control patient samples and four homogenates from mice inoculated with WT PFFs were assayed for the presence of insoluble α-synuclein following detergent extraction. An MSA-inoculated TgM83^{+/-} mouse brain, which has previously been shown is positive for phosphorylated α-synuclein, was included as a positive control (EP1536Y primary antibody). None of the samples from inoculated TgM47^{+/-} mice contained aggregated α-synuclein, though total α-synuclein levels (MJFR1 primary antibody) were consistent across all mice tested. Vinculin is shown as a loading control. (C) Guanidine hydrochloride (GdnHCl) denaturation was performed using brain homogenates from two control-inoculated (left) and two WT PFF-inoculated TgM47^{+/-} mice (right) using 0, 1, 2, 3, 4, and 5 M GdnHCl. After detergent extraction, α-synuclein in the resulting protein pellet was detected via Western blot with the total α-synuclein antibody MJFR1. (D) Proteinase K (PK) digestion was performed using brain homogenates from one control-inoculated (top) and one WT PFF-inoculated TgM47^{+/-} mouse (bottom) using 0, 0.5, 1, 2.5, and 5 μg PK. After detergent extraction, total α-synuclein in the resulting protein pellets was detected via Western blot with the antibody MJFR1.

FIGS. 18A-18C show that TgM20^{+/-} mice propagate E46K α-synuclein PFFs. Eight-week-old TgM20^{+/-} mice were inoculated with 30 μg of E46K human α-synuclein PFFs. (A) Kaplan-Meier plot shows five of eight mice inoculated with E46K PFFs developed neurological disease by 475 days postinoculation (dpi: P>0.05). Incubation times for control-inoculated mice previously reported in Holec, et al., 2022 [17]. (B) Two brain homogenates from TgM20^{+/-} mice inoculated with control patient samples and three homogenates from mice inoculated with E46K PFFs were assayed for the presence of insoluble α-synuclein following detergent extraction (EP1536Y primary antibody). Some of the samples from inoculated TgM20^{+/-} mice contained aggregated α-synuclein, though soluble α-synuclein (EP1536Y primary antibody) was present in all mice tested. Vinculin is shown as a loading control. (C) Proteinase K (PK) digestion was performed using brain homogenates from control-inoculated and E46K PFF-inoculated TgM20^{+/-} mice using 0, 0.5, 1, 2.5, and 5 μg PK. After detergent extraction, phosphorylated and total α-synuclein in the resulting protein pellets were detected via Western blot using both EP1536Y (left) and MJFR1 (right) primary antibodies, respectively.

FIGS. 19A-19B show that TgM20^{+/-} mice inoculated with E46K PFFs develop mild phosphorylated α-synuclein neuropathology. Fixed tissues from TgM20^{+/-} mice inoculated with control patient sample C17 or E46K PFFs were immunostained for phosphorylated α-synuclein (EP1536Y primary antibody) and glial fibrillary acidic protein (GFAP: astrocytes). (A) Quantification of stained brain slices showed no significant phosphorylated α-synuclein inclusions were present in the caudate (Cd), hippocampus (HC), piriform cortex and amygdala (Pir), thalamus (Thal), hypothalamus (HTH), midbrain (Mid), or pons (P>0.05). Neuropathology data for control-inoculated mice was previously reported in Holec, et al., 2022 [17]. (B) Representative images of the HC, amygdala, and pons from mice inoculated with either control patient sample C17 (top) or E46K PFFs (bottom). Notably, some E46K PFF-inoculated mice did develop some pSyn inclusions in the HC and amygdala. While these inclusions appeared to be mostly neuronal, overlap with GFAP staining suggests there may also be colocalization with astrocytes. Similar inclusions were not detected in control-inoculated mice. Scale bar: 50 μm.

FIGS. 20A-20C show that hemizygous TgM47^{+/-} mice do not develop spontaneous disease. (A) TgM47^{+/-} mice were aged to 545 days (˜18 months) and were assessed for neurological signs twice each week. However, as shown in the Kaplan-Meier plot, the mice did not develop the signs reported in homozygous TgM47^{+/+} mice (Emmer K L et al. J Biol Chem. 2011:286:35104-18). (B) Formalin-fixed half-brains from TgM47^{+/-} mice collected at 545 days of age were immunostained for phosphorylated α-synuclein. Neuropathology was quantified (percent area) in the caudate (Cd), hippocampus (HC), piriform cortex and amygdala (Pir), thalamus (Thal), hypothalamus (HTH), midbrain (Mid), and pons. None of the aged mice developed spontaneous α-synuclein pathology. (C) Representative image showing the lack of phosphorylated α-synuclein pathology in the pons of an aged mouse. No phosphorylated α-synuclein (EP1536Y primary antibody) or glial fibrillary acidic protein (GFAP) for reactive astrocytes were detected. Only nuclei stained using DAPI were observed. Scale bar: 50 μm.

FIGS. 21A and 21B show that MSA prions do not induce neuropathological inclusions in TgM47^{+/-} mice. Fixed half-brains from TgM47^{+/-} mice inoculated with either control (C9 or C17) or MSA (MSA5, MSA14, or MSA17) patient samples were collected 475 days postinoculation. Sections were immunostained using the EP1536Y (phosphorylated α-synuclein) and GFAP primary antibodies. Neither the (A) control-nor the (B) MSA-inoculated mice developed α-synuclein inclusions or signs of an immune response in the brain. Scale bar: 50 μm.

FIGS. 22A and 22B show that TgM47^{+/-} mice inoculated with wild-type (WT) preformed fibrils (PFFs) do not develop phosphorylated α-synuclein neuropathology. Fixed tissues from TgM47^{+/-} mice inoculated with a control patient sample (C17) collected 475 days postinoculation (dpi) or WT PFFs collected 531 dpi were immunostained for phosphorylated α-synuclein (EP1536Y primary antibody) and GFAP. (A) Quantification of stained brain slices showed no phosphorylated α-synuclein inclusions were present in the caudate (Cd), hippocampus (HC), piriform cortex and amygdala (Pir), thalamus (Thal), hypothalamus (HTH), midbrain (Mid), or pons. (B) Representative images of the pons from mice inoculated with either control patient sample C17 (left) or WT PFFs (right). No phosphorylated α-synuclein or GFAP staining was detected. Scale bar: 50 μm.

FIGS. 23A-23D show results of MSA mutations tested for their ability to infect or replication in α-syn-YFP cell lines. Familial mutations A30G (A) and A53E (B) blocked MSA replication. Additionally, the novel mutations V55Y (C) and V74I (D) also prevented MSA infection.

FIGS. 24A-24E show results with various K80 substitutions tested for their ability to infect or replicate in α-syn-YFP cell lines. K80E (A), K80N (B), K80Q (C), and K80W (D) substitutions all significantly interfered with MSA replication. Cells expressing the double E46K,K80E mutation (E) were also resistant to MSA replication.

FIGS. 25A-25E show results with various tau substitutions tested for their ability to infect or replicate in HEK293T cells expressing the Tau4RD construct fused to YFP. The N279K (A), G303V (C), K317H (D), and D358E mutations (E) inhibited or reduced PSP replication with little to no effect on the CBD patient samples. The S285R mutation (B) interfered with both CBD and PSP replication.

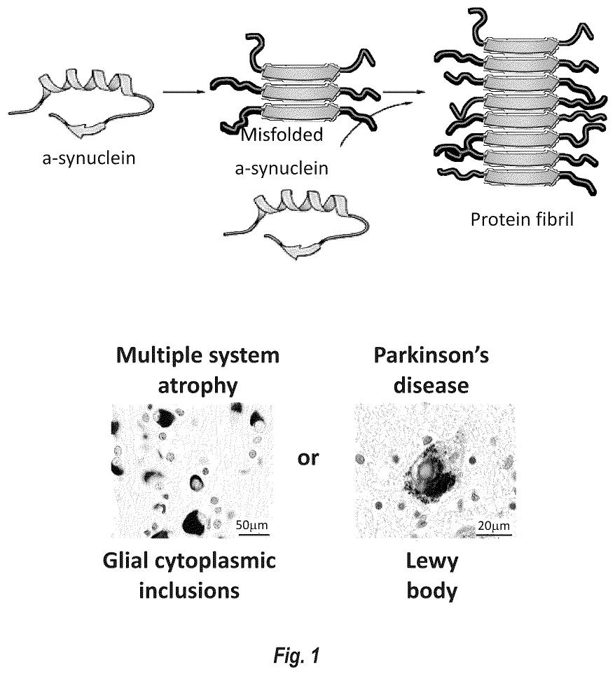

### DESCRIPTION OF THE SEQUENCES

TABLE 1
Table 1 provides a listing of certain sequences referenced herein.
Description of the Sequences

| | | SEQ |
|---|---|---|
| | | ID |
| Description | Sequences | NO |
| DNA sequence | ATGGATGTATTCATGAAAGGACTTTCAAAGGCCAAGGAG | 1 |
| for WT a- | GGAGTTGTGGCTGCTGCTGAGAAAACCAAACAGGGTGTG | |
| synuclein with | GCAGAAGCAGCAGGAAAGACAAAAGAGGGTGTTCTCTAT | |
| E46 and K80 | GTAGGCTCCAAAACCAAGGAGGGAGTGGTGCATGGTGTG | |
| bolded | GCAACAGTGGCTGAGAAGACCAAAGAGCAAGTGACAAAT | |
| | GTTGGAGGAGCAGTGGTGACGGGTGTGACAGCAGTAGC | |
| | CCAGAAGACAGTGGAGGGAGCAGGGAGCATTGCAGCAG | |
| | CCACTGGCTTTGTCAAAAAGGACCAGTTGGGCAAGAATG | |
| | AAGAAGGAGCCCCACAGGAAGGAATTCTGGAAGATATGC | |
| | CTGTGGATCCTGACAATGAGGCTTATGAAATGCCTTCTG | |
| | AGGAAGGGTATCAAGACTACGAACCTGAAGCC | |
| Amino acid | MDVFMKGLSKAKEGVVAAAEKTKQGVAEAAGKTKEGVLY | 2 |
| sequence for WT | VGSKTKEGVVHGVATVAEKTKEQVTNVGGAVVTGVTAVA | |
| a-synuclein with | QKTVEGAGSIAAATGFVKKDQLGKNEEGAPQEGILEDMP | |
| E46 and K80 | VDPDNEAYEMPSEEGYQDYEPEA | |
| bolded | | |
| DNA sequence | ATGGCTGAGCCCCGCCAGGAGTTCGAAGTGATGGAAGAT | 3 |
| for the 2N4R | CACGCTGGGACGTACGGGTTGGGGGACAGGAAAGATCAG | |
| isoform of human | GGGGGCTACACCATGCACCAAGACCAAGAGGGTGACACG | |
| tau with 279, 285, | GACGCTGGCCTGAAAGAATCTCCCCTGCAGACCCCCACT | |
| 301, 303, 317, | GAGGACGGATCTGAGGAACCGGGCTCTGAAACCTCTGAT | |
| and 358 bolded | GCTAAGAGCACTCCAACAGCGGAAGATGTGACAGCACCC | |
| | TTAGTGGATGAGGGAGCTCCCGGCAAGCAGGCTGCCGCG | |
| | CAGCCCCACACGGAGATCCCAGAAGGAACCACAGCTGAA | |
| | GAAGCAGGCATTGGAGACACCCCCAGCCTGGAAGACGAA | |
| | GCTGCTGGTCACGTGACCCAAGCTCGCATGGTCAGTAAA | |
| | AGCAAAGACGGGACTGGAAGCGATGACAAAAAAGCCAA | |
| | GGGGGCTGATGGTAAAACGAAGATCGCCACACCGCGGGG | |
| | AGCAGCCCCTCCAGGCCAGAAGGGCCAGGCCAACGCCAC | |
| | CAGGATTCCAGCAAAAACCCCGCCCGCTCCAAAGACACC | |
| | ACCCAGCTCTGGTGAACCTCCAAAATCAGGGGATCGCAG | |
| | CGGCTACAGCAGCCCCGGCTCCCCAGGCACTCCCGGCAG | |
| | CCGCTCCCGCACCCCGTCCCTTCCAACCCCACCCACCCGG | |
| | GAGCCCAAGAAGGTGGCAGTGGTCCGTACTCCACCCAAG | |
| | TCGCCGTCTTCCGCCAAGAGCCGCCTGCAGACAGCCCCC | |
| | GTGCCCATGCCAGACCTGAAGAATGTCAAGTCCAAGATC | |
| | GGCTCCACTGAGAACCTGAAGCACCAGCCGGGAGGCGGG | |
| | AAGGTGCAGATAATTAATAAGAAGCTGGATCTTAGCAAC | |
| | GTCCAGTCCAAGTGTGGCTCAAAGGATAATATCAAACAC | |
| | GTCCCGGGAGGCGGCAGTGTGCAAATAGTCTACAAACC | |
| | AGTTGACCTGAGCAAGGTGACCTCCAAGTGTGGCTCATT | |
| | AGGCAACATCCATCATAAACCAGGAGGTGGCCAGGTGGA | |
| | AGTAAAATCTGAGAAGCTTGACTTCAAGGACAGAGTCCA | |
| | GTCGAAGATTGGGTCCCTGGACAATATCACCCACGTCCC | |
| | TGGCGGAGGAAATAAAAAGATTGAAACCCACAAGCTGAC | |
| | CTTCCGCGAGAACGCCAAAGCCAAGACAGACCACGGGGC | |
| | GGAGATCGTGTACAAGTCGCCAGTGGTGTCTGGGGACAC | |
| | GTCTCCACGGCATCTCAGCAATGTCTCCTCCACCGGCAGC | |
| | ATCGACATGGTAGACTCGCCCCAGCTCGCCACGCTAGCT | |
| | GACGAGGTGTCTGCCTCCCTGGCCAAGCAGGGTTTG | |
| Amino acid | MAEPRQEFEVMEDHAGTYGLGDRKDQGGYTMHQDQEGDTD | 4 |
| sequence for the | AGLKESPLQTPTEDGSEEPGSETSDAKSTPTAEDVTAPLV | |
| 2N4R isoform of | DEGAPGKQAAAQPHTEIPEGTTAEEAGIGDTPSLEDEAAG | |
| human tau with | HVTQARMVSKSKDGTGSDDKKAKGADGKTKIATPRGAAPP | |
| 279, 285, 301, | GQKGQANATRIPAKTPPAPKTPPSSGEPPKSGDRSGYSSP | |
| 303, 317, and 358 | GSPGTPGSRSRTPSLPTPPTREPKKVAVVRTPPKSPSSAK | |
| bolded | SRLQTAPVPMPDLKNVKSKIGSTENLKHQPGGGKVQIINK | |
| | KLDLSNVQSKCGSKDNIKHVPGGGSVQIVYKPVDLSKVTS | |
| | KKCGSLGNIHHPGGGQVEVKSEKLDFKDRVQSKIGSLDNI | |
| | ETHVPGGGNKKITHKLTFRENAKAKTDHGAEIVYKSPVVS | |
| | GDTSPRHLSNVSSTGSIDMVDSPQLATLADEVSASLAKQG | |
| | L | |
| DNA sequence | ATGGCGAACCTTGGCTGCTGGATGCTGGTTCTCTTTGTGG | 5 |
| for WT PRNP | CCACATGGAGTGACCTGGGCCTCTGCAAGAAGCGCCCGA | |
| prion protein | AGCCTGGAGGATGGAACACTGGGGGCAGCCGATACCCGG | |
| | GGCAGGGCAGCCCTGGAGGCAACCGCTACCCACCTCAGG | |
| | GCGGTGGTGGCTGGGGGCAGCCTCATGGTGGTGGCTGGG | |
| | GGCAGCCTCATGGTGGTGGCTGGGGGCAGCCCCATGGTG | |
| | GTGGCTGGGGACAGCCTCATGGTGGTGGCTGGGGTCAAG | |
| | GAGGTGGCACCCACAGTCAGTGGAACAAGCCGAGTAAGC | |
| | CAAAAACCAACATGAAGCACATGGCTGGTGCTGCAGCAG | |
| | CTGGGGCAGTGGTGGGGGGCCTTGGCGGCTACATGCTGG | |
| | GAAGTGCCATGAGCAGGCCCATCATACATTTCGGCAGTG | |
| | ACTATGAGGACCGTTACTATCGTGAAAACATGCACCGTT | |
| | ACCCCAACCAAGTGTACTACAGGCCCATGGATGAGTACA | |
| | GCAACCAGAACAACTTTGTGCACGACTGCGTCAATATCA | |
| | CAATCAAGCAGCACACGGTCACCACAACCACCAAGGGGG | |
| | AGAACTTCACCGAGACCGACGTTAAGATGATGGAGCGCG | |
| | TGGTTGAGCAGATGTGTATCACCCAGTACGAGAGGGAAT | |
| | CTCAGGCCTATTACCAGAGAGGATCGAGCATGGTCCTCT | |
| | TCTCCTCTCCACCTGTGATCCTCCTGATCTCTTTCCTCA | |
| | TCTTCCTGATAGTGGGATGA | |
| Amino acid | MANLGCWMLVLFVATWSDLGLCKKRPKPGGWNTGGSRYP | 6 |
| sequence for | GQGSPGGNRYPPQGGGGWGQPHGGGWGQPHGGGWGQPHG | |
| human PRNP | GGWGQGGGTHSQWNKPSKPKTNMKHMAGAAAAGAVVGGL | |
| prion protein with | GGYMLGSAMSRPIIHFGSDYEDRYYRENMHRYPNQVYYR | |
| amino acids 127, | PMDEYSNQNNFVHDCVNITIKQHTVTTTTKGENFTETDV | |
| 129, and 219 | KMMERVVEQMCITQYERESQAYYQRGSSMVLFSSPPVIL | |
| bolded | LISFLIFLIVG | |

### DESCRIPTION OF THE EMBODIMENTS

Item 21. A method to diagnose a neurodegenerative disease comprising: In protein misfolding diseases, a normal, globular protein misfolds into a flat conformation (or an amyloid) that is capable of self-templating. This is often referred to as the prion mechanism of disease. Provided herein is a cell-based assay that demonstrates that one can insert a single point mutation into a protein, for example, tau and α-synuclein, which cause Alzheimer's and Parkinson's diseases, respectively, to prevent propagation of a misfolded conformation. For example, one can use the E46K, K80E, and K80W mutations to block propagation of α-synuclein aggregates isolated from multiple system atrophy (MSA) patient samples in cells.

Provided herein are techniques to insert one of these protective mutations as a novel approach to treating disease. In some embodiments, gene therapy uses a gene therapy vector, such as adeno-associated viruses (AAVs), to deliver an exogenous copy of a mutant gene to a patient's cells. In this way, the gene therapy leads to expression of a mutant protein, wherein this mutant protein blocks misfolding of an endogenously expressed protein. In this way, expression of the mutant protein can interferes with disease progression caused by misfolded endogenously expressed protein.

Alternatively, one can use a vector, such as AAVs, to deliver the CRISPR prime editing machinery to edit the gene encoding the endogenously expressed protein, thus inhibiting misfolding of the endogenously expressed protein and preventing the disease from progressing. As used herein, CRISPR prime editing refers to gene editing for introducing a gene mutation encoding for a mutant protein capable of blocking an endogenously expressed protein in the subject from adopting a misfolded conformation. Throughout the application, gene editing, CRISPR, or CRISPR prime refer to and include any method of gene editing. In some embodiments, the patient's endogenously expressed protein with a misfolded conformation is responsible for a disorder, as described herein.

An aspect of the present gene editing and gene therapy approaches that makes them superior to other gene therapy approaches to protein misfolding diseases is that they will maintain the normal function of the protein and only disrupt the disease-causing events (expression of the disease-causing protein is not blocked and, if present, the disease-causing mutation is not necessarily corrected, but rather a mutant protein is expressed so the endogenously expressed protein cannot fold into the disease-causing conformation).

Also described herein will be means of exogenous delivery of a mutant protein to decrease misfolding of a protein endogenously expressed by the patient.

While the data for this invention are related to the proteins tau, prion protein, and alpha-synuclein, the approach set forth herein has a broader use. This same strategy can be used to interfere with protein misfolding for any diseases that rely on the prion mechanism. Moreover, while gene therapy has, so far, been used to correct a disease-causing mutation, the approach provided herein can be used to treat patients with both familial and sporadic disease.

In addition to gene therapy uses, the present technology can also be used for diagnosing neurodegenerative disease. For example, the N279K mutation in tau allows propagation of tau aggregates isolated from corticobasal degeneration patients, but not from progressive supranuclear palsy patients. These mutations, which were selected based on their ability to interfere with protein misfolding, can therefore serve as a tool for diagnosing disease in a patient, and then matching the patient with the needed gene therapy.

Herein provided is: 1) the use of diagnostics that allow rapid identification of the neurodegenerative disease or protein misfolding disease of a patient. At the present time, many neurodegenerative diseases are diagnosed only by autopsy, and incorrect diagnoses during early stages of disease are common; 2) gene therapeutics (gene therapy or gene editing) that slow or stop the progression of disease; and 3) exogenous delivery of a mutant protein to decrease misfolding of a protein endogenously expressed by the patient. Currently there are no therapeutics that alter the course of most of these diseases.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, several embodiments with regards to methods and materials are described herein. As used herein, each of the following terms has the meaning associated with it in this section.

For the purposes of clarity and a concise description, features can be described herein as part of the same or separate embodiments: however, it will be appreciated that the scope of the invention may include embodiments having combinations of all or some of the features described.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

As used herein, the indefinite articles “a”, “an” and “the” should be understood to include plural reference unless the context clearly indicates otherwise.

The phrase “and/or,” as used herein, should be understood to mean “either or both” of the elements so conjoined, e.g., elements that are conjunctively present in some cases and disjunctively present in other cases.

As used herein, “or” should be understood to have the same meaning as “and/or” as defined above. For example, when separating a listing of items, “and/or” or “or” shall be interpreted as being inclusive, e.g., the inclusion of at least one, but also including more than one, of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the terms “including”, “includes”, “having”, “has”, “with”, or variants thereof, are intended to be inclusive similar to the term “comprising.”

The terms “comprises,” “comprising,” and the like can have the meaning ascribed to them in US Patent Law and can mean “includes,” “including” and the like. As used herein, “including” or “includes” or the like means including, without limitation.

As used herein, the term “about” means plus or minus 10% of the indicated value. For example, about 100 means from 90 to 110. Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about.”

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

The term “standard,” as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

As used herein, an “effective amount” means an amount sufficient to produce a selected effect, such as alleviating symptoms of a disease or disorder. In the context of administering two or more compounds, the amount of each compound, when administered in combination with another compound(s), may be different from when that compound is administered alone. The term “more effective” means that the selected effect is alleviated to a greater extent by one treatment relative to the second treatment to which it is being compared.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

The term “prevent,” as used herein, means to stop something from happening, or taking advance measures against something possible or probable from happening. In the context of medicine, “prevention” generally refers to action taken to decrease the chance of getting a disease or condition.

A “sample,” as used herein, refers to a biological sample from a subject, including, but not limited to, normal tissue samples, diseased tissue samples (e.g., brain), biopsies, blood, saliva, feces, semen, tears, and urine. A sample can also be any other source of material obtained from a subject which contains cells, tissues, or fluid of interest as interpreted in the context of the claim and the type of assay to be performed using that sample.

A “subject” of diagnosis or treatment is a mammal, including a human.

As used herein, the term “treating” may include prophylaxis of the specific disease, disorder, or condition, or alleviation of the symptoms associated with a specific disease, disorder, or condition and/or preventing or eliminating the symptoms. A “prophylactic” treatment is a treatment administered to a subject who does not exhibit signs of a disease or exhibits only early signs of the disease for the purpose of decreasing the risk of developing pathology associated with the disease. “Treating” is used interchangeably with “treatment” herein.

Methods involving conventional molecular biology techniques are described herein. Such techniques are generally known in the art and are described in detail in methodology treatises, such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Methods for chemical synthesis of nucleic acids are discussed, for example, in Beaucage and Carruthers, Tetra. Letts. 22:1859-1862, 1981, and Matteucci et al., J. Am. Chem. Soc. 103:3185, 1981.

The term “prion” or “prion mechanism” is broadly used to describe self-templated protein misfolding or a misfolded protein that can act as a template for additional protein misfolding. During this process, a protein that typically exists in a globular, 3-dimensional structure undergoes a conformational change into an abnormal conformation that is usually planar. The resulting flat conformation can then serve as a catalytic surface resulting in the induced misfolding of the same type of protein into the same shape (for example, misfolded prion protein can induce the misfolding of another copy of the prion protein). When this occurs, hydrogen bonds form between the carbon backbones of the proteins, substantially stabilizing the misfolded proteins as more proteins misfold forming a fibril. This mechanism was first introduced in 1982 to explain how the prion protein causes progressive neurodegeneration in animals that develop spongiform encephalopathies (e.g., Creutzfeldt-Jakob disease in humans, bovine spongiform encephalopathy in cattle, scrapie in sheep and goats, and chronic wasting disease in deer, elk, mouse, and reindeer).

Several additional proteins, including tau, alpha-synuclein, and beta-amyloid, use this same mechanism to cause disease. While some researchers refer to these proteins as prions, others use the term prion-like to differentiate between PrP and non-PrP proteins; however, throughout this application all of these will be considered within the definition of “prion” or “prion mechanism”. On occasion, in the art other terms such as prionoid or self-templating proteins are used instead of prion or prion-like and this application will treat those terms as synonyms, as well. Ultimately, these terms all refer to the same underlying disease mechanism, even if the field is currently grappling with which terminology should be used.

Diseases and Genes Associated with Prions

Specific diseases that are identifiable in the diagnostic assay and/or are treatable include, but are not limited to, tau-related diseases (or tauopathies), including, but not limited to, Alzheimer's disease (in which the proteins Aβ, ApoE, and TMEM106B also contribute to the disease and may be targeted using the same approach), progressive supranuclear palsy, chronic traumatic encephalopathy, Pick's disease, argyrophilic grain disease, and corticobasal degeneration: α-synuclein-related diseases (or synucleinopathies), including, but not limited to, Parkinson's disease, multiple system atrophy (MSA), Lewy body diseases, and Parkinson's disease with dementia: Huntington's disease (caused by mutant Htt), amyotrophic lateral sclerosis (ALS: caused by TDP-43 and/or SOD1), diabetes, prion protein (PrP)-related diseases, including but not limited to, Creutzfeldt-Jakob disease, fatal familial insomnia, and Gerstmann-Sträussler-Scheinker disease.

Non-disease-causing amino acid substitutions that do not interfere with normal protein function can be used in the invention described herein.

Strategies for selecting strain-specific mutations can be based on three-dimensional models of the disease-causing proteins. Specific amino acid mutations can be selected using one or more or the following criteria: for example, (1) impeding an existing salt bridge, such as replacing a lysine with a neutral amino acid such as phenylalanine or an acidic amino acid like aspartic acid or glutamic acid which creates Coulombic repulsion. The acidic half of the salt bridge can also be substituted with either a neutral amino acid or a lysine or asparagine. Replacing aspartic acid with glutamic acid or vice versa can also disrupt the salt bridge: for example, (2) replacing a neutral residue with a charged residue can disfavor protein conformations that place the charge in a hydrophobic region. This creates an unfavorable charge imbalance if counterions cannot access the residue to neutralize the charge. In another example, (3) changing the size of a residue can render a protein incompatible with a specific conformation. A large residue could create a steric clash incompatible with particular folding patterns. A small residue may leave an empty space or vacuum inside the aggregate that would be energetically unfavorable. Changing the amino acid size can also prevent zipper formation in the misfolded aggregate protein: for example, (4) preventing the protein from making a turn or loop. Loops and turns in misfolded proteins have specific conformational requirements. Adding a conformationally restricted amino acid, such as replacing glycine with valine, will stop misfolding of particular conformations by preventing the turn.

Amino acids to be mutated can be selected on the basis of factors including impeding salt bridge formation: maintaining charge but using a large amino acid: changing charge or creating a charge (such changes could create an energetically unfavorable charge imbalance): introducing an amino acid mutation that prevents the protein from making a turn required for misfolding (such as changing a glycine); and/or changing size to disfavor zipper formation. For example, one could mutate a given residue for a larger one such as mutating an aspartic acid to a larger, polar residue (like tyrosine or glutamic acid). Notably, these mutations are carefully selected to interfere with the misfolded protein conformation while avoiding disrupting normal protein function.

As described herein, a disease associated with a prion can be treated with gene therapy to express a mutant protective protein, gene editing (such as CRISPR prime editing as described below) to express a mutant protective protein, or exogenous administration of a mutant protective protein. In some embodiments, the mutant protective protein is one that can inhibit misfolding of an endogenously expressed protein (such as a prion). In some embodiments, the efficacy of the treatment does not require that expression of the endogenously expressed protein be altered, but instead that exogenous mutant protein can block misfolding of the endogenously expressed protein. In some embodiments, the mutant protein can treat disease without needing to be expressed at levels similar or equal to that of the endogenously expressed protein. In other words, a relatively low level of mutant protein may be able to block misfolding of the endogenously expressed protein.

Described herein are mutant nucleic acids encoding for mutant proteins, mutant proteins, and cell lines comprising a nucleic acid that encodes for a mutant protein.

### MSA/α-syn

For example, MSA is an invariably fatal movement disorder that lacks effective treatment. While some groups have proposed using gene therapy strategies to reduce α-syn expression in neurons, thus eliminating the substrate needed for disease propagation, there are concerns that this approach could lead to loss-of-function deficits in patients. As an alternative, an approach disclosed herein is gene therapy or gene editing via CRISPR prime editing (or other gene editing techniques) to generate conversion-incompetent α-syn, thus blocking the ability of the protein to adopt the misfolded conformation responsible for MSA. This concept represents a paradigm-shift in how one approaches gene therapy and offers an innovative strategy for clinical intervention. For example, the present method may allow for the mutant protein to be expressed at relatively low levels, but still be capable of blocking the endogenously expressed protein in the subject from adopting a misfolded conformation.

Descriptions and variants of α-synuclein are described at Gene ID: 6622 and UniProt ID: P37840, as well as SEQ ID NOs: 1 and 2.

Mutations in the templating region of α-synuclein, which spans amino acids 14-99, have been associated with Parkinson's disease, including A30G/P, H50Q, G51D, E46K, A53E/T/V, and T72M. In some embodiments, mutant proteins as described herein may be targeted to inhibit misfolding of α-synuclein due to one or more of these clinically relevant mutations. In some embodiments, α-synuclein regions to mutate include the templating regions (amino acids 14-99). For example, in some embodiments one or more of the following mutations may be employed: A30G, A53E, E46K, A53E, T72M, and K80E/W.

In some embodiments, mutant proteins described herein may comprise. substitutions at K58, E35, E28, K60, K23, D98, K9, E20, K21, K58, D57, Q79, S87, T81, Q24, T64, N65, T75, T92, T81, G25, A27, A29, G31, T33, L38, V40, S42, T44, E83, G36, G41, G47, G51, G67, G73, G84, G86, G93, T22, T72, H50, Y39, K45, E46, K47, D61, T54, and/or T59, as described below, in order to block misfolding of α-synuclein.

As described herein, the E46K mutation in α-synuclein is used to establish proof-of-concept for this approach. However, knowing that the E46K mutation causes familial Parkinson's disease (Zarranz J J et al. Ann Neurol 2004: 55 (2):164-73), other non-disease-causing mutations that also prevent MSA propagation in vitro have been identified. For example, to interfere with the salt bridge between E46 and K80 in α-synuclein (Schweighauser M et al. Nature 2020: 585 (7825):464-9) the effect of mutating K80 to an uncharged residue to prevent salt bridge formation was tested, for example, with E46K, K80E, and K80W mutations. SEQ ID NO:1 and SEQ ID NO:2 represent the DNA and amino acid sequences of WT α-synuclein, respectively.

In some embodiments, the nucleic acid comprises two or more, three or more, four or more, or five or more amino acid substitutions as compared to SEQ ID NO: 2. In some embodiments, the two or more amino acid substitutions comprise E46K and K80E.

Further a mutation of α-synuclein can include;
mutation that disrupts a misfolded conformation of α-synuclein, which in some embodiments excludes known mutations that correlate to human neurodegenerative disease;
mutation that disrupts a misfolded conformation of α-synuclein, which in some embodiments excludes: A30G, A30P, E46K, H50Q, G51D, A53E, A53V, T72M, E83Q, and A53T;
mutation in the misfolded region of α-synuclein which constitutes residues 14-99 (based on CryoEM structures);
mutation that disrupts a salt bridge in a misfolded structure of α-synuclein;
mutation that disrupts a salt bridge in a misfolded structure of α-synuclein such as K58, E35, E28, K60, E46, K80, K23, D98, K9, E20, K21, K58, and/or D57;
mutation that disrupts a salt bridge by changing a positively charged lysine residue to a negatively charged glutamic acid or aspartic acid;
mutation that disrupts a salt bridge by changing a negatively charged glutamic acid or aspartic acid residue to a positively charged lysine, arginine, or histidine.
mutation that disrupts a salt bridge by changing a charged residue to a neutral residue (alanine, cysteine, phenylalanine, glycine, isoleucine, lysine, leucine, methionine, asparagine, proline, glutamine, serine, threonine, valine, tryptophan, or tyrosine;
mutation that disrupts a salt bridge by changing the distance between the charged atoms by replacing one negatively charged amino acid (glutamic acid or aspartic acid) for the other;
mutation that disrupts a hydrogen bond, a dipole-dipole interaction, or a charge, dipole interaction between two amino acids;
mutation of Q79, S87, T81, Q24, T64, N65, T75, T92, and/or T81 to an amino acid that lacks a hydrogen bond donor;
mutation of Q79, S87, T81, Q24, T64, N65, T75, T92, and/or T81 to alanine, valine, isoleucine, leucine, methionine, glycine, proline, or phenylalanine;
mutation that disrupts a van der Waals or Zipper interaction between two amino acids;
mutation that creates a steric clash with another amino acid in the misfolded conformation;
mutation that replaces a large amino acid with a smaller one, creating a hole that cannot be filled by other atoms or solvent;
mutation in the zippered sequence between 25 and 44, mutation of G25, A27, A29, G31, T33, L38, V40, S42 and/or T44 to a different amino acid;
mutation in the zippered sequence between 25 and 44, mutation of G25, A27, A29, G31, T33, L38, V40, S42 and/or T44 to a larger amino acid;
mutation in the zippered sequence between 25 and 44, mutation of G25, A27, A29, G31, T33, L38, V40, S42 and/or T44 to a charged amino acid;
mutation in the zippered sequence between 25 and 44, mutation of A27, A29, T33, L38, V40, S42 and/or T44 to a smaller amino acid;
mutation that changes the phi and psi angles of an amino acid such that the angles required to maintain the misfolded conformation become disallowed;
mutation of amino acids with s positive phi angle to amino acids which rarely adopt positive phi angles due to conformational strain;
mutation of E83, K58, and/or K21 to amino acids that rarely adopt positive phi angles;
mutation of E83, K58, and/or K21 to amino acids to proline, valine, isoleucine, methionine, threonine, or tryptophan;
mutation of glycines to any other amino acid;
mutation of glycines with phi or psi angles that are disallowed for other amino acids;
mutation of glycines with positive phi angles to amino acids that rarely adopt positive phi angles;
mutation of glycines with positive phi angles to proline, valine, isoleucine, methionine, threonine, or tryptophan;
mutation of G25, G36, G41, G47, G51, G67, G73, G84, G86, and/or G93 to any other amino acid;
mutation of G25, G36, G41, G47, G51, G67, G73, G84, G86, and/or G93 to proline, valine, isoleucine, methionine, threonine, or tryptophan;
mutation of amino acids with psi angles less than-60 degrees to another amino acid;
mutation of amino acids with psi angles less than-60 degrees to an amino acid that rarely adopts a psi angle less than-60 degrees;
mutation of amino acids with psi angles less than-60 degrees to cysteine, alanine, glutamic acid, glutamine, isoleucine, leucine, lysine, histidine, tryptophan, valine, or tyrosine;
mutation of T22, G31, G36, G68, T72, G73, E83, G84, and/or D98 to an amino acid that rarely adopts a psi angle less than-60 degrees;
mutation of T22, G31, G36, G68, T72, G73, E83, G84, and/or D98 to cysteine, alanine, glutamic acid, glutamine, isoleucine, leucine, lysine, histidine, tryptophan, valine, or tyrosine;
mutation that disrupts a network of polar or chelating groups in the misfolded conformation;
mutation that disrupts the network of H50, Y39, K45 and K47;
mutation of H50, K45 and/or K47 to hydrophobic, nonchelating amino acid such as alanine, cysteine, phenylalanine, glycine, isoleucine, lysine, leucine, methionine, asparagine, proline, glutamine, serine, threonine, valine, tryptophan, or tyrosine;
mutation of H50, K45 and/or K47 to negatively charged aspartic acid or glutamic acid to prevent chelation to negatively charged species;
mutation of Y39 to another amino acid;
mutation that disrupts the network of D61, T59, T54 and T72;
mutation of D61 to a nonpolar amino acid or a basic amino acid;
mutation of T59, T54 and/or T72 to a nonpolar amino acid such as alanine, cysteine; phenylalanine, glycine, isoleucine, lysine, leucine, methionine, asparagine, proline, glutamine, valine, tryptophan, or tyrosine, an acidic amino acid, or a basic amino acid;
the A30G mutation;
the A53E mutation;
the E46K mutation;
the V55Y mutation;
the A53E mutation;
the T72M mutation;
the V74I mutation; and/or
the K80E, K80N and K80W mutation.

### Tauopathies

Tauopathies are invariably fatal neurodegenerative disorders that are characterized either as memory or movement disorders. In some embodiments, tauopathies are related to aggregation of tau. All tauopathies lack any effective treatment. While some groups have proposed using gene therapy strategies to reduce expression of particular tau isoforms in neurons, eliminating the substrate needed for disease propagation, there are concerns that this approach could lead to loss-of-function deficits in patients. As an alternative, proposed herein is a novel application of gene therapy or gene editing (i.e., CRISPR prime editing or other gene editing) to generate conversion-incompetent tau, thus blocking the ability of the protein to adopt the misfolded conformation responsible for disorders including Alzheimer's disease (AD), progressive supranuclear palsy (PSP), and corticobasal degeneration (CBD). This concept represents a paradigm-shift in how one approaches gene therapy and offers an innovative strategy for clinical intervention.

Descriptions and variants of tau are described at Gene ID: 4137 and UniProt ID: P10636, as well as SEQ ID NOs: 3 and 4.

Several tau mutations including N279K, S285R, P301S/L, and V337M have been associated with neurological disorders. Other known tau mutations include those in templating regions, which span amino acids 254-378 in 3R tau for Pick's disease, amino acids 306-378 in 3R and 4R tau for Alzheimer's disease and chronic traumatic encephalopathy, and amino acids 244-380 in 4R tau for corticobasal degeneration and progressive supranuclear palsy, or other 4R tauopathies. In some embodiments, mutant proteins as described herein may be targeted to inhibit misfolding of tau due to one or more of these clinically relevant mutations. In some embodiments, tau regions to mutate include templating regions spanning amino acids 254-378, amino acids 306-378, or amino acids 244-380, depending upon disease. For example, in some embodiments one or more of the following mutations may be employed: G303V, N279K, S285R, K317H, G323F, G333A/I, G334F/W/A/I, G335A/I, D358E, G366A/I, and/or G367M/V.

Herein, the N279K and S285R mutations were used to establish proof-of-concept for this approach. However, knowing that the N279K and S285R mutations may be factors in development of PSP, other non-disease-causing mutations that also prevent PSP propagation in vitro will be identified.

One can also use mutated tau, for example G303V, K317H, D358E, N279K, G367M/V, G323F, G334F/W/A/I, G335A/I, G333A/I, G366A/I and/or S285R so as to inhibit protein misfolding and aggregate formation and prevent disease propagation. The position of these residues is highlighted in the DNA (SEQ ID NO: 3) and amino acid sequences (SEQ ID NO: 4) of the 2N4R isoform of human tau. In some embodiments, the nucleic acid comprises two or more, three or more, four or more, or five or more amino acid substitutions as compared to SEQ ID NO: 4.

Further mutations of interest in tau include;
mutation that disrupts a misfolded conformation of tau, which in some embodiments excludes known mutations that correlate to human neurodegenerative disease;
mutation that disrupts a misfolded conformation of tau, which in some embodiments excludes: P4T, R5C, R5H, R5L, H14H, T17M, Y18Y, T30A, T39T, A41T, G55R, V75A, G86S, A90V, A152T, P176P, A178T, G200E, P200P, P202L, R211H, G213R, V224G, A227A, Q230R, A239T, D252V, N255N, K257T, I260V, L266V, L266L, P270P, G272V, G273R, N279K, K280del, L284R, L284L, D285N, D285D, S285R, V287I, V289A, C291R, N296H, N296D, N296N, N296del (AN296), A297V, V300I, P301T, P301S, P301L, P301P, G303V, S305N, S305I, S305S, L315R, L315L, K317M, K317N, S318L, S320F, S320Y, P332S, G335S, G335A, G335V, Q336R, Q336H, V337M (Seattle), E342V, S352L, S356T, I360V, V363I, P364S, G366R, K369I, R370W, E372G, G389_I392del, G389R (G>A), G389R (G>C), P397S, R406W, L410F, N410H, S427F, T427M, H441Y, S447P, R448*, T504T, P511R, and P512H.
mutation in the misfolded region of tau which constitutes residues 255-426 (based on CryoEM structures), including G303V;
mutation that disrupts a salt bridge in a misfolded structure of tau;
mutation that disrupts a salt bridge by changing a positively charged lysine residue to a negatively charged glutamic acid or aspartic acid;
mutation that disrupts a salt bridge in a misfolded structure of tau where the positively charged component is K259, K267, K280, K281, K290, K294, K298, K311, K317, K321, K331, K340, K343, K347, K353, K369, K370, K375, K395, K438 H299, H329, H330, H362, H374, H407, and/or R349 (based on CryoEM structures);
mutation that disrupts a salt bridge by changing a negatively charged glutamic acid or aspartic acid residue to a positively charged lysine, arginine, or histidine;
mutation that disrupts a salt bridge in a misfolded structure of tau where the negatively charged component is D283, D285, D314, D348, E358, D421, É64, E338, E342, E372, and/or E380;
mutation that disrupts a salt bridge by changing a charged residue to a neutral residue (alanine, cysteine, phenylalanine, glycine, isoleucine, lysine, leucine, methionine, asparagine, proline, glutamine, serine, threonine, valine, tryptophan, or tyrosine;
mutation that disrupts a salt bridge by changing the distance between the charged atoms by replacing one negatively charged amino acid (glutamic acid or aspartic acid) for the other;
mutation that disrupts a hydrogen bond, a dipole-dipole interaction, or a charge-dipole interaction between two amino acids;
mutation of a polar amino acid such as asparagine, glutamine, serine, threonine, or tyrosine to an amino acid that lacks a hydrogen bond donor;
mutation of asparagine, glutamine, serine, threonine and tyrosine to alanine, valine, isoleucine, leucine, methionine, glycine, proline, or phenylalanine;
mutation that disrupts a van der Waals or Zipper interaction between two amino acids;
mutation that creates a steric clash with another amino acid in the misfolded conformation;
mutation that replaces a large amino acid with a smaller one, creating a hole that cannot be filled by other atoms or solvent;
mutation that changes the phi and psi angles of an amino acid such that the angles required to maintain the misfolded conformation become disallowed;
mutation of an amino acid known to adopt an outlier conformation in a misfolded structured as defined by its typical phi and psi angles to an amino acid calculated to require a higher energy barrier for the required conformation;
mutation of an amino acid known to adopt an outlier conformation in a misfolded structure as defined by its typical phi and psi angles to an amino acid calculated to require a higher energy barrier for the required conformation including rigid amino acids such as proline, valine, isoleucine, methionine, threonine, or tryptophan;
mutation of an amino acid such as G272, K274, V275, I277, K298, G304, S305, V306, V309, K317, N327, I328, D348, P364, N368, T377, F378, R379, E380, H388, and/or E391 known to adopt an outlier conformation in a misfolded tau structures defined by its typical phi and psi angles to a different amino acid calculated to require a higher energy barrier for the required conformation including rigid amino acids such as proline, valine, isoleucine, methionine, threonine or tryptophan;
mutation of amino acids in tau with a positive phi angle to amino acids which rarely adopt positive phi angles due to conformational strain;
mutation of Q269, K274, V275, I277, K280, K281, L284, N286, Q288, K290, S293, K294, D295, N296, K298, H299, S305, V306, Q307, 1308, V309, K311, L315, K317, C322, S324, L325, N327, I328, H329, K331, E338, K343, D345, D348, R349, L357, D358, N359, I360, V363, N368, K370, H374, L376, R379, E380, N381, H388, E391, K395, H407, D421, A426, D430, E431, and/or A434 to amino acids that rarely adopt positive phi angles;
mutation of Q269, K274, V275, I277, K280, K281, L284, N286, Q288, K290, S293, K294, D295, N296, K298, H299, S305, V306, Q307, 1308, V309, K311, L315, K317, C322, S324, L325, N327, I328, H329, K331, E338, K343, D345, D348, R349, L357, D358, N359, I360, V363, N368, K370, H374, L376, R379, E380, N381, H388, E391, K395, H407, D421, A426, D430, E431, and/or A434 to amino acids to proline, valine, isoleucine, methionine, threonine or tryptophan;
mutation of glycine to any other amino acid;
mutation of glycine with phi or psi angles that are disallowed for other amino acids;
mutation of glycine with positive phi angles to amino acids that rarely adopt positive phi angles;
mutation of glycine with positive phi angles to proline, valine, isoleucine, methionine, threonine, or tryptophan;
mutation of glycine known to have a positive phi angle in one or more tau structures such as G261, G271, G272, G273, G292, G302, G303, G304, G323, G326, G333, G334, G335, G355, G365, G366, G367, G389, G401, G415, and/or G440 to any other amino acid;
mutation of G261, G271, G272, G273, G292, G302, G303, G304, G323, G326, G333, G334, G335, G355, G365, G366, G367, G389, G401, G415, and/or G440 to proline, valine, isoleucine, methionine, threonine, or tryptophan;
mutation of amino acid with psi angles less than-60 degrees to another amino acid;
mutation of amino acid with psi angles less than-60 degrees to an amino acid that rarely adopts a psi angle less than-60 degrees;
mutation of amino acid with psi angles less than-60 degrees to cysteine, alanine, glutamic acid, glutamine, isoleucine, leucine, lysine, histidine, tryptophan, valine, or tyrosine;
mutation of amino acids with psi angles less than-60 degrees in a misfolded conformation such as G271, G272, G273, K274, I277, K281, D283, S285, N286, Q288, S289, G292, N296, K298, G302, G303, G304, S305, Y310, K317, S320, C322, G323, S324, L325, G326, I328, H330, P332, G333, G334, G335, E338, S341, E342, K347, D348, S352, I354, G355, S356, D358, H362, P364, G365, G366, G367, T377, F378, R379, E380, N381, H388, G401, A426, and/or G440 to an amino acid that rarely adopts a psi angle less than-60 degrees;
mutation of G271, G272, G273, K274, I277, K281, D283, S285, N286, Q288, S289, G292, N296, K298, G302, G303, G304, S305, Y310, K317, S320, C322, G323, S324, L325, G326, I328, H330, P332, G333, G334, G335, E338, S341, E342, K347, D348, S352, I354, G355, S356, D358, H362, P364, G365, G366, G367, T377, F378, R379, E380, N381, H388, G401, A426, and/or G440 to cysteine, alanine, glutamic acid, glutamine, isoleucine, leucine, lysine, histidine, tryptophan, valine, or tyrosine;
mutation that disrupts a network of polar or chelating groups in the misfolded conformation;
mutation that disrupts one of the tau polar networks;
mutation that disrupts one of the following polar networks:
   K317, T319, K321, N327, H329, K331;
   K347, D345, K343, E342, H362, D358, S356, K353, Q351;
   K340, E338, Q336, K331, E380, T377, D375, T373, K369;
   K395, M419, S422, Q424, Y394;
   K317, T319, K321, S324, N327, H329, E338, K340, E342;
   K317, T319, K321, E338, K340, E342, K343;
   N327, H329, K331;
   R349, Q351, K353, D358, H362;
   R349, Q351, K353, D358, H362, K369, T373, K375, T377, R379;
   N327, H329, K331, E338, K340, E342, K343, D345, K347;
   K317, T319, K321, N327, H329, K331, Q336;
   R349, Q351, K353, D358, H362, R349, Q351, K353, D358, H362;
   K317, T319, K321;
   H329, K331, S324;
   H330, K331, K317, T319, K321;
   H330, K331, D348, R349;
   N279, K281, D283, S285, K290, S293, D295, K298, H299;
   S324, N327, H329, K331;
   K311, K317, T319, K321, N327, H329, K331;
   K281, D283, K290, S293, D295, K298, K299;
   R349, Q351, K353, D358, H362, K369, T373, K375, T377, R379; or
   K340, E342, K343, D345, K347, wherein, in some embodiments, a single mutation may be capable of disrupting the polar network (such as by interfering with ionic salts that have rigorous shape and geometry requirements);
mutation of a polar network amino acid to a hydrophobic, nonchelating amino acid such as alanine, cysteine, phenylalanine, glycine, isoleucine, lysine, leucine, methionine, asparagine, proline, glutamine, serine, threonine, valine, tryptophan, or tyrosine;
mutation of a polar network amino acid to a negatively charged aspartic acid or glutamic acid to prevent chelation to negatively charged species;
mutation of a polar network amino acid to a nonpolar amino acid or a basic amino acid;
mutation of a polar network amino acid to a nonpolar amino acid such as alanine, cysteine, phenylalanine, glycine, isoleucine, lysine, leucine, methionine, asparagine, proline, glutamine, valine, tryptophan, or tyrosine, an acidic amino acid, or a basic amino acid; and/or mutation of polar network amino acids.

### Prion Protein

Prion protein (PrP, PRNP) is associated with a number of diseases characterized by rapidly progressing neurodegeneration. There are known protective polymorphisms for PrP that developed naturally (see, for example, Kai et al., Heliyon 9:e13974 (2023)), including E219K. At residue 219 of PrP, humans are most protected from developing prion disease when they are heterozygous for the E/K polymorphism (1 copy of E and 1 copy of K).

Descriptions and variants of PrP are described at Gene ID: 5621 and UniProt ID: P04156, as well as SEQ ID NOs: 5 and 6.

Some known PrP mutations include those within the region of amino acids 127-219.

Mutations known to be protective against developing disease associated with misfolded PrP include E219K. Further, 219E/K polymorphism, 129M/V polymorphism, and 127V/G polymorphism are also known to be protective.

In some embodiments, a method to treat a neurodegenerative disease comprises introducing a mutation in vivo in the gene of the protein that is misfolding in a subject in need thereof, wherein the neurodegenerative disease is the result of protein misfolding and aggregate formation, wherein the mutation renders the subject functionally heterozygous. As used herein, “functionally heterozygous” refers to a subject who naturally expresses 2 copies of the same allele and therefore is homozygous in expression of a given protein, wherein this subject is treated to introduce a mutant nucleic acid that encodes for a different variant of the same protein (i.e., the two proteins are single nucleotide polymorphisms of each other or other variants). In this case, the subject would then express two different variants of the same protein, such as prion protein.

As used herein, exogenous delivery can be via administration of a protein of interest, without requiring gene editing or gene therapy. For example, a prion protein with a specific amino acid substitution can be prepared in vitro and supplied to a patient.

In some embodiments, a subject in need of treatment for a prion disease associated with PrP is genotyped to determine what sequence they carry on both alleles encoding for PrP. In some embodiments, if a patient's genotype encodes for EE or KK at residue 219 of PrP, gene editing, gene therapy, or exogenous delivery, as described herein, can be used to introduce a mutation encoding for K or E, respectively, at residue 219 or to supply a prion protein comprising K or E at residue 219, respectively, and render the patient functionally heterozygous for residue 219 of PrP.

In some embodiments, gene editing, gene therapy, or exogenous delivery is used to introduce a mutation encoding E219K to a subject whose genotype encodes EE at residue 219 of PrP for treatment of a disease associated with PrP. In some embodiments, gene editing, gene therapy, or exogenous delivery is used to introduce a mutation encoding K219E or to supply a prion protein comprising E219 to a subject whose genotype encodes KK at residue 219 of PrP for treatment of a disease associated with PrP.

Patients who are heterozygous for M/V polymorphism at residue 129 of PrP (1 copy of M and 1 copy of V) are most protected from developing prion disease. In some embodiments, a subject in need of treatment for a prion disease associated with PrP is genotyped to determine what sequence they carry on both alleles of PrP. In some embodiments, if a patient's genotype encodes for MM or VV at residue 129 of PrP, gene editing, gene therapy, or exogenous delivery, as described herein, can be used to introduce a mutation encoding for the M or V sequence and render the patient functionally heterozygous for residue 129 of PrP. Alternatively, a prion protein comprising M129 (if the patient endogenously expresses V129) or V129 (if the patient endogenously expresses M129) can be administered to the patient.

In some embodiments, gene editing, gene therapy, or exogenous delivery is used to introduce a mutation encoding V127M or to supply a prion protein comprising M127 to a subject whose genotype encodes VV at residue 129 of PrP for treatment of a disease associated with PrP. In some embodiments, gene editing, gene therapy, or exogenous delivery is used to introduce a mutation encoding M219V or to supply a prion protein comprising V219 to a subject whose genotype encodes MM at residue 129 of PrP for treatment of a disease associated with PrP.

Patients who are heterozygous for G/V polymorphism at residue 127 of PrP (1 copy of G and 1 copy of V) are most protected from developing prion disease. In some embodiments, a subject in need of treatment for a prion disease associated with PrP is genotyped to determine what sequence they carry on both alleles of PrP. In some embodiments, if a patient's genotype encodes GG or VV at residue 127 of PrP, gene editing, gene therapy, or exogenous delivery, as described herein, can be used to introduce a mutation encoding the G or V sequence or to supply a prion protein comprising G127 or V127 and render the patient functionally heterozygous for residue 127 of PrP.

In some embodiments, gene editing, gene therapy, or exogenous delivery is used to introduce a mutation encoding V127G or to supply a prion protein comprising G127 to a subject whose genotype encodes VV at residue 127 of PrP for treatment of a disease associated with PrP. In some embodiments, gene editing, gene therapy, or exogenous delivery is used to introduce a mutation encoding G217V or to supply a prion protein comprising V217 to a subject whose genotype encodes GG at residue 127 of PrP for treatment of a disease associated with PrP.

In some embodiments:
the subject expresses E219, and the mutation is E219K;
the subject expresses K219, and the mutation is K219E;
the subject expresses M129, and the mutation is M129V;
the subject expresses V129, and the mutation is V129M;
the subject expresses V127, and the mutation is V127G; or
the subject expresses G127, and the mutation is G127V.

In some embodiments, disease symptoms become less severe as the subject expresses more mutant prion protein from a gene introduced via gene therapy or gene editing. In some embodiments, disease symptoms become less severe as the subject is administered a higher dose of a mutant prion protein. While representative examples of residues that may be used are described herein, these examples do not limit the present methods.

### Disease/Gene Mutation Examples

Described herein are further examples of diseases and/or gene mutations that may be treated by the present methods. However, the present methods are broadly applicable to different proteins and other mutations of the listed proteins. Strategies for selecting strain-specific mutations, such as impeding an existing salt bridge, may be used as described above.

β-amyloid (cleaved from APP protein) is described at Gene ID: 351 and UniProt ID: P05067. Known β-amyloid mutations include those in templating region, which spans amino acids 9-42. In some embodiments, β-amyloid regions to mutate include the templating region spanning amino acids 9-42.

TDP-43 is described at Gene ID: 23435 and UniProt ID: Q13148.

SOD1 is described at Gene ID: 6647 and UniProt ID: P00441.

HTT is described at Gene ID: 3064 and UniProt ID: P42858.

ApoE is described at Gene ID: 348 and UniProt ID: P02649.

TMEM106B is described at Gene ID: 54664 and UniProt ID: Q9NUM4. Known TMEM106B mutations include those in templating region, which spans amino acids 120-254. In some embodiments, TMEM106B regions to mutate include the templating region spanning amino acids 120-254.

### Gene Editing and Gene Therapy Strategies/Administration of Exogenous Mutant Protein

Rather than using gene editing or gene therapy to correct a disease-causing point mutation, the approach provided herein can disrupt the misfolding process itself. For example, gene editing or gene therapy to create conversion-incompetent α-syn could reduce MSA propagation in vivo. Previous data have shown that α-synuclein prions from MSA samples could not replicate in cells expressing point mutations responsible for inherited Parkinson's disease (Woerman et al. Proc Natl Acad Sci USA 2018: 115 (2):409-14).

Inhibition of protein misfolding (and therefore treatment of disease associated with the misfolded protein) may be performed by gene editing, gene therapy, or administration of an exogenous protein, as described herein.

In some embodiments, gene editing via CRISPR prime editing relies on a reverse transcriptase (RT) from the Moloney murine leukemia virus (M-MLV) fused to the Cas9 nickase, which is directed to the gene of interest by a prime editing guide RNA (pegRNA) (24). After creating a nick in the protospacer adjacent motif (PAM) sequence-containing DNA strand, the pegRNA, which contains both a primer binding site and a template for RT, facilitates insertion of the desired mutation into the coding DNA strand (Anzalone et al. Nature 2019; 576 (7785):149-57). The second-generation prime editor (PE2) contains five mutations in M-MLV RT that increase editing efficiency. In the third generation (PE3), the PE2 nickase uses an sgRNA to insert a second nick in the non-edited DNA strand, which increases editing efficiency and reduces insertion/deletion (indel) byproducts by directing mismatch repair to the non-edited strand. Finally, PE3b expands on this optimization by adding spacers to the sgRNA to match the edited strand, which prevents nicking of the non-edited strand prior to completion of PAM strand editing, further minimizing indels (Anzalone 2019 and Anzalone et al. Nat Biotechnol. 2020; 38 (7):824-44). The incorporation of extension sequences into both guide RNAs substantially reduces previous PAM sequence constrictions, minimizing a known limitation to using base editing as a therapeutic strategy (Rees and Liu Nat Rev Genet 2018; 19 (12):770-88.; Anzalone 2019).

An alternative approach, when dominant-negative interference with protein misfolding occurs, AAVs or other similar vectors or delivery methods can be used to insert an exogenous copy of the mutant gene via gene therapy. Rather than edit the endogenous gene to prevent misfolding, dominant negative inhibition via the mutant protein will inhibit extension of the misfolded protein amyloid. There are several known single residues differences in PrP that contribute to species barriers. For example, dogs are protected from developing any prion disease due to a single amino acid substitution in their protein sequence. This barrier works via dominant-negative inhibition to prevent disease transmission. In examples such as this, inserting the exogenous mutant gene would exert the same effect as the gene editing or gene therapy approach described above.

Further, in some embodiments, a mutant protein as described herein may be exogenously provided to the patient. In some embodiments, the mutant protein is generated in vitro through standard biological methods and then administered to the patient. This mutant protein can then inhibit misfolding of the endogenously expressed protein. Such an approach would have advantages to avoid alterations of the patient's genome and allow for a reversible therapy, wherein the mutant protein would be biodegraded in the body via standard metabolic pathways.

In some embodiments, a method to treat a neurodegenerative disease comprises administering an exogenous mutant protein to a subject in need thereof, wherein the neurodegenerative disease is the result of protein misfolding and aggregate formation. In some embodiments, the exogenous mutant protein comprises one or more substitutions in the amino acid sequence of the protein so as to inhibit said protein from misfolding. In some embodiments, the substitution creates a change in the charge of the protein, substitutes a larger or smaller amino acid as compared to the wild type amino acid in the same position, disrupts a salt bridge, changes conformation and/or disrupts zipper formation. In some embodiments, the substitution is the result of substituting one amino acid for a larger amino acid.

In some embodiments, a method to treat a neurodegenerative disease comprises introducing a mutation in vivo in the gene of the protein that is misfolding in a subject in need thereof, wherein the neurodegenerative disease is the result of protein misfolding and aggregate formation. In some embodiments, the mutation is introduced via gene editing or gene therapy. In some embodiments, the mutation results in one or more substitutions in the amino acid sequence of the protein to inhibit said protein from misfolding.

In some embodiments, a method to treat a neurodegenerative disease comprises administering an exogenous mutant protein to a subject in need thereof, wherein the neurodegenerative disease is the result of protein misfolding and aggregate formation, wherein the exogenous mutant protein comprises one or more substitutions in the amino acid sequence of the protein so as to inhibit said protein from misfolding.

### Methods of Diagnosis of Neurodegenerative Diseases

In some embodiments, a panel of cell lines may be used to diagnose a neurodegenerative disease. As symptoms of various neurodegenerative diseases may overlap, diagnosis of a specific disorder can lead to therapy for the proper disease. This therapy could be a targeted therapy such as gene editing, gene therapy, or administration of an exogenous mutant protein as described herein. In some embodiments, a diagnosis of a specific disorder can avoid treatment of the patient with an agent that is unlikely to improve their disorder. In some embodiments, a diagnosis of a specific disorder can allow for treatment of the subject at an earlier stage of the disorder and allow for a better clinical outcome. In some embodiments, a negative diagnosis can allow the subject and their clinician to evaluate other disorders that might be causing the subject's symptoms.

A method to diagnose a neurodegenerative disease comprises adding a sample of misfolded protein aggregate from a patient to a collection of cell lines, wherein each cell line expresses a different protein of interest tagged with a reporter. The tagged protein may be any of those described herein that prevent misfolding/aggregation. However, this approach could be used with mutations of other proteins that have the capacity to misfold.

In some embodiments, the protein of interest contains one or more mutations, wherein the protein of interest in at least one cell line that prevents aggregation of the tagged protein indicates the neurodegenerative disease of the patient. In some embodiments, the tagged protein of interest expressed in multiple cell lines comprised in the panel that lack aggregation of the reporter indicates the neurodegenerative disease of the patient. The tagged protein may be any of those described herein for tau or α-synuclein that prevent misfolding/aggregation. However, this approach could be used with mutations of other proteins that have the capacity to misfold.

As used herein, diagnosis of a neurodegenerative disease includes diagnosis of a disease based on the shape, or strain, that a specific protein misfolds into in the disease state. Notably, a single protein can give rise to multiple diseases. For example, α-synuclein misfolding can cause Parkinson's disease, Lewy body dementia, or multiple system atrophy. As a result, the specific protective mutation needed to treat each disease may differ, based upon the strategy needed to interfere with α-synuclein misfolding into a particular shape. In this way, we describe herein a personalized medicine approach to diagnosing and treating neurodegenerative disorders in which the diagnosis is used to inform the specific protective mutations introduced using one of the described approaches In some embodiments, a diagnostic assay for “a neurodegenerative disease” can narrow diagnosis to a group of disorders that may be caused by misfolding of the same protein and/or can narrow diagnosis to a group of disorders that may be treated by the same gene therapy, gene editing, or administration of protective proteins, as described herein.

In some embodiments, the same protein of interest may be capable of blocking misfolding or aggregation induced by samples from patients displaying a variety of different symptoms. In some embodiments, the present diagnostic method can provide a more personalized diagnosis for a patient that is based not just on disease symptoms but also based on identifying the misfolding or protein aggregation underlying the patient's symptoms and/or identifying how this misfolding or protein aggregation can be inhibited.

A method to diagnose a neurodegenerative disease can comprise (1) adding a sample of misfolded protein aggregate from a patient to a panel of cell lines, wherein each cell line expresses a different tagged protein of interest comprising a reporter, wherein the protein of interest contains one or more mutations; and (b) measuring the amount of aggregation of the reporter in each cell line comprised in the panel, wherein the tagged protein of interest expressed in each cell line that lacks aggregation of the reporter indicates the neurodegenerative disease of the patient. In some embodiments, the protein of interest comprised in one or more cell line in the panel is a mutant protein that inhibits aggregation of a protein associated with one or more neurodegenerative diseases. In some embodiments, the protein of interest comprised in one or more cell line in the panel is a protein associated with one or more neurodegenerative diseases.

In some embodiments, the sample is obtained from a biopsy, nasal swab, skin, cerebral spinal fluid, and/or plasma samples. In some embodiments, the reporter is a fluorescent protein. In some embodiments, the method further comprises treating said patient for said neurological disorder.

In some embodiments, one or more cell line in a panel of cell lines is a transformed cell line, optionally wherein the transformed cell line is chosen from NIH 3T3, HeLa, PC12, SH-SY5Y, or HEK293T cells

In some embodiments, the patient is believed to have a neurodegenerative disease, but the disease has not been adequately identified or diagnosed. In some embodiments, the neurodegenerative disease comprises tau-related diseases (or tauopathies), including, but not limited to, Alzheimer's disease (in which the proteins AB, ApoE, and TMEM106B also contribute to the disease and may be targeted using the same approach), progressive supranuclear palsy, chronic traumatic encephalopathy, Pick's disease, argyrophilic grain disease, and corticobasal degeneration: α-synuclein-related diseases (or synucleinopathies), including, but not limited to, Parkinson's disease, multiple system atrophy (MSA), Lewy body diseases, and Parkinson's disease with dementia: Huntington's disease (caused by mutant Htt), amyotrophic lateral sclerosis (ALS: caused by TDP-43 and/or SOD1), diabetes, prion protein (PrP)-related diseases, including but not limited to, Creutzfeldt-Jakob disease, fatal familial insomnia, and Gerstmann-Sträussler-Scheinker disease.

In some embodiments, the neurodegenerative disease is a:
tau-related disease, optionally wherein the tau-related disease is Alzheimer's disease, progressive supranuclear palsy, chronic traumatic encephalopathy, Pick's disease, argyrophilic grain disease, or corticobasal degeneration;
α-synuclein-related disease, optionally wherein the α-synuclein-related disease is Parkinson's disease, multiple system atrophy (MSA), Lewy body diseases, and Parkinson's disease with dementia: Huntington's disease, amyotrophic lateral sclerosis, or diabetes; or
prion protein (PrP)-related disease, optionally wherein the PrP-related disease is Creutzfeldt-Jakob disease, fatal familial insomnia, or Gerstmann-Sträussler-Scheinker disease.

### EXAMPLES

### Example 1. Development of Cell Lines to Investigate Disruption of Protein Misfolding in MSA

Multiple system atrophy (MSA) is one of several prion neurodegenerative disorders for which there are no disease-modifying therapeutics available. Among these diseases, evidence supporting the role of the prion disease mechanism is strongest for the protein α-synuclein (α-syn) in MSA. Novel HEK293T bioreporter cell lines expressing α-syn-YFP, as well as transgenic (Tg) mice expressing human α-syn (Prusiner et al. Proc Natl Acad Sci USA 2015; 112 (38):E5308-E17: Woerman A L et al. PLOS Pathogens 2020; 16 (2):e1008222; and Woerman et al. Acta Neuropathol 2018; 135:49-63) have been described. α-syn aggregates isolated from MSA patient samples induce α-syn misfolding in both model systems and transmit terminal disease to mice. Moreover, MSA serially propagates in both cells and mice, as well as between the two model systems, consistent with the prion mechanism (Woerman et al. Proc Natl Acad Sci USA 2015; 112 (35):E4949-E58; and Woerman et al. Proc Natl Acad Sci USA 2018; 115 (2):409-14).

To interfere with prion propagation, several groups have proposed knocking down α-syn to reduce the amount of substrate available for templating, which would slow or halt fibril growth. However, because α-syn promotes SNARE complex assembly in the presynaptic terminal (Burré et al. Science 2010; 329 (5999):1663-7), this strategy may result in profound loss-of-function deficits. Additionally, α-syn knockout mice exhibit altered synaptic morphology and function (Cabin et al. J Neurosci 2002; 22:8797-807), and the loss of mouse α-syn can exacerbate disease in some models of synucleinopathy (Cabin et al. Neurobiol Aging 2005; 26:25-35). These data suggest that reducing α-syn expression may cause loss-of-function deficits in MSA patients. Alternatively, it has been shown that MSA cannot propagate in either cells or mice expressing α-syn with the E46K mutation (Table 2; Woerman et al. Proc Natl Acad Sci USA 2018: 115 (2):409-14). These findings resulted in the identification of a novel therapeutic strategy that employs non-pathogenic α-syn mutations to interfere with the aberrant self-templating process observed in protein misfolding disorders. However, the E46K mutation causes PD, and therefore does not represent a viable pathway to the clinic.

TABLE 2
Disease onset in E46K mice following intracranial injection

TgM47^{+/-} mice^{a}

| Sample | Inc^{b} | A/I^{c} |
|---|---|---|
| None | >545 dpi | 0/8 |
| E46K fibrils | 286 ± 122 | 8/8 |
| C9^{d} | >475 dpi | 0/8 |
| C17^{d} | >475 dpi | 0/8 |
| MSA5^{e} | >475 dpi | 0/8 |
| MSA14^{e} | >475 dpi | 0/8 |
| MSA17^{e} | >475 dpi | 0/8 |

^{a}TgM47+/− mice express E46K human α-syn7
^{b}Incubation period in days (mean ± SD) to onset of first neurological sign
^{c}Number affected (A)/number injected (I)
^{d}Samples from two different control patients
^{e}Samples from three different MSA patients

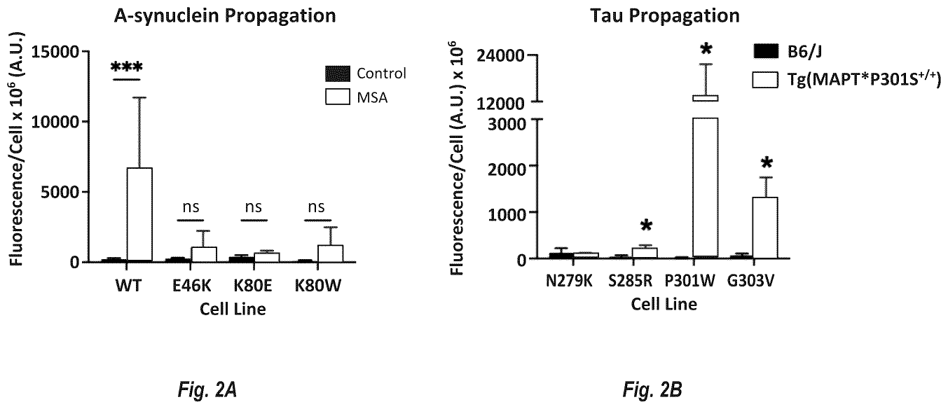

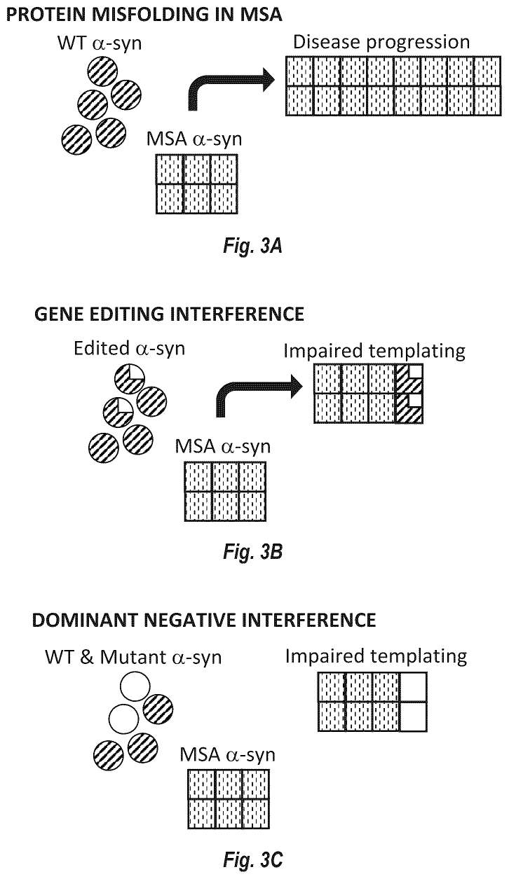

Hypothesizing that the effects of the E46K mutation are due to disruption of a key salt bridge between residues E46 and K80, which stabilizes the Greek key conformation that α-syn adopts in MSA patients, cells were built expressing α-syn140*K80E-YFP and α-syn140*K80W-YFP. These non-pathogenic mutations are incapable of forming a salt bridge with residue E46, and as a result, they also block MSA propagation in vitro (FIG. 2A).

This method allows further use of a gene editing technique, e.g., prime editing, to generate non-convertible α-syn as for MSA. For example, adeno-associated virus (AAV)-mediated CRISPR prime editing can be used to insert the K80E or K80W mutations into WT human α-syn to prevent MSA propagation in cells and mice so as to establish proof-of-concept for this novel gene editing strategy. Notably, this is the first application of gene editing to treat a disorder that is not known to be caused by a pathogenic mutation. To date, gene editing has only been explored as a therapeutic strategy to correct disease-causing point mutations. As no pathogenic point mutations have been identified in MSA patients, this innovative approach greatly expands the scope of diseases one can target with prime editing.

Cell lines used in these experiments were prepared using standard general molecular biology methods, and these same cell lines can be used for diagnostic methods. Gene sequences fused with YFP at the C-terminus were synthesized and introduced into the pcDNA3.1 (+) expression vector. The α-synuclein-YFP constructs were subcloned into the pIRESpuro3 vector (Clontech) using EcoRI (5′) and NotI (3′). HEK293T cells were cultured in DMEM supplemented with 10% fetal bovine serum, 50 units/mL penicillin, and 50 μg/mL streptomycin. Cultures were maintained in a humidified atmosphere of 5% CO₂ at 37° C. Cells plated in DMEM were transfected using Lipofectamine 2000. Stable cells were selected in DMEM containing 1 μg/mL puromycin (Thermo Fisher). Monoclonal lines were generated by limiting dilution of polyclonal cell populations in 384-well plates. To test samples in the cell assay, protein aggregates are isolated from 10% brain homogenates prepared in 1×DPBS using a sodium phosphotungstic acid (PTA) precipitation. In this protocol, 10% brain homogenate was incubated in 2% sarkosyl and 0.5% benzonase at 37° C. with constant agitation in an orbital shaker for 2 h. PTA was dissolved in ddH2O, and the pH was adjusted to 7.0. PTA was added to the solution to a final concentration of 2%, which was then incubated overnight in the same conditions. The sample was centrifuged at 16,000×g for 30 minutes at room temperature, and the supernatant was removed. The resulting pellet was resuspended in 2% sarkosyl in PBS and 2% PTA in ddH2O, pH 7.0. The sample was again incubated for at least 1 h prior to a second centrifugation. The supernatant was again removed, and the pellet was resuspended in DPBS using 10% of the initial starting volume. The cells are plated in a 384-well plate with black polystyrene walls at a density sufficient to reach 70% confluency in 4 days with 0.012 μg Hoechst 33342. Plates are incubated at 37° C. for 2-4 hours to allow the cells to adhere to the plate. Lipofectamine 2000 is added to each PTA-precipitated sample further diluted in DPBS, and the mixture is incubated at room temperature for 1.5 hours. OptiMEM was added to each sample prior to plating the patient sample in 6 replicate wells. Plates were then incubated at 37° C. in a humidified atmosphere of 5% CO₂ for 4 days before imaging on an automated fluorescent microscope.

To analyze effects, images of both the DAPI and FITC channels are collected from 5 different regions in each well. The images were analyzed using an algorithm developed to quantify the size and fluorescence intensity of intracellular aggregates only in live cells.

### Example 2. Example 2. Gene Editing of Tau

Gene editing to interfere with protein misfolding was also used to reduce tau propagation in vitro and in vivo. CRISPR prime editing (as described in Anzalone 2019 and Anzalone 2020) was used to edit WT tau to contain mutations that interfere with protein misfolding in both cell cultures and transgenic mice. Cells expressing tauRD-YFP with either the N279K or S285R mutations block propagation of tau aggregates isolated from Tg (MAPT*P301S^{+/+}) mouse samples (FIG. 2).

To further establish proof-of-concept, guide RNAs will be generated to edit WT tau-YFP cells to contain either the N279K or S285R mutations. This will involve identifying the guide RNA sequences that exhibit the greatest reduction in tau misfolding while also minimizing insertion and deletion (indels) byproducts, as determined by high-throughput sequencing of lysates from edited cells. These guide RNAs will then be inserted into AAV9 constructs. Approximately 4×10⁴ viral genomes of the two AAVs will be injected into the lateral ventricle of 6-week-old Tg (MAPT*P301S^{+/+}) mice, and the ability of gene editing to interfere with disease onset in the animals will be determined. Following the onset of neurological signs, brains from terminal animals will be analyzed for editing efficiency, indel byproducts, and neuropathological lesions in the brain.

### Example 3. In Vitro Models of PSP and CBD Tau

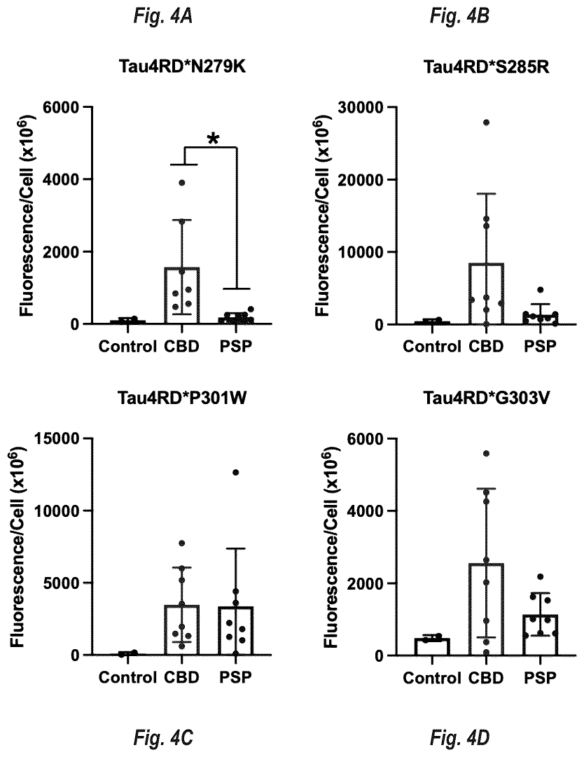

HEK293T Tau4RD-YFP cells that selectively propagate CBD tau were generated and cells that propagate PSP will be similarly generated. 4 mutant cell lines that express tau residues 244-380 (the templating region in PSP and CBD aggregates) tagged with YFP were created. These cell lines contain the mutations N279K, S285R, P301W, or G303V. These mutations were selected using structural analysis to predict which PSP-causing mutations were likely to disrupt tau misfolding into the reported structures for CBD tau (Zhang W et al. Nature 2020; 580 (7802):283-7). The one exception is P301W; P301L/S mutations are known to cause PSP but the protein was mutated to a larger residue to exert a stronger effect on protein folding. After isolating tau aggregates from 2 control, 8 CBD, and 8 PSP patient samples, the protein pellets were incubated with all 4 of the new cell lines for 4 days before imaging and analyzing using the Molecular Devices ImageXpress Micro XLS (FIG. 4).

Unexpectedly, it was found that both the N279K and S285R mutations, which cause PSP, inhibited PSP tau misfolding but supported propagation of CBD tau (FIGS. 4A & B). In comparison, the PSP and CBD samples propagate in both the P301W and G303V cell lines (FIGS. 4C & D), with particularly robust infection observed in the P301W cells. These data identified two different mutations that interfere with PSP tau misfolding.

The misfolded conformation of tau in PSP patient samples was resolved using cryo-electron microscopy (Shi Y et al. Nature 2021; 598 (7880):359-63). These data were not available when mutations were initially selected to test in the cell assay but have since allowed the identity of new mutations that can be used to block CBD infection while also supporting PSP propagation in vitro. Two such mutations are the K317H and D358E mutations. Mutating the aspartic acid to a larger, polar residue (like glutamic acid) should disrupt both the salt bridge and an anionic binding pocket in CBD. However, because this residue projects out from the PSP structure, it is hypothesized that no effect on tau misfolding into the PSP conformation.

### Example 3. Example 4. MSA Prions Transmit Neurological Disease to Mice Expressing Wild-Type Human α-Synuclein

In multiple system atrophy (MSA), the protein α-synuclein misfolds into a prion conformation that self-templates and causes progressive neurodegeneration. While many point mutations in the α-synuclein gene, SNCA, have been identified as the cause of heritable Parkinson's disease (PD), none have been identified as causing MSA. To examine whether MSA prions can transmit disease to mice expressing wild-type (WT) human α-synuclein, transgenic (Tg) mice denoted TgM20^{+/-} were inoculated with brain homogenates prepared from six different deceased MSA patients. All six samples transmitted CNS disease to the mice, with an average incubation period of ˜280 days. Interestingly, TgM20^{+/-} female mice developed disease >60 days earlier than their male counterparts. Brains from terminal mice contained phosphorylated α-synuclein throughout the hindbrain, consistent with the distribution of α-synuclein inclusions in MSA patients. Additionally, using α-syn-YFP cell lines, α-synuclein prions were detected in brain homogenates prepared from terminal mice that retained MSA strain properties. The studies described here are the first to show that MSA prions transmit neurological disease to mice expressing WT SNCA and that the rate of transmission is sex dependent.

By comparison, TgM20^{+/-} mice inoculated with WT preformed fibrils (PFFs) developed severe neurological disease in ˜210 days and exhibited robust α-synuclein neuropathology in both limbic regions and the hindbrain. Brain homogenates from these animals exhibited biological activities that are distinct from those found in MSA-inoculated mice when tested in the α-syn-YFP cell lines. Differences between brains from MSA-inoculated and WT PFF-inoculated mice potentially argue that α-synuclein prions from MSA patients are distinct from the PFF inocula and that PFFs do not replicate MSA strain biology.

Multiple system atrophy (MSA) is a progressive, invariably fatal neurodegenerative disease. While MSA is neuropathologically defined by the presence of glial cytoplasmic inclusions (GCIs) in oligodendrocytes [10, 30], the contribution of neuronal cytoplasmic inclusions to the disease has been increasingly recognized and investigated [34]. In 1998, Spillantini et al. first identified the protein α-synuclein as a major component of GCIs [30]: the previous year, α-synuclein was reported in Lewy bodies from the brains of Parkinson's disease (PD) patients [31]. These findings linked the two disorders as synucleinopathies.

Following these discoveries, the transmissibility of human MSA brain homogenates to transgenic (Tg) mice was identified [35]. Watts et al. used Tg mice that express human α-synuclein carrying the A53T mutation, which were designated TgM83^{+/-} mice. Brain homogenates prepared from two different MSA patient samples were intracranially inoculated into the TgM83^{+/-} mice and were found to induce neurological disease with an incubation period of ˜120 days. Subsequent studies testing a total of 17 homogenates prepared from 14 different MSA patients were collected from 3 different continents. Consistent with earlier findings, all MSA samples transmitted disease to the mice [27]. This work supported that α-synuclein becomes a prion in MSA patients.

It was subsequently demonstrated that α-synuclein prions in MSA samples are resistant to inactivation by formalin fixation, can transmit disease to mice following exposure to contaminated stainless steel wires (modeling iatrogenic transmission from surgical instruments), and can spread from the periphery into the brain following extraneural challenge in mice [37]. Moreover, MSA prions can propagate among multiple hosts, including two different Tg mouse models and cultured cells [36, 38].

While transmission of MSA prions from brains of patients was readily accomplished using Tg mice expressing mutant human α-synuclein [27], transmission to non-Tg mice has failed to induce neurological disease. Notably, species barriers in prion diseases are thought to arise from differences in the amino acid sequences of the prion protein (PrP). For example, of the 253 amino acids in PrP, the human and mouse sequences differ by 28 amino acids [32]. Similarly, among the 140 amino acids in α-synuclein, the human and mouse amino acid sequences differ at 7 residues, creating a species barrier that prevents MSA from propagating using mouse α-synuclein as substrate [21]. While these findings were used to argue that MSA is not a prion disease, they are consistent with several decades of research on Creutzfeldt-Jakob disease (CJD) prions showing that a species barrier prevents disease transmission to non-Tg mice. Efficient CJD transmission requires the use of Tg animals that express either human or human-mouse chimeric PrPs that do not prevent the species barrier from interfering with PrP misfolding [14, 32]. In contrast, the Tg mouse models used for MSA studies typically express human α-synuclein with the A53T mutation. This particular mutation was first identified 25 years ago in the Contursi kindred of PD patients [26], and as a result, it is commonly used in rodent models of synucleinopathy. The absence of any mutation in the human α-synuclein gene (SNCA) causing familial MSA has raised concerns about whether the TgM83^{+/-} mice are a relevant model for research on MSA.

In comparison with the TgM83^{+/-} mouse model, the TgM20^{+/-} mouse line expresses WT human α-synuclein [8]. In 2019, Dhillon et al. tested the ability of MSA patient samples to transmit disease to the TgM20^{+/-} mice by inoculating postnatal day 0 (P0) pups with lysates prepared from MSA samples [5]. A remarkable lack of phosphorylated α-synuclein neuropathology was when brains from the mice were collected 5 months later. In contrast, MSA-inoculated TgM83^{+/-} mice exhibited robust neuropathological lesions, leading to the conclusion that MSA prions cannot transmit to TgM20^{+/-} mice over the same time course. More recently, Lloyd et al. inoculated adult TgM83^{+/-} and TgM20^{+/-} mice with brain homogenates from two MSA patient samples and assessed the presence of α-synuclein neuropathology either postmortem in TgM83^{+/-} mice or 6 months after injection in TgM20^{+/-} mice [19]. The MSA patient samples induced α-synuclein inclusions in the brains of both mouse models, although to a remarkably lower extent than in mice inoculated with recombinant WT preformed fibrils (PFFs). Both the PFFs and MSA patient samples induced less phosphorylated α-synuclein pathology in the TgM20^{+/-} mice compared to the terminal TgM83^{+/-} mice. Importantly, studies investigating the effect of the A53T mutation on α-synuclein fibrillization kinetics have shown that the mutation accelerates α-synuclein misfolding and fibrillization [3, 9, 22]. It was hypothesized that MSA transmission to TgM20^{+/-} mice occurs over a longer incubation period than transmission to TgM83^{+/-} mice.

Described herein are MSA prions that induce neurological disease characterized by hind limb clasping, hind limb paralysis, loss of grip strength, bradykinesia, circling, ataxia, and kyphosis in mice expressing WT human α-synuclein. TgM20^{+/-} mice inoculated with six different MSA patient samples developed neurological signs ˜280 days postinoculation (dpi), or 9.5 months, whereas control-inoculated mice remained asymptomatic through 475 dpi. Moreover, female TgM20^{+/-} mice developed disease more than 60 days earlier, on average, than male mice following MSA inoculation. Brains from terminal animals contained pathological deposits of α-synuclein in the hindbrain, particularly in the midbrain and pons, which is likely responsible for the progressive degeneration observed in the mice. Additionally, α-synuclein prions isolated from brain homogenates prepared from symptomatic mice propagated in α-syn140-YFP cells expressing WT, A53T, and A30P, A53T α-synuclein. However, as previously demonstrated using MSA patient samples [36], the mouse-passaged samples could not infect cells expressing the E46K mutation or α-syn*A53T truncated at residue 95. These findings show that MSA prions retain their strain properties when passaged in TgM20^{+/-} mice. Critically, with the exception of the longer incubation period, transmission of MSA patient samples to TgM83^{+/-} mice yields a similar neuropathological lesion profile and an identical infectivity profile in the α-syn140-YFP cells [36, 39]. These findings suggest that the A53T mutation in TgM83^{+/-} mice accelerates α-synuclein misfolding kinetics without altering α-synuclein biology.

In contrast, TgM20^{+/-} mice inoculated with recombinant WT PFFs developed neurological disease ˜210 days dpi, or 7 months. Brains from terminal mice contained robust neuropathological inclusions throughout the hindbrain that spread into several limbic brain regions. This difference in lesion profile suggests that the WT PFFs are composed of different α-synuclein strain(s) than those found in MSA patient samples. Consistent with this interpretation, insoluble α-synuclein aggregates isolated from TgM20^{+/-} mice inoculated with WT PFFs were capable of propagating in cells expressing α-syn*A53T-YFP with a truncation at residue 95. These findings demonstrate that WT PFFs exhibit critical differences in biological activity compared to MSA patient samples, and, therefore, should not be used to investigate MSA strain biology.

### A. Materials and Methods

A variety of materials and methods were used to evaluate MSA prions.

### Human Tissue Samples

Frozen brain tissue samples from neuropathologically confirmed cases of MSA were provided by the Parkinson's UK Brain Bank at Imperial College London and the Massachusetts Alzheimer's Disease Research Center. Tissue was used from the basal ganglia from four of the MSA patient samples and the substantia nigra and surrounding midbrain from two of the MSA patient samples. Control patient tissue was provided by Dr. Deborah Mash (Miami Brain Bank). Demographic information about samples used is presented in Table 3.

TABLE 3
Patient sample information

| | Neurological | Age at | | | |
|---|---|---|---|---|---|
| Patient | Disease | Death | Sex | Brain Region | Brain Bank |
| C9 | None | 62 | M | Putamen | University |
| | | | | | of Miami |
| C17 | None | 65 | M | Putamen | University |
| | | | | | of Miami |
| MSA5 | MSA | 60 | M | Basal ganglia | Parkinson's |
| | | | | | UK |
| MSA6 | MSA | 61 | F | Basal ganglia | Parkinson's |
| | | | | | UK |
| MSA13 | MSA | 75 | M | Substantia nigra | MADRC^{b} |
| MSA14 | MSA-C^{a} | 76 | M | Basal ganglia | MADRC^{b} |
| MSA16 | MSA-C^{a} | 61 | F | Basal ganglia | MADRC^{b} |
| MSA17 | MSA | 60 | M | Substantia nigra | MADRC^{b} |

^{a}Multiple system atrophy-cerebellar subtype
^{b}Massachusetts Alzheimer's Disease Research Center

### Mice

Animals were maintained in an AAALAC-accredited facility in compliance with the 8th edition of the Guide for the Care and Use of Laboratory Animals. All procedures used in this study were approved by the University of California San Francisco Institutional Animal Care and Use Committee. All animals were housed under ABSL-2 conditions in an environmentally controlled room (10-15 air changes per hour) at a temperature of 22.5° C.±1.4° C., a relative humidity of 45%±15%, and with a 12-hour light/dark cycle. Animals had free access to a Tekland diet from Envigo (Indianapolis, IN) and tap water. Mice were group housed unless an animal's health status necessitated individual housing. The B6;C3-Tg (Prnp-SNCA) 20Vle (referred to here as TgM20^{+/-}) mice [8] were kindly provided by Dr. Benoit Giasson (University of Florida).

### Inoculations

Fresh-frozen human tissue was used to create a 10% (wt/vol) homogenate using calcium- and magnesium-free 1× Dulbecco's phosphate buffered saline (DPBS) using the Omni Tissue Homogenizer (Omni International). Homogenates were diluted to 1% using 5% (wt/vol) bovine serum albumin in 1×DPBS. Recombinant WT α-synuclein (Sigma-Aldrich) was aggregated in 1×DPBS as previously described [38]. Recombinant WT PFFs were diluted in 1×DPBS to a final concentration of 1 mg/mL.

Eight-week-old TgM20^{+/-} mice were anesthetized with isoflurane prior to inoculation. Freehand inoculations were performed using 30 μL of the 1% brain homogenate or 1 mg/mL WT PFFs into the thalamus. Following inoculation, all mice were assessed twice each week for the onset of neurological signs based on standard diagnostic criteria for prion disease [2]. Uninoculated mice were euthanized at 546 days of age. Control-inoculated TgM20^{+/-} mice were euthanized 475 dpi. MSA-inoculated animals were euthanized 500 dpi or following the onset of progressive neurological signs. Following euthanasia, the brain was removed and bisected down the midline. The left hemisphere was frozen for reporter cell assays and biochemical analysis, and the right hemisphere was fixed in formalin for neuropathological assessment.

### Immunohistochemistry and Neuropathology

Formalin-fixed mouse half-brains were cut into four sections prior to processing through graded alcohols, clearing with xylene, infiltrating with paraffin, and embedding. Thin sections (8 μm) were cut, collected on slides, deparaffinized, and exposed to heat-mediated antigen retrieval with citrate buffer (0.1 M; pH 6) for 20 min. Slides were stained using the Thermo Fisher 480S Autostainer with 30 min blocking in 10% (vol/vol) normal goat serum and incubating with primary and secondary antibodies (2 h each). Primary antibody binding of phosphorylated (S129) α-synuclein (EP1536Y; 1:1,000; Abcam) and glial fibrillary acidic protein (GFAP; 1:500; Abcam) were detected using secondary antibodies conjugated to AlexaFluor 488 or 647 (1:500; Thermo Fisher Scientific). Slides were imaged using the Zeiss AxioScan.Z1 and were then analyzed using the ZEN Analysis software package (Zeiss). To quantify α-synuclein neuropathology, a pixel intensity threshold was determined using a positive control slide. Regions of interest were drawn around the caudoputamen (Cd), hippocampus (HC), piriform cortex and amygdala (PC), thalamus (Thal), hypothalamus (HTH), midbrain (Mid), and pons. The percentage of positively stained pixels in each region was determined and averaged across inoculation groups.

### α-Synuclein Prion Bioassay

Frozen brain tissue was used to make a 10% (wt/vol) brain homogenate in calcium- and magnesium-free 1×DPBS using the Omni Tissue Homogenizer with disposable soft tissue tips (Omni International). Aggregated protein was isolated from the homogenates using phosphotungstic acid (PTA; Sigma-Aldrich) as described [29, 40]. Isolated protein pellets were diluted 1:10 in 1×DPBS before testing in the α-synuclein prion bioassays. HEK293T parent cell line was obtained from ATCC (line CRL-3216). Transfection to establish α-syn-YFP cell lines was done using a low passage number. HEK293T cells expressing α-syn140-YFP (WT), α-syn140*E46K-YFP (E46K), α-syn140*A53T-YFP (A53T), α-syn140*A30P,A53T-YFP (A30P,A53T), α-syn140*E46K,A53T-YFP (E46K,A53T), and α-syn95*A53T-YFP (1-95) were generated as previously described; culture and bioassay conditions were also used as previously described [36]. Cells incubated with isolated protein pellets were imaged using the IN Cell Analyzer 6000 (GE Healthcare). DAPI and FITC images from five different regions in each well were analyzed using IN Cell Developer software with an algorithm designed to quantify intracellular aggregates, represented as total fluorescence per cell (×10³, arbitrary units [A.U.]). One measurement was generated for each well across the five regions imaged, and each sample was tested in six replicate wells.

### Immunoblotting

To visualize soluble α-synuclein in TgM20^{+/-} mouse brain homogenates, samples were clarified by centrifugation for 5 min at 1,000×g. The supernatant was collected, and total protein was measured using the bicinchoninic acid (BCA) assay (Pierce). A total of 2.5 μg total protein was prepared in 1× NuPAGE LDS loading buffer and boiled for 10 min. Samples were loaded onto a 10% Bis-Tris gel, and SDS-PAGE was performed using the MES buffer system. Protein was transferred to a polyvinylidene fluoride (PVDF) membrane. The membrane was fixed in 0.4% formalin for 30 min at room temperature prior to blocking in blocking buffer (5% [wt/vol] nonfat milk in 1× Tris-buffered saline containing 0.05% [vol/vol] Tween 20 [TBST]) for 30 min at room temperature. Blots were incubated with primary antibodies for phosphorylated α-synuclein (EP1535Y; 1:4,000; Abcam) and the loading control vinculin (1:10,000; Abcam) in blocking buffer overnight at 4° C. in a vacuum-sealed pouch. Membranes were washed three times with 1×TBST before incubating with horseradish peroxidase-conjugated goat anti-rabbit secondary antibody (1:10.000; Abcam) diluted in blocking buffer for 1 h at 4° C. in a vacuum-sealed pouch. After washing blots three times in 1×TBST, membranes were developed using the enhanced chemiluminescent detection system (Pierce) for exposure to X-ray film.

To detect insoluble α-synuclein, protein aggregates were isolated via PTA-precipitation, and resuspended pellets were diluted 1:5 in 1× NuPAGE LDS loading buffer and 1×DPBS. Samples were boiled for 20 min before loading on a 10% Bis-Tris gel. Using the protocol described above, PVDF membranes were probed for phosphorylated α-synuclein (EP1536Y; 1:4,000; Abcam).

### Statistical Analysis

Data are presented as mean±standard deviation. Data were analyzed using GraphPad Prism software. Kaplan-Meier curves and differences in incubation period for female and male mice were analyzed using a log-rank Mantel-Cox test. Analysis of data collected from the α-syn-YFP cell assays and neuropathology data comparing control-inoculated and PFF-inoculated samples was performed using a two-way ANOVA with a Tukey multiple comparison post hoc test. Cell assay and neuropathology data comparing control- and MSA-inoculated mice were analyzed using a two-way ANOVA with a Dunnett multiple comparison post hoc test. Neuropathology and cell assay data comparing MSA- and PFF-inoculated mice were analyzed using a two-way ANOVA with a Bonferroni multiple comparison post hoc test. Significance was determined with a P-value <0.05.

### Human Patient Neuropathology

Neuropathology assessment by the Parkinson's UK Brain Bank was performed using human tissue samples bisected down the midline. One hemisphere was fixed in 10% buffered formalin, and the other hemisphere was sliced coronally, photographed on a grid, and rapidly frozen. The fixed tissue blocks from 20 key brain regions were stained with hematoxylin and eosin (H&E) and Luxol fast blue (LFB). To diagnose and stage disease, appropriate blocks were stained with antibodies against α-synuclein, β-amyloid, tau, and p62. An MSA diagnosis was based on α-synuclein inclusions in oligodendrocytes [Alafuzoff et al (2009) Acta Neuropathol 117:635-652]

Neuropathology assessment by the Massachusetts Alzheimer's Disease Research Center (ADRC) Brain Bank was performed using human tissue samples bisected down the midline. One hemisphere was fixed in 10% (vol/vol) neutral buffered formalin and coronally sectioned, and the other hemisphere was coronally sectioned before rapid freezing. The fixed tissue was evaluated histologically using a set of blocked regions representative of a variety of neurodegenerative diseases. All blocks were stained with H&E and LFB. Selected blocks were used for immunohistochemical staining for α-synuclein, β-amyloid, and phosphorylated tau. A confirmed MSA diagnosis required the presence of glial cytoplasmic inclusions [Gilman et al (2008) Neurology 71:670-676].

### B. Incubation Times Following MSA Transmission to TgM20^{+/-} Mice are Sex Dependent

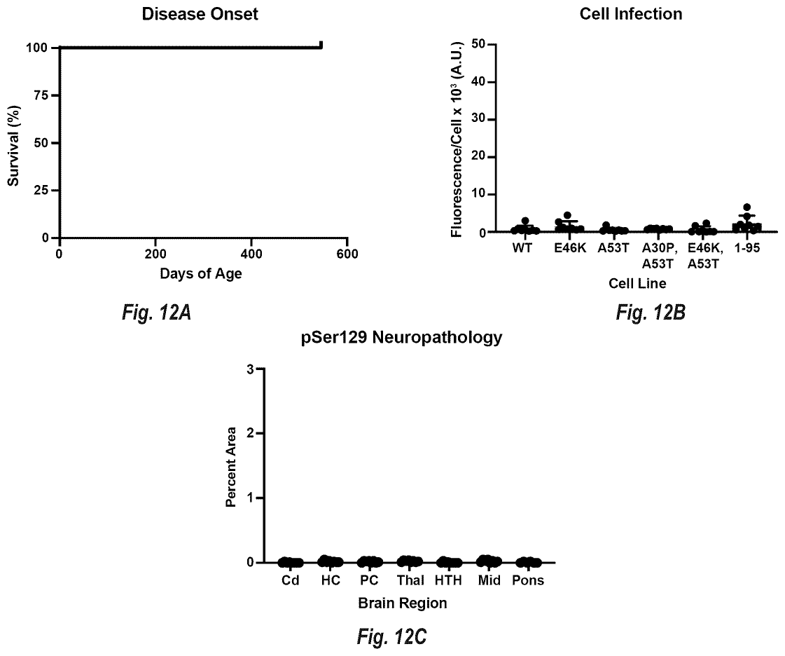

Initial studies using the TgM20 mouse model reported that neither the hemizygous nor the homozygous animals developed spontaneous neurological disease [5, 6, 8, 19]. To confirm this in a TgM20^{+/-} colony, hemizygous mice were assessed for neurological signs twice each week through 546 days (78 weeks) of age and found no indication of spontaneous synucleinopathy (FIG. 12A). Brains were dissected from the 546-day-old TgM20^{+/-} mice, flash-freezing one half (cut sagittally) and fixing the other half in formalin. The frozen brain tissue was homogenized and isolated protein aggregates by PTA precipitation. The resulting pellets were tested for the presence of α-synuclein prions using α-syn-YFP prion bioassays [36, 40]. None of the samples propagated in any of the cell lines tested (FIG. 12B). Immunostaining of the fixed half-brain for phosphorylated α-synuclein (EP1536Y primary antibody) showed an absence of α-synuclein prion inclusions in the brains of the aged mice (FIG. 12C). Together, these results confirmed that the TgM20^{+/-} mice do not develop spontaneous α-synuclein prion disease, eliminating a possible confounding factor in these studies.

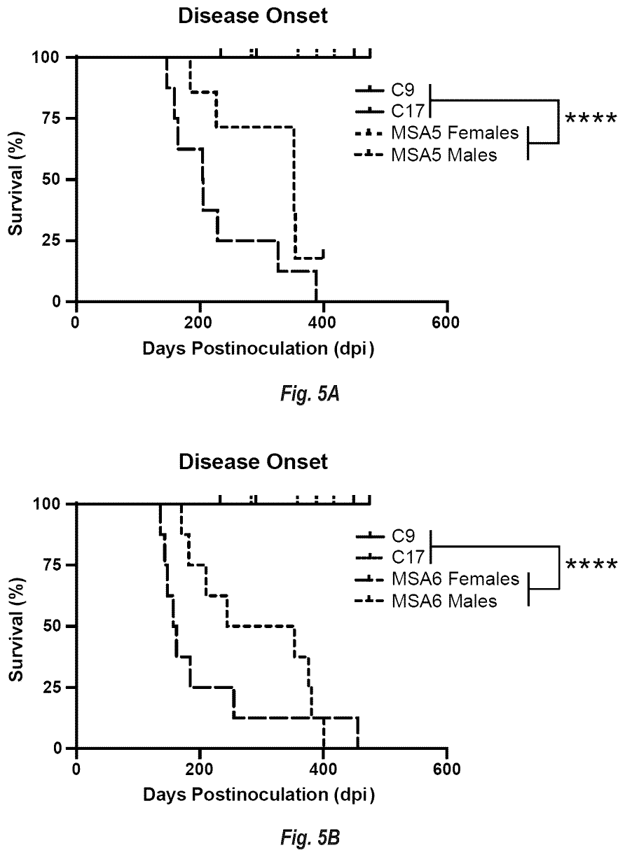

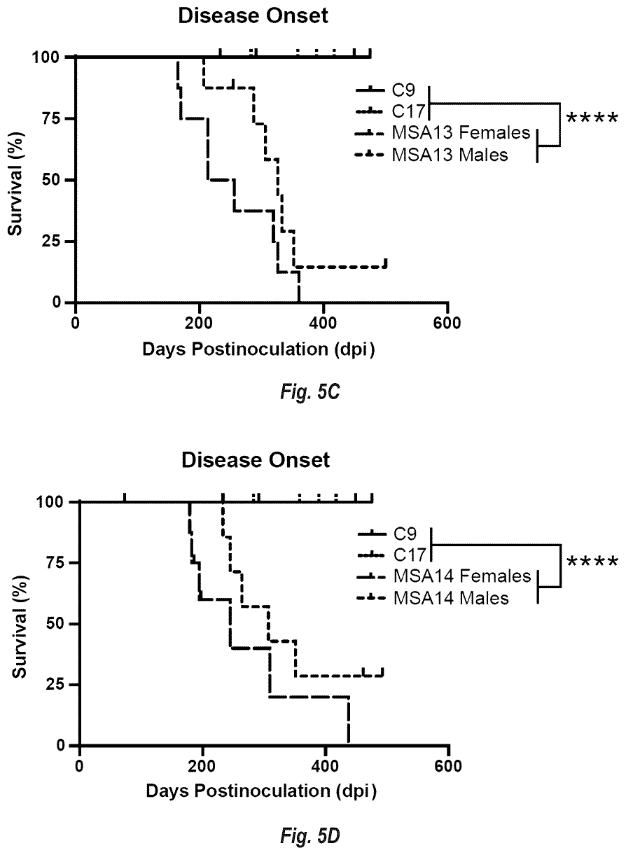

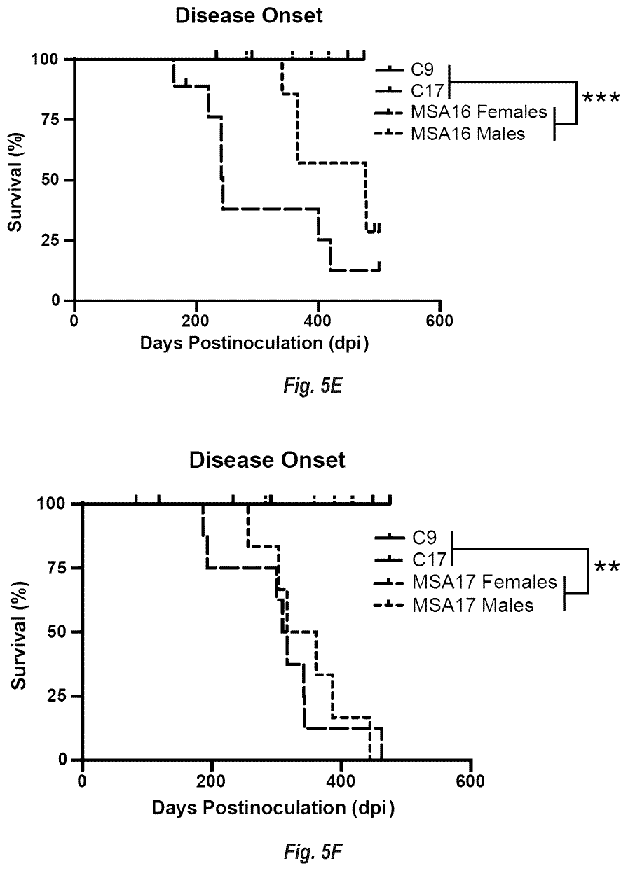

Recent studies by others demonstrated that TgM20^{+/-} mice develop α-synuclein neuropathology 6 months after inoculation with MSA prions from patient samples. The ability of MSA samples to induce neurological disease in TgM20^{+/-} mice has not been reported. To determine if MSA prions can transmit disease to TgM20^{+/-} mice, eight-week-old animals were inoculated with 30 μL of 1% brain homogenate prepared from either two control patient samples or six different MSA patient samples (FIGS. 5A-5H; Table 4).

TABLE 4
Incubation period and attack rates.

| | All Mice | | Female Mice | | Male Mice | | |
|---|---|---|---|---|---|---|---|
| | Incubation | | Incubation | | Incubation | | |
| | Period | | Period | | Period | | |
| | Avg ± SD | | Avg ± SD | | Avg ± SD | | |
| Sample | (dpi^{a}) | n/n₀^{b} | (dpi^{a}) | n/n₀^{b} | (dpi^{a}) | n/n₀^{b} | P Value |
| C9 | >475 | 0/8 | n.d. | | n.d. | | |
| C17 | >475 | 0/8 | n.d. | | n.d. | | |
| WT PFFs | 210 ± 37 | 7/7 | n.d. | | n.d. | | |
| MSA5 | 263 ± 93 | 14/15 | 227 ± 86 | 8/8 | 311 ± 85 | 6/7 | 0.08 |
| MSA6 | 247 ± 109 | 16/16 | 205 ± 108 | 8/8 | 290 ± 98 | 8/8 | 0.31 |
| MSA13 | 289 ± 88 | 14/16 | 253 ± 75 | 8/8 | 302 ± 52 | 6/8 | 0.18 |
| MSA14 | 287 ± 99 | 11/15 | 258 ± 101 | 6/7 | 280 ± 49 | 5/8 | 0.20 |
| MSA16 | 363 ± 117 | 12/16 | 275 ± 96 | 7/9 | 406 ± 67 | 5/7 | 0.13 |
| MSA17 | 323 ± 79 | 14/16 | 306 ± 88 | 8/8 | 344 ± 67 | 6/8 | 0.65 |
| MSA | 281 ± 93 | 81/94 | 254 ± 93 | 45/48 | 317 ± 80 | 36/46 | 0.0005 |
| (all) | | | | | | | |

^{a}Days postinfection;
^{b}Number of symptomatic animals (n)/number of mice inoculated (n₀)
n.d. Not done.

While the control-inoculated mice remained healthy through 475 dpi, MSA-inoculated animals developed progressive neurological signs 281±93 days postinoculation (dpi: P<0.001), indicating that MSA transmission to Tg mice requires expression of the human α-synuclein protein. Notably, this average incubation period is more than twice the length observed in the TgM83^{+/-} mouse model (˜120 dpi) [27]. While the TgM20^{+/-} mice actually express more human α-synuclein than the TgM83^{+/-} line [8], the A53T mutation is known to accelerate α-synuclein misfolding [3, 9, 22], which likely explains the decreased incubation periods observed in the inoculated TgM83^{+/-} mice.

While MSA appears to afflict roughly an equal number of men and women, recent work has shown that median survival for males is significantly shorter than for females. Survival from diagnosis is almost one year shorter for males compared to females [4]. To determine if similar sex-specific effects were present in these experiments, the onset of neurological signs in female and male TgM20^{+/-} mice inoculated with each MSA patient sample were compared (FIGS. 5A-5H: Table 4). Notably, the female mice developed disease faster than the male mice: however, the studies were underpowered to support statistical analysis within an inoculation group. When difference in incubation period across mice inoculated with all 6 MSA patient samples were compared, data showed that females developed disease >60 days before the males (Females: 254±93 dpi with 45 of 48 mice becoming symptomatic: Males: 317±80 dpi with 36 of 46 mice becoming symptomatic: P=0.0005). Importantly, both female and male mice developed the same neurological signs, despite this difference in incubation period. Efforts to determine why female TgM20^{+/-} mice develop disease earlier than male animals will be the focus of future studies.

### C. Symptomatic MSA-Inoculated TgM20^{+/-} Mice Develop Pathogenic α-Synuclein Inclusions

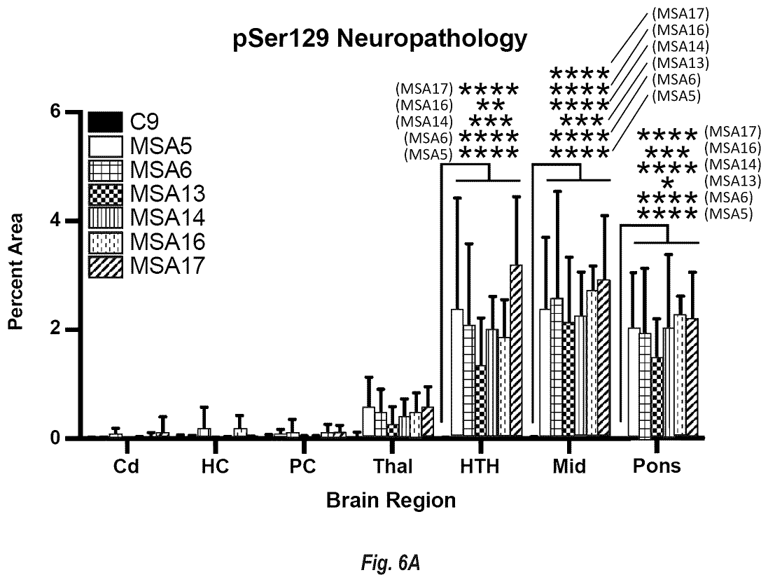

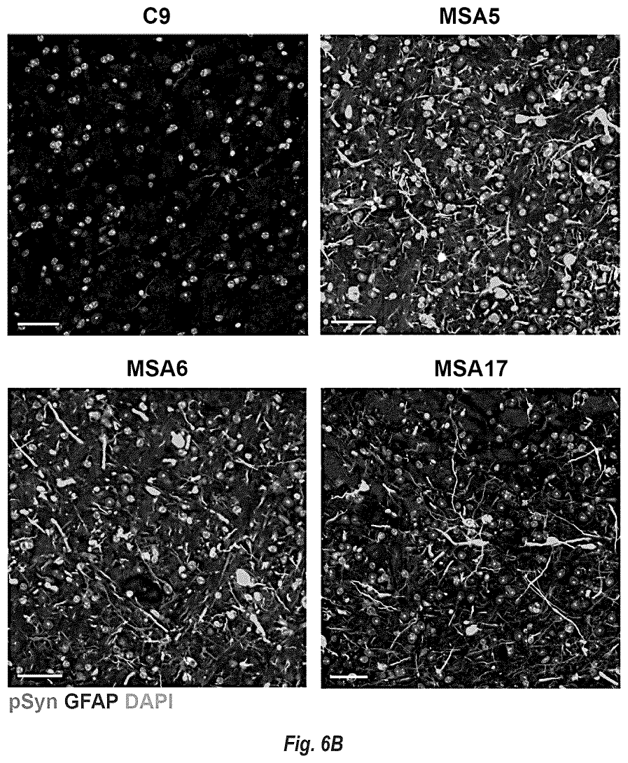

To determine the neurological changes driving the onset of disease in symptomatic MSA-inoculated TgM20^{+/-} mice, fixed half-brains were analyzed for the presence of phosphorylated α-synuclein inclusions (FIGS. 6A and 6B). The MSA patient samples induced significant neuropathology in the hypothalamus, midbrain, and pons (P<0.05), but the changes in the thalamus were not significant (FIG. 6A). By comparison, control-inoculated mice did not develop α-synuclein neuropathology. Consistent with the use of the Prnp promoter to express the SNCA transgene, TgM20^{+/-} mice appear to primarily develop neuronal inclusions that fill both the neurite and cell body. Representative images of immunostaining in the pons from mice inoculated with C9, MSA5, MSA6, and MSA17 patient samples also indicate an increase in GFAP immunostaining (astrogliosis) around the α-synuclein lesions in MSA-inoculated animals (FIG. 6B). The distribution of pathological lesions in these brain regions is consistent with the onset of overt motor signs that include hind limb clasping, difficulty rearing, weak grip strength, circling, and bradykinesia.

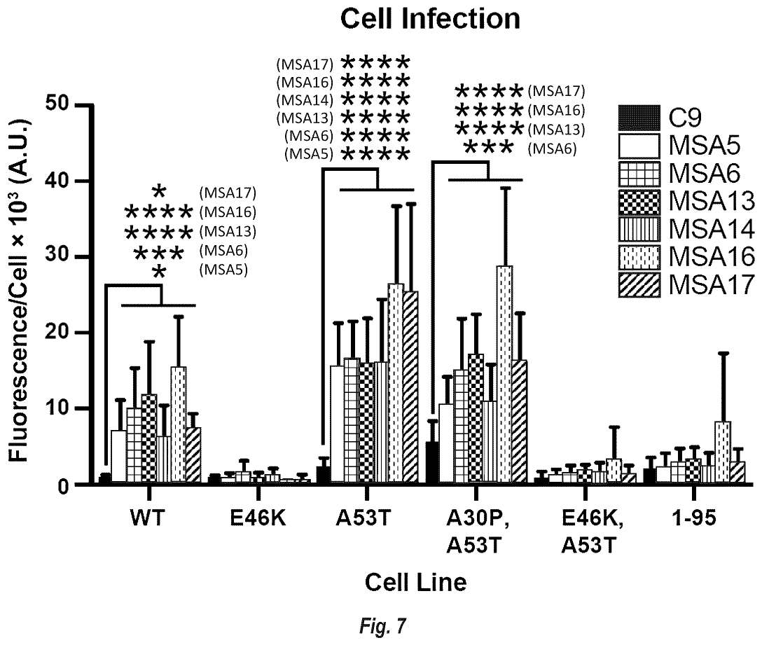

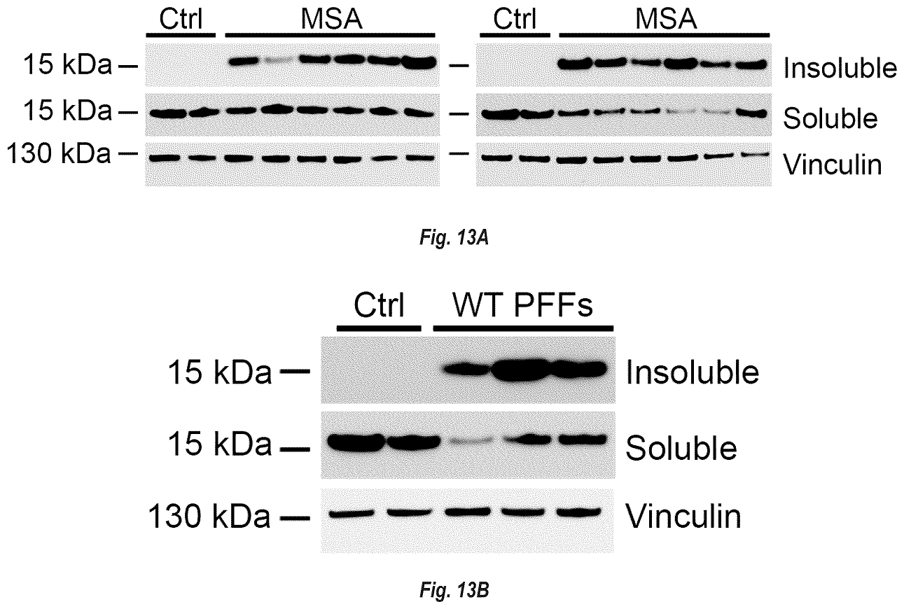

Using a cultured cell bioassay for α-synuclein prions, the levels of MSA prions were measured in the brains of inoculated TgM20^{+/-} mice (FIG. 7). Consistent with previous findings [36], mouse-passaged MSA samples infected α-syn-YFP cells expressing WT, A53T, or the double mutation A30P and A53T (P<0.05 for MSA samples compared to C9). Unsurprisingly, the human MSA prions could not propagate in cells expressing the E46K or the E46K and A53T double mutation. Notably, α-synuclein prions could not propagate in cells expressing α-syn*A53T truncated at residue 95 (1-95 sample, FIG. 7; P>0.05) either. These findings demonstrate that MSA α-synuclein prions retain their strain-specific properties during passaging in the TgM20^{+/-} mice, yielding similar findings to TgM83^{+/-} transmission studies [36, 38]. The presence of pathogenic α-synuclein aggregates in the terminal mouse brains was confirmed by probing for detergent-insoluble phosphorylated α-synuclein using Western blots (FIG. 13A). Control-inoculated mouse samples lacked insoluble α-synuclein while phosphorylated insoluble α-synuclein was present in MSA-inoculated samples (top blots). All mouse samples contained soluble phosphorylated α-synuclein (middle blots). Together, these results show that MSA prions transmit neurological disease and pathological inclusions to mice expressing WT human α-synuclein.

### D. TgM20^{+/-} Mice Inoculated with WT Preformed Fibrils (PFFs) Develop Artificial α-Synuclein Prions with Biological Activities Distinct from MSA Prions

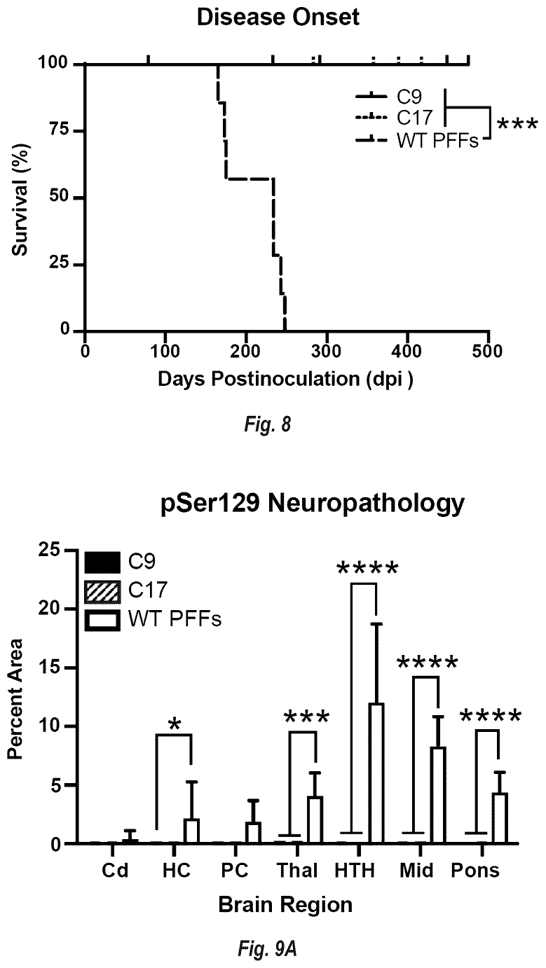

Using a α-syn-YFP cell-based assay, WT PFFs have been shown to exhibit a distinct biological activity compared to the α-synuclein prions isolated from MSA patient samples [36]. To determine if similar strain differences could be detected using mouse bioassay in TgM20^{+/-} animals, the ability of WT PFFs to induce neurological disease was tested in the mice. TgM20^{+/-} mice inoculated with 30 μL of 1% brain homogenate from two control patient samples (C9 and C17) remained asymptomatic through 475 dpi (FIG. 8). However, mice inoculated with 30 μg of WT PFFs developed progressive neurological signs 210±37 dpi (P<0.001). This incubation period is notably shorter than the ˜280 days following MSA inoculation: however, it is important to note that this difference may be due to differences in the titer of the inoculum and not the strain.

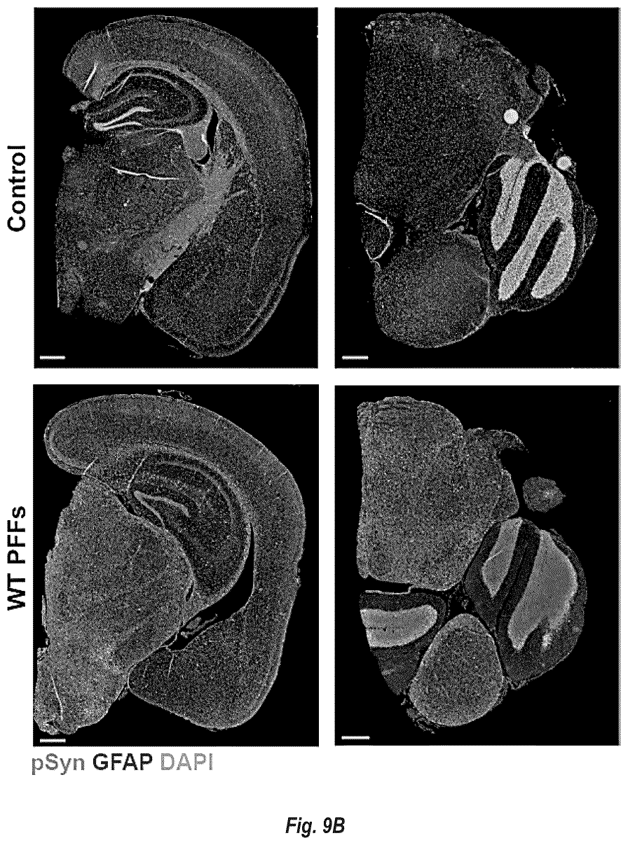

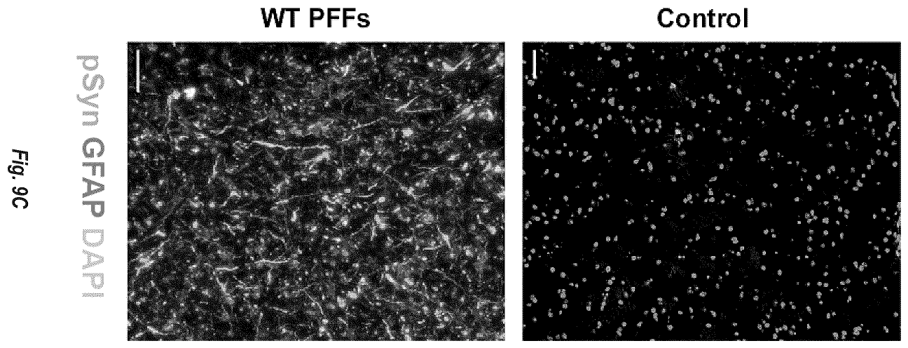

Fixed half-brains from the control-inoculated and WT PFF-inoculated TgM20^{+/-} mice were immunostained for phosphorylated α-synuclein and GFAP (FIGS. 9A-9C). WT PFFs induced robust and widespread neuropathology (FIG. 9a), with significant accumulation in the hippocampus (P<0.05), thalamus (P<0.001), hypothalamus (P<0.0001), midbrain (P<0.0001), and pons (P<0.0001). While α-synuclein pathology was observed in the piriform cortex & amygdala of some WT PFF-inoculated mice, it was not present in all and was, therefore, non-significant (P=0.1). An increase in GFAP staining was also found around these inclusions compared to control-inoculated animals, indicating that the α-synuclein inclusions coincide with astrogliosis in the mice (FIGS. 9B and 9C).

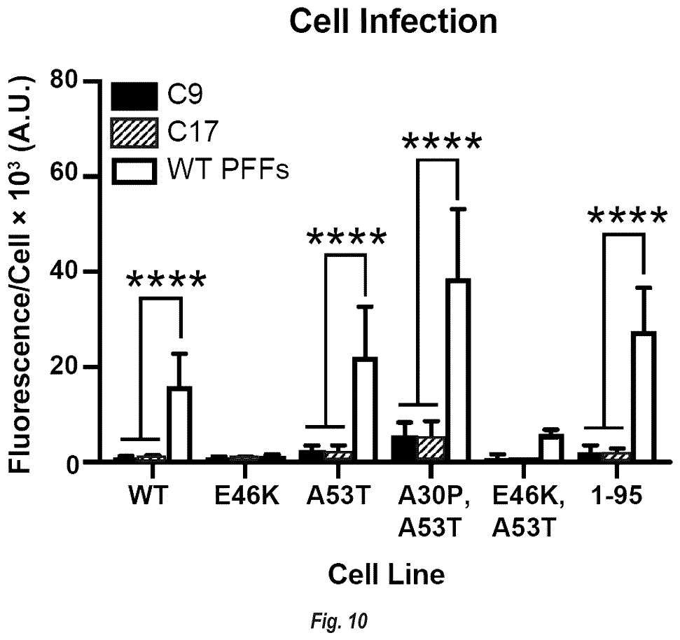

Next, frozen half-brains collected from the control- and PFF-inoculated mice in the α-syn-YFP cell lines were assessed for the presence of pathogenic α-synuclein (FIG. 10). The α-synuclein prions isolated from the PFF-inoculated animals infected cells expressing WT, A53T, and A30P,A53T α-synuclein as well as α-syn*A53T truncated at residue 95 (P<0.0001). Interestingly, WT PFFs have been shown to propagate in cell lines expressing E46K and E46K,A53T α-synuclein: however, their ability to do so is diminished compared to other cell lines [36]. WT PFF conformations that cannot use E46K α-synuclein as substrate selectively propagated in TgM20^{+/-} mice. In addition to cell infectivity studies, frozen brain samples were analyzed for the presence of insoluble phosphorylated α-synuclein (FIG. 13B). Samples from two control-inoculated animals did not contain insoluble α-synuclein, whereas samples from three PFF-inoculated mice did (top blot: EP1536Y primary antibody). In comparison, all five samples contained soluble phosphorylated α-synuclein (middle blot).

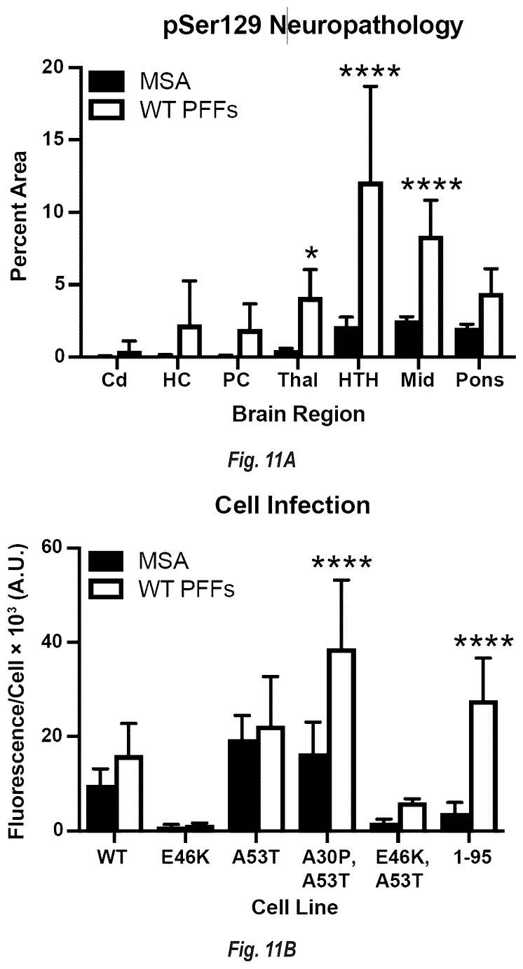

To determine if strain differences could be resolved between MSA-inoculated and WT PFF-inoculated TgM20+/− mice, the distribution of phosphorylated α-synuclein inclusions was compared between the two groups of mice (FIG. 11A). Here, the average measurement made across all MSA-inoculated mice was compared with the WT PFF-injected animals. Unlike the MSA inocula, WT PFFs induced α-synuclein inclusions in the hippocampus and piriform cortex and amygdala, but this change was not significantly different, while a significant increase in the intensity of α-synuclein staining in the thalamus (P<0.05), hypothalamus (P<0.0001), and midbrain (P<0.0001) was detected. Additionally, when cell infectivity data was compared between MSA- and PFF-inoculated mice, brains from PFF-inoculated mice propagated in the α-syn95*A53T-YFP cells while MSA-inoculated mouse samples did not (FIG. 11B; P<0.0001). Combined, these findings indicate that while WT PFFs are capable of inducing the formation of pathogenic α-synuclein prions in TgM20^{+/-} mice, the strain differences between MSA prions and WT PFFs result in distinct distributions of neuropathological inclusions and biological activities in a cell-based assay. Therefore, WT PFFs should not be used as an alternative to MSA patient samples to investigate MSA disease pathogenesis.

### E. Discussion

Over the past decade, α-synuclein prions in MSA patient samples have been shown to propagate in two different synucleinopathy mouse models expressing human α-synuclein with the A53T mutation [1, 5, 16, 27, 35-40]. These studies have been criticized for using animal models harboring a PD-causing SNCA mutation because no SNCA mutations have been identified in MSA patients. That MSA patient samples cannot induce neurological signs in mice expressing endogenous WT α-synuclein has been used as an argument against recognizing MSA as a prion disease. Here, for the first time, six different MSA patient samples collected from two different continents are shown to transmit neurological disease and induce α-synuclein prion formation in mice expressing WT human α-synuclein.

Initial MSA transmission experiments relied on the TgM83^{+/-} mouse model, which uses the Prnp promoter to express human α-synuclein with the A53T mutation [8]. Because the Prnp promoter limits α-synuclein expression to neurons in the mouse brain, terminal mice in these experiments developed neuronal inclusions. Though these neuronal α-synuclein deposits differ from the GCIs in oligodendrocytes [25], they are consistent with the presence of neuronal cytoplasmic inclusions in MSA patient samples [34]. With the goal of inducing glial pathology upon MSA transmission, Tg (SNCA*A53T^{+/+}) Nbm mice were inoculated with MSA patient samples [38]. The Tg (SNCA*A53T^{+/+}) Nbm line was generated using a P1 artificial chromosome containing the full human SNCA gene with its normal intron and exon structure as well as 35 kb of upstream regulatory elements [15]. As a result, transgene expression in these mice is much more widespread than in the TgM83^{+/-} animals. While MSA patient samples did not induce neurological signs in any of the inoculated Tg(SNCA*A53T^{+/+}) Nbm mice due to the lack of hindbrain pathology, they did propagate and form both neuronal and glial inclusions in the limbic system [38]. Given that MSA pathology in human patients is located in both glial and neuronal cells [12, 13, 24, 25], these findings were important for demonstrating the neuropathological hallmarks of MSA in vivo using this particular model. Moreover, MSA-inoculated Tg (SNCA*A53T^{+/+}) Nbm mouse brain samples were used for second passage experiments in TgM83^{+/-} mice, MSA prions can propagate between hosts while faithfully maintaining strain properties, as determined using α-syn-YFP cell lines [38].

Having replicated MSA pathology in vivo, MSA patient samples were tested to see if they can transmit disease to mice expressing WT human α-synuclein. The TgM20^{+/-} line were used for these experiments because it was generated using the same promoter and methods as the TgM83^{+/-} mice [8]. While differences in transgene expression or incorporation cannot be perfectly controlled, the TgM20^{+/-} line is the closest matched comparison to the TgM83^{+/-} mouse model, allowing for testing of the effect of host genotype on MSA α-synuclein prion transmission. Using a similar rationale, Dhillon et al. reported in 2019 that MSA inoculations in P0 TgM20^{+/-} mouse pups failed to induce α-synuclein neuropathology within 5 months of inoculation [5]. The authors selected the 5-month time point based on the average incubation periods reported for the TgM83^{+/-} mice (˜120 days). However, because the A53T mutation accelerates protein misfolding kinetics [3, 9, 22], MSA transmission to TgM20^{+/-} mice may require a longer incubation period. Consistent with this prediction, Lloyd et al. recently reported the presence of α-synuclein neuropathology in adult TgM20^{+/-} mice terminated 6 months after inoculation with MSA patient samples [19]. However, none of the mice in these studies developed neurological signs.

In the studies reported here, MSA patient samples transmit neurological disease to TgM20^{+/-} mice with an average incubation period of ˜280 days, or roughly 9.5 months (FIGS. 5A-5F. 1; Table 4). This extended incubation period is beyond the scope of previous studies and more than double the incubation time observed in TgM83^{+/-} mice, indicating that the misfolding kinetics for WT α-synuclein are much slower than the A53T mutant. Additionally, MSA inoculations in female TgM20^{+/-} mice induce disease >60 days faster than male mice inoculated with the same patient samples. While the mechanism responsible for these sex differences is currently unclear, it is important that future studies using these models are well-powered to account for sex as a biological variable. It is also quite likely that by investigating the molecular mechanisms that drive these differences in incubation period, disease-modifying factors will be identified that may ultimately become therapeutic targets for patients living with MSA.

Analyzing brain samples collected from the terminal TgM20^{+/-} mice, the presence of pathogenic α-synuclein prions was detected using three different methods: immunostaining for phosphorylated α-synuclein inclusions, α-syn-YFP cell assays to test prion propagation, and Western blots to detect detergent-insoluble phosphorylated α-synuclein. Notably, the distribution of neuropathology in the midbrain and brainstem (FIG. 6A) is consistent with the regional distribution of GCIs in MSA patients, as well as in TgM83^{+/-} mice inoculated with MSA patient samples. The selective ability of the mouse-passaged MSA samples to propagate in some α-syn-YFP cell lines, but not those expressing the E46K mutation or a truncation at residue 95 (FIG. 7), is consistent with the biological strain profile previously reported for MSA prions [36]. These results indicate that MSA prions retain their strain properties following transmission to TgM20^{+/-} mice. Moreover, when comparing data reported here with previous studies transmitting MSA to TgM83^{+/-} mice, the only measurable effect of the A53T mutation is the decrease in the incubation period following inoculation. These findings suggest that even though the A53T mutation is not present in MSA patients, animal and cell models expressing this particular mutation can and should be used to accelerate research on α-synuclein misfolding and spreading in MSA without altering strain properties. The finding that incubation times in the TgM20^{+/-} model are more than double those of the TgM83^{+/-} model highlights the importance of mutant Tg mouse models to the acceleration of MSA research.

Bolstering this interpretation, strain differences in TgM20^{+/-} mice inoculated with WT PFFs were detected compared to those inoculated with MSA patient samples. First, while the MSA patient samples yielded an average incubation period of 281±93 days, the average incubation period for WT PFF-inoculated mice was 210±37 dpi (FIG. 8). It is important to note, though, that this difference in incubation period may be a reflection of differences in titer rather than strain. Second, WT PFF-induced α-synuclein neuropathology spanned additional brain regions from those observed in either TgM20^{+/-} or TgM83^{+/-} mice following MSA inoculation (FIG. 7). As mentioned previously, MSA transmission to TgM20^{+/-} and TgM83^{+/-} mice induces phosphorylated α-synuclein inclusions that are limited to hindbrain areas, with the most predominant inclusions in the midbrain and pons. However, the WT PFF inoculations reported here induced α-synuclein pathology in the hippocampus and piriform cortex & amygdala in addition to the hindbrain regions (FIG. 9A). The density of staining was also substantially greater than that measured following MSA inoculation (comparing percent area measurements in FIG. 7). Finally, brain samples from mice inoculated with the two strains exhibited distinct biological activities when assayed in the α-syn-YFP cells. Unlike the MSA patient samples, the mouse-passaged WT PFFs were able to replicate in α-syn95*A53T-YFP cells (FIG. 10), consistent with previous findings [36]. However, these samples could not replicate in cells expressing the E46K mutation. Several cryo-electron microscopy studies have shown that many WT fibril structures contain a salt bridge between residues E46 and K80, which stabilizes α-synuclein misfolding into a Greek key conformation [11, 17, 18, 23]. As a result, the E46K mutation would disrupt this misfolded structure, preventing the WT fibrils from being able to propagate using E46K α-synuclein as a substrate. While this phenomenon was observed using an in vitro model here, Long et al. obtained similar results using a cell-free cross-seeding approach [20]. Altogether, these differences in incubation period, neuropathological distribution, and α-syn-YFP cell assay infectivity demonstrate that the recombinant WT PFFs and MSA patient samples contain distinct α-synuclein strains. Future studies should investigate the effect of strain titer on these findings.

The studies reported here include pivotal discoveries for the synucleinopathy field for two reasons. First, while non-Tg mice are resistant to MSA transmission [27], this is the first demonstration that MSA prions transmit neurological disease to mice expressing WT human α-synuclein. This is consistent with results from the PrP prion literature showing that, to overcome the human-mouse species barrier, transmission of CJD prions to mice requires the expression of either human PrP or a human-mouse chimeric PrP sequence [14, 32, 33]. These important results add to the known similarities between MSA and CJD prions. Second, these studies identify the third Tg mouse model capable of propagating multiple α-synuclein prion strains, including those found in MSA patient samples. The ability to transmit α-synuclein prion strains to both the TgM20^{+/-} and the TgM83^{+/-} mouse models reveals a unique opportunity to investigate the effect of host genotype on disease pathogenesis. Until now, transmission models relied on either the TgM83^{+/-} or the Tg (SNCA*A53T^{+/+}) Nbm mouse models, both of which harbor the A53T mutation. However, while the A53T mutation is the most common SNCA mutation [28], the vast majority of synucleinopathy patients have WT SNCA genes. Future experiments enabled by the discoveries reported here allow determination of how disease pathogenesis differs in patients with and without familial SNCA mutations.

### F. References for Example 4

The following list of references refers to the citation numbers of this Example:
1 Bernis M E, Babila J T, Breid S, Wüsten K A, Wüllner U, Tamgüney G (2015) Prion-like propagation of human brain-derived alpha-synuclein in transgenic mice expressing human wild-type alpha-synuclein. Acta Neuropathol Commun 3: 75
2 Carlson G A, Kingsbury D T, Goodman P A, Coleman S, Marshall S T, DeArmond S, Westaway D, Prusiner S B (1986) Linkage of prion protein and scrapie incubation time genes. Cell 46:503-511
3 Conway K A, Harper J D, Lansbury P T (1998) Accelerated in vitro fibril formation by a mutant alpha-synuclein linked to early-onset Parkinson disease. Nat Med 4:1318-1320
4 Coon E A, Nelson R M, Sletten D M, Suarez M D, Ahlskog J E, Benarroch E E, Sandroni P, Mandrekar J N, Low P A, Singer W (2019) Sex and gender influence symptom manifestation and survival in multiple system atrophy. Auton Neurosci 219:49-52
5 Dhillon J-S, Trejo-Lopez J A, Riffe C, Levites Y, Sacino A N, Borchelt D R, Yachnis A Y, Giasson B I (2019) Comparative analyses of the in vivo induction and transmission of α-synuclein pathology in transgenic mice by MSA brain lysate and recombinant α-synuclein fibrils. Acta Neuropathol Commun 7:80
6 Emmer K L, Waxman E A, Covy J P, Giasson B I (2011) E46K human alpha-synuclein transgenic mice develop Lewy-like and tau pathology associated with age-dependent, detrimental motor impairment. J Biol Chem 286:35104-35118
7 Eraña H (2019) The Prion 2018 round tables (II): Aβ, tau, α-synuclein. Are they prions, prion-like proteins, or what? Prion 13:41-45
8 Giasson B I, Duda J E, Quinn S M, Zhang B, Trojanowski J Q, Lee V M (2002) Neuronal α-synucleinopathy with severe movement disorder in mice expressing A53T human α-synuclein. Neuron 34:521-533
9 Giasson B I, Uryu K, Trojanowski J Q, Lee V M (1999) Mutant and wild type human alpha-synucleins assemble into elongated filaments with distinct morphologies in vitro. J Biol Chem 274:7619-7622
10 Gilman S, Wenning G K, Low P A, Brooks D J, Mathias C J, Trojanowski J Q, Wood N W, Colosimo C, Dürr A, Fowler C J et al (2008) Second consensus statement on the diagnosis of multiple system atrophy. Neurology 71:670-676
11 Guerrero-Ferreira R, Taylor N M, Mona D, Ringler P, Lauer M E, Riek R, Britschgi M, Stahlberg H (2018) Cryo-EM structure of alpha-synuclein fibrils. eLife 7: pii: e36402
12 Hass E W, Sorrentino Z A, Lloyd G M, McFarland N R, Prokop S, Giasson B I (2021) Robust α-synuclein pathology in select brainstem neuronal populations is a potential instigator of multiple system atrophy. Acta Neuropathol Commun 9: 80
13 Jellinger K A, Lantos P L (2010) Papp-Lantos inclusions and the pathogenesis of multiple system atrophy: An update. Acta Neuropathol 119:657-667
14 Korth C, Kaneko K, Groth D, Heye N, Telling G, Mastrianni J, Parchi P, Gambetti P, Will R, Ironside J et al (2003) Abbreviated incubation times for human prions in mice expressing a chimeric mouse—human prion protein transgene. Proc Natl Acad Sci USA 100:4784-4789
15 Kuo Y M, Li Z, Jiao Y, Gaborit N, Pani A K, Orrison B M, Bruneau B G, Giasson B I, Smeyne R J, Gershon M D et al (2010) Extensive enteric nervous system abnormalities in mice transgenic for artificial chromosomes containing Parkinson disease-associated alpha-synuclein gene mutations precede central nervous system changes. Hum Mol Genet 19:1633-1650
16 Lau A, So R W L, Lau H H C, Sang J C, Ruiz-Riquelme A, Fleck S C, Stuart E, Menon S, Visanji N P, Meisl G et al (2020) α-Synuclein strains target distinct brain regions and cell types. Nat Neurosci 23:21-31
17 Li B, Ge P, Murray K A, Sheth P, Zhang M, Nair G, Sawaya M R, Shin W S, Boyer D R, Ye S et al (2018) Cryo-EM of full-length α-synuclein reveals fibril polymorphs with a common structural kernel. Nat Commun 9: 3609
18 Li Y, Zhao C, Luo F, Liu Z, Gui X, Luo Z, Zhang X, Li D, Liu C, Li X (2018) Amyloid fibril structure of α-synuclein determined by cryo-electron microscopy. Cell Res: DOI: 10.1038/s41422-018-0075-x
19 Lloyd G M, Sorrentino Z A, Quintin S, Gorion K M, Bell B M, Paterno G, Long B, Prokop S, Giasson B I (2022) Unique seeding profiles and prion-like propagation of synucleinopathies are highly dependent on the host in human α-synuclein transgenic mice. Acta Neuropathol:
20 Long H, Zheng W, Liu Y, Sun Y, Zhao K, Liu Z, Xia W, Lv S, Liu Z, Li D et al (2021) Wild-type α-synuclein inherits the structure and exacerbated neuropathology of E46K mutant fibril strain by cross-seeding. Proc Natl Acad Sci USA 118:
21 Luk K C, Covell D J, Kehm V M, Zhang B, Song I Y, Byrne M D, Pitkin R M, Decker S C, Trojanowski J Q, Lee V M-Y (2016) Molecular and biological compatibility with host alpha-synuclein influences fibril pathogenicity. Cell Rep 16: 3373-3387
22 Narhi L, Wood S J, Steavenson S, Jiang Y, Wu G M, Anafi D, Kaufman S A, Martin F, Sitney K, Denis P et al (1999) Both familial Parkinson's disease mutations accelerate alpha-synuclein aggregation. J Biol Chem 274: 9843-9846
23 Ni X, McGlinchey R P, Jiang J, Lee J C (2019) Structural Insights into α-Synuclein Fibril Polymorphism: Effects of Parkinson's Disease-Related C-Terminal Truncations. J Mol Biol 431: 3913-3919
24 Nishie M, Mori F, Yoshimoto M, Takahashi H, Wakabayashi K (2004) A quantitative investigation of neuronal cytoplasmic and intranuclear inclusions in the pontine and inferior olivary nuclei in multiple system atrophy. Neuropathol Appl Neurobiol 30: 546-554
25 Papp M I, Kahn J E, Lantos P L (1989) Glial cytoplasmic inclusions in the CNS of patients with multiple system atrophy (striatonigral degeneration, olivopontocerebellar atrophy and Shy-Drager syndrome). J Neurol Sci 94: 79-100
26 Polymeropoulos M H, Lavedan C, Leroy E, Ide S E, Dehejia A, Dutra A, Pike B, Root H, Rubenstein J, Boyer R et al (1997) Mutation in the α-synuclein gene identified in families with Parkinson's disease. Science 276: 2045-2047
27 Prusiner S B, Woerman A L, Mordes D A, Watts J C, Rampersaud R, Berry D B, Patel S, Oehler A, Lowe J K, Kravitz S N et al (2015) Evidence for α-synuclein prions causing multiple system atrophy in humans with parkinsonism. Proc Natl Acad Sci USA 112: E5308-E5317
28 Rosborough K, Patel N, Kalia L V (2017) α-Synuclein and Parkinsonism: Updates and Future Perspectives. Curr Neurol Neurosci Rep 17: 31
29 Safar J, Wille H, Itri V, Groth D, Serban H, Torchia M, Cohen F E, Prusiner S B (1998) Eight prion strains have PrpSc molecules with different conformations. Nat Med 4: 1157-1165
30 Spillantini M G, Crowther R A, Jakes R, Cairns N J, Lantos P L, Goedert M (1998) Filamentous α-synuclein inclusions link multiple system atrophy with Parkinson's disease and dementia with Lewy bodies. Neurosci Lett 251: 205-208
31 Spillantini M G, Schmidt M L, Lee V M-Y, Trojanowski J Q, Jakes R, Goedert M (1997) α-Synuclein in Lewy bodies. Nature 388: 839-840
32 Telling G C, Scott M, Hsiao K K, Foster D, Yang S-L, Torchia M, Sidle K C L, Collinge J, DeArmond S J, Prusiner S B (1994) Transmission of Creutzfeldt-Jakob disease from humans to transgenic mice expressing chimeric human-mouse prion protein. Proc Natl Acad Sci USA 91: 9936-9940
33 Telling G C, Scott M, Mastrianni J, Gabizon R, Torchia M, Cohen F E, DeArmond S J, Prusiner S B (1995) Prion propagation in mice expressing human and chimeric PrP transgenes implicates the interaction of cellular PrP with another protein. Cell 83: 79-90
34 Wakabayashi K, Miki Y, Tanji K, Mori F (2022) Neuropathology of Multiple System Atrophy, a Glioneuronal Degenerative Disease. Cerebellum:
35 Watts J C, Giles K, Oehler A, Middleton L, Dexter D T, Gentleman S M, DeArmond S J, Prusiner S B (2013) Transmission of multiple system atrophy prions to transgenic mice. Proc Natl Acad Sci USA 110: 19555-19560
36 Woerman A L, Kazmi S A, Patel S, Aoyagi A, Oehler A, Widjaja K, Mordes D A, Olson S H, Prusiner S B (2018) Familial Parkinson's point mutation abolishes multiple system atrophy prion replication. Proc Natl Acad Sci USA 115: 409-414
37 Woerman A L, Kazmi S A, Patel S, Freyman Y, Oehler A, Aoyagi A, Mordes D A, Halliday G M, Middleton L T, Gentleman S M et al (2018) MSA prions exhibit remarkable stability and resistance to inactivation. Acta Neuropathol 135: 49-63
38 Woerman A L, Oehler A, Kazmi S A, Lee J, Halliday G M, Middleton L T, Gentleman S M, Mordes D A, Spina S, Grinberg L T et al (2019) Multiple system atrophy prions retain strain specificity after serial propagation in two different Tg (SNCA*A53T) mouse lines. Acta Neuropathol 137: 437-454
39 Woerman A L, Patel S, Kazmi S A, Oehler A, Lee J, Mordes D A, Olson S H, Prusiner S B (2020) Kinetics of α-synuclein prions preceding neuropathological inclusions in multiple system atrophy. PLOS Pathog 16: e1008222
40 Woerman A L, Stöhr J, Aoyagi A, Rampersaud R, Krejciova Z, Watts J C, Ohyama T, Patel S, Widjaja K, Oehler A et al (2015) Propagation of prions causing synucleinopathies in cultured cells. Proc Natl Acad Sci USA 112: E4949-E4958

### Example 4. Example 5. The E46K Mutation Modulates α-Synuclein Prion Replication in Transgenic Mice

In MSA, the α-synuclein protein misfolds into a self-templating prion conformation that spreads throughout the brain, leading to progressive neurodegeneration. While the E46K mutation in α-synuclein causes familial Parkinson's disease (PD), this mutation has recently been shown to block in vitro propagation of MSA prions. Recent studies by others indicate that α-synuclein adopts a misfolded conformation in MSA in which a Greek key motif is stabilized by an intramolecular salt bridge between residues E46 and K80. Hypothesizing that the E46K mutation impedes salt bridge formation and, therefore, exerts a selective pressure that can modulate α-synuclein strain propagation, three distinct α-synuclein prion strains were tested for propogation in TgM47^{+/-} mice, which express human α-synuclein with the E46K mutation. Following intracranial injection of these strains, TgM47^{+/-} mice were resistant to MSA prion transmission, whereas recombinant E46K preformed fibrils (PFFs) transmitted neurological disease to mice and induced the formation of phosphorylated α-synuclein neuropathology. In contrast, heterotypic seeding following wild-type (WT) PFF-inoculation resulted in preclinical α-synuclein prion propagation. Moreover, when TgM20^{+/-} mice, which express WT human α-synuclein, with E46K PFFs, were inoculated, delayed transmission kinetics with an incomplete attack rate were seen. These findings suggest that the E46K mutation constrains the number of α-synuclein prion conformations that can propagate in TgM47^{+/-} mice, expanding the understanding of the selective pressures that impact α-synuclein prion replication.

The underlying cause of disease in a group of movement disorders called syncleinopathies is the misfolding of the protein α-synuclein into a shape that self-templates to spread disease. The type of synucleinopathy that a patient develops depends on the shape α-synuclein adopts during misfolding. For example, α-synuclein misfolds into one shape in patients with PD and into another in patients with MSA. These distinct forms are referred to as strains. Understanding α-synuclein strain biology, and the factors that impact strain formation and spread, is imperative for developing successful diagnostics and therapeutics. In this study, the effect of the E46K mutation in α-synuclein, which causes PD, was investigated on α-synuclein strain biology to determine how the patient's genetic background can impact disease. Results showed that this particular mutation modulates α-synuclein spread: while mice expressing E46K α-synuclein were resistant to MSA prion replication, synthetic E46K fibrils induced neurological disease in the mice. In contrast, heterotypic seeding of wild-type synthetic fibrils yielded inefficient propagation that could not induce neurological signs in the mice. These results indicate that patient genotype can greatly impact α-synuclein strain formation and, therefore, clinical presentation in synucleinopathy patients.

Synucleinopathies are a group of neurodegenerative diseases caused by the misfolding and self-templating of the α-synuclein protein. In one group of diseases, α-synuclein misfolds and aggregates into neuronal Lewy bodies and Lewy neurites [36]. This group, collectively referred to as Lewy body diseases (LBDs), includes PD, PD with dementia (PDD), and dementia with Lewy bodies (DLB). In contrast, in patients with MSA, α-synuclein misfolds into glial and neuronal cytoplasmic inclusions. While MSA and LBD patients can present with overlapping motor deficits, MSA patients are typically diagnosed earlier in life (50-60 years old rather than >70 years old) and experience a more rapid disease progression compared to LBD patients [10-12]. However, while there are eleven known familial LBD mutations in the gene encoding α-synuclein, SNCA, no mutations have been identified in MSA patients [1, 4, 7, 20-23, 26, 29, 30, 49, 50].

To investigate the role of α-synuclein misfolding and self-templating in synucleinopathies, TgM83^{+/-} mice, which express human SNCA*A53T [9], were previously inoculated with homogenates prepared from brain samples of deceased MSA patients [31, 41]. Unexpectedly, while the MSA patient samples induced neurological disease ˜120 days postinoculation (dpi), none of the PD, DLB, or PDD patient samples tested transmitted disease to the mice. More recently, Thomzig et al. found that brain tissue from two deceased PD patients induced subclinical neuropathological changes in the brain, absent the onset of neurological signs [38]. In parallel, a HEK293T cell assay expressing α-syn140*A53T-YFP was used to propagate α-synuclein prions isolated from MSA patient samples but not those from the PD or DLB patients [47]. These findings support a hypothesis that MSA is caused by α-synuclein misfolding into a distinct conformation, or strain, compared to the α-synuclein fibrils present in LBD patients.

Being unable to transmit DLB prions in cultured cells or Tg mice, a panel of α-syn-YFP cell lines were built expressing SNCA mutations and were tested for the ability of MSA prions to replicate using mutant protein [43]. MSA prions can propagate using wild-type (WT), A30P, and A53T α-synuclein, but the presence of the PD-causing E46K mutation prevented MSA prion replication in vitro. That a single point mutation in α-synuclein interferes with prion propagation is not surprising: the Canidae family was found to be resistant to PrP prion disease due to the presence of a single residue polymorphism at position 163 in the PrP protein [37]. Additionally, the Q171R mutation in sheep and the E219K mutation in humans both confer resistance to prion disease [13, 35, 42].

Several lines of research have focused on understanding why the E46K mutation exhibits a unique effect on α-synuclein pathogenesis. Testing the ability of WT α-synuclein preformed fibrils (PFFs) to induce neuropathological inclusions in TgM47 mice, which express SNCA*E46K [6], Sacino et al. reported inefficient transmission with limited inclusions around the injection site at 4 months postinoculation (mpi) [32]. Notably, similar studies in the TgM83 animals resulted in widespread neuronal inclusions throughout the brain. In 2016, Tuttle et al. used solid-state nuclear magnetic resonance to resolve the structure of WT α-synuclein fibrils and reported that a salt bridge between residues E46 and K80 stabilizes a Greek key motif within the fibril structure [39]. Subsequent cryo-electron microscopy (cryo-EM) studies have identified the E46/K80 salt bridge in WT recombinant fibrils as well as fibrils isolated from MSA patient samples [16, 24, 25, 28, 34]. Consistent with the hypothesis that the E46K mutation disrupts this interaction, the lack of the salt bridge in two cryo-EM structures resolved using E46K fibrils results in conformations that vary substantially from those reported for WT fibrils [2, 51]. Moreover, Long et al. showed that WT fibrils containing the E46/K80 salt bridge are unable to propagate E46K α-synuclein prions, but E46K fibrils can template using WT protein as a substrate [27]. It is important to note that the cryo-EM structures reported using either WT or mutant PFFs vary substantially from the conformations resolved using MSA patient samples (reviewed in [18]). These conformations are also structurally distinct from the recently resolved α-synuclein fibrils isolated from LBD patient samples, which show that K80 forms a salt bridge with E35, rather than E46, in LBD patients [48]. Altogether, a growing body of literature suggests that the E46K mutation is likely to exert selective pressures on α-synuclein prion propagation different from the effects of other familial SNCA mutations.

To understand the effect of the E46K mutation on α-synuclein prion strain propagation, the ability of three different α-synuclein prion sources to replicate were examined in TgM47^{+/-} mice. Consistent with cell assay data, the E46K mutation inhibited MSA transmission to the mice over a 475-day incubation period. Conversely, inoculations using E46K PFFs transmitted disease to the animals; however, a delayed incubation period suggests that the kinetics of E46K α-synuclein self-templating are slowed compared to the well-studied A53T mutation. Distinct from both of these results, heterotypic seeding in which TgM47^{+/-} mice were inoculated with WT PFFs resulted in preclinical α-synuclein prion propagation. Finally, inoculation of E46K PFFs was tested in the TgM20^{+/-} mouse model, which expresses WT human α-synuclein [9], and disease transmission was observed with an incomplete attack rate. These findings not only indicate that prion propagation using E46K protein as substrate is limited to a subset of α-synuclein conformations, but they also contribute to an understanding of the selective pressures that impact α-synuclein prion replication.

### A. The PD-Causing E46K Mutation in α-Synuclein Inhibits MSA Prion Transmission

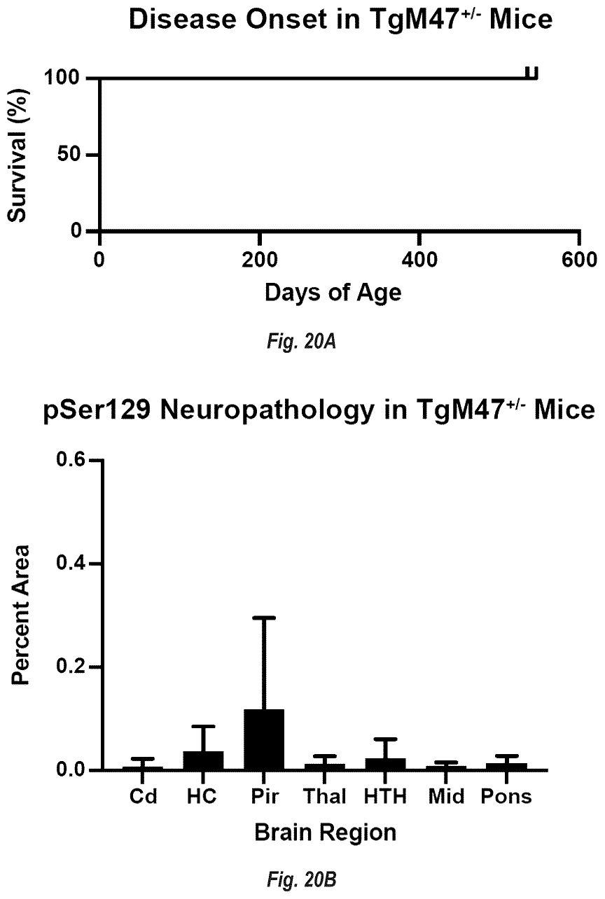

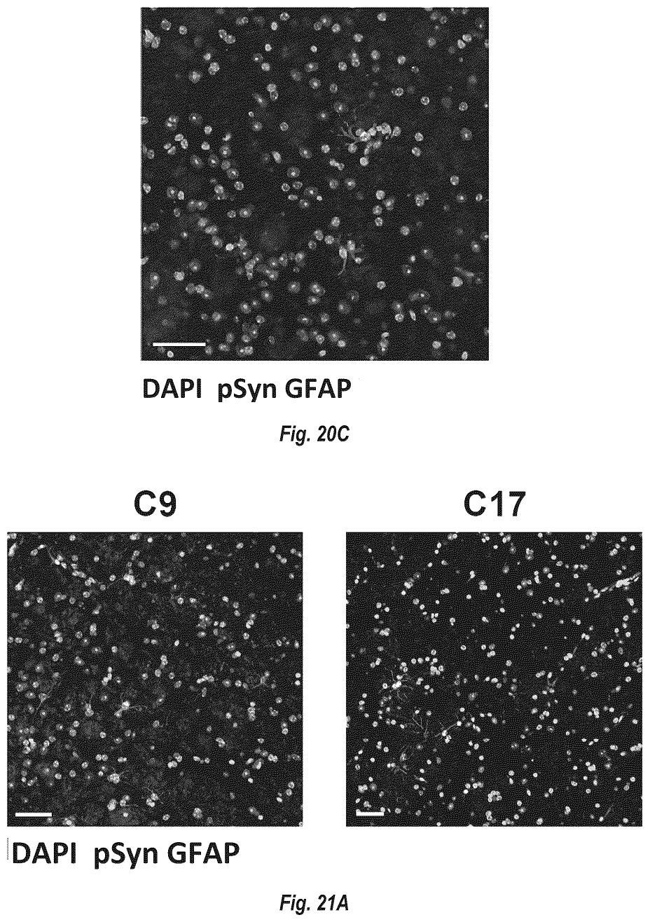

Previously, a panel of α-syn-YFP cell lines was used to demonstrate that the E46K mutation, which causes PD and DLB [50], interferes with in vitro replication of MSA prions [43]. To determine if these findings in cultured cells are predictive of in vivo α-synuclein strain biology, it was investigated whether MSA prions can transmit disease to transgenic (Tg) mice expressing the same mutation. The TgM47 mouse model uses the Prnp promoter to drive expression of human α-synuclein with the E46K mutation [6]. Homozygous TgM47+/+ mice develop spontaneous neurological disease with an onset between 16 and 29 months of age, which is accompanied by accumulation of phosphorylated α-synuclein aggregates in the hindbrain and spinal cord [6]. Alternatively, hemizygous TgM47^{+/-} mice did not develop neurological signs by 545 days of age (˜18 months: FIG. 20A) and lack pathological α-synuclein accumulation in the brain, as determined using both a panel of α-syn-YFP cells (Table 5) and immunostaining of fixed tissue for phosphorylated α-synuclein (FIGS. 20B and C). Given this absence of spontaneous disease, hemizygous mice were chosen for the experiments in this study.

TABLE 5
Infectivity of aged TgM47^{+/-} samples in cultured cells.
Mean cell infection ± SD (Fluorescence/Cell × 10³ A.U.)

| α-syn140- | α-syn140*E46K- | α-syn140*A53T- | α-syn140*E46K, | α-syn95*A53T- |
|---|---|---|---|---|
| YFP | YFP | YFP | A53T-YFP | YFP |
| 1.0 ± 0.5 | 0.8 ± 0.2 | 1.7 ± 0.8 | 0.8 ± 0.4 | 3.4 ± 0.1 |

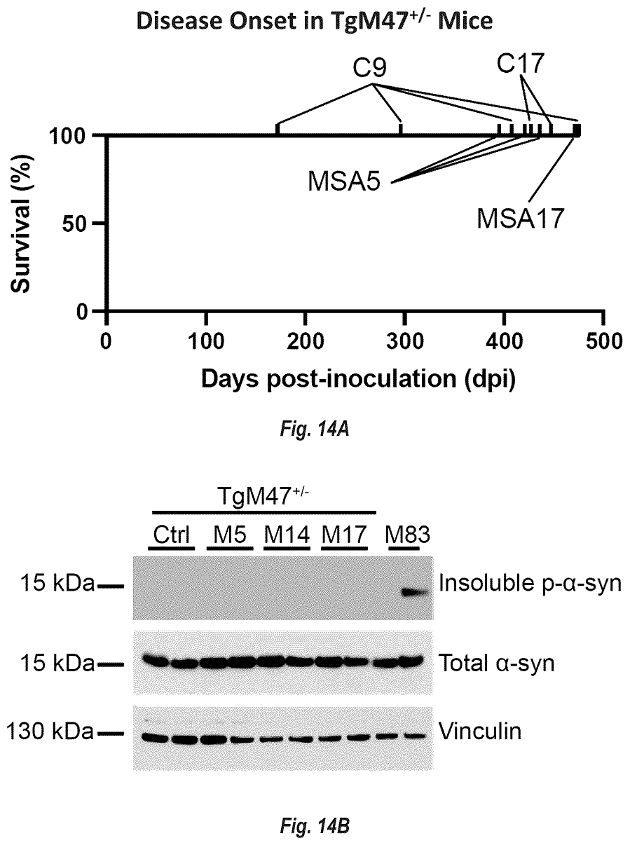

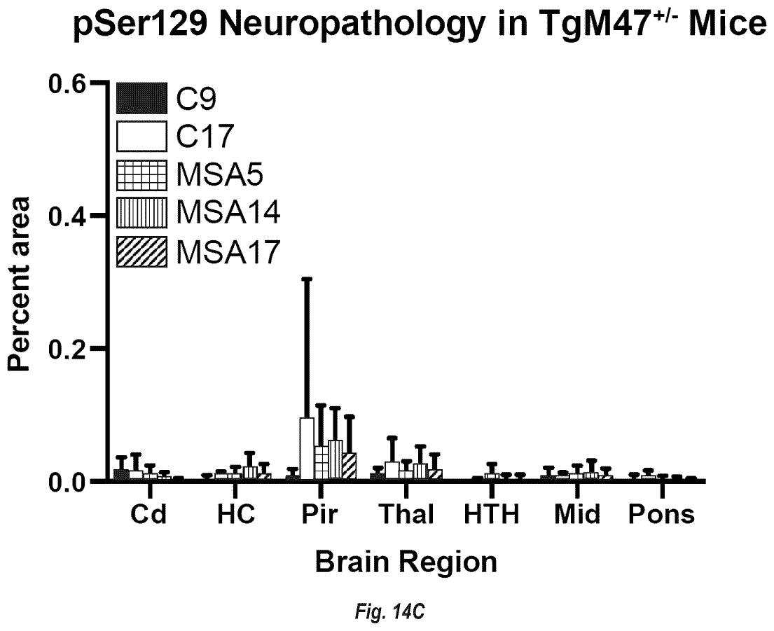

To determine if MSA prions replicate in Tg mice expressing E46K α-synuclein in vivo, eight-week-old TgM47^{+/-} mice were inoculated intracranially (i.e.) with 30 μL 1% brain homogenate prepared from either two control patient samples (C9 and C17) or three MSA patient samples (MSA5, MSA14, and MSA17; Table 6) and monitored the mice for the onset of neurological signs (FIGS. 14A-14C).

TABLE 6
Infectivity of TgM47+/−-passaged MSA prions in cultured cells

| | Mean cell infection ± SD | | | | |
|---|---|---|---|---|---|
| | (Fluorescence/Cell × 10³ A.U.) | | | | |
| | a-syn140- | a-syn140*E46K- | a-syn140*A53T- | a-syn140*E46K, | a-syn95*A53T- |
| Sample | YFP | YFP | YFP | A53T-YFP | YFP |
| C9 | 1.0 ± 0.3 | 1.2 ± 1.0 | 1.7 ± 1.0 | 0.3 ± 0.1 | 2.0 ± 2.6 |
| C17 | 0.8 ± 0.2 | 0.8 ± 0.2 | 1.0 ± 0.2 | 0.5 ± 0.1 | 1.3 ± 0.3 |
| MSA5 | 0.9 ± 0.3 | 0.8 ± 0.2 | 1.4 ± 0.2 | 0.4 ± 0.1 | 1.2 ± 0.1 |
| MSA14 | 0.8 ± 0.1 | 0.9 ± 0.3 | 1.4 ± 0.3 | 0.4 ± 0.1 | 1.4 ± 0.1 |
| MSA17 | 1.0 ± 0.1 | 0.8 ± 0.2 | 1.3 ± 0.2 | 0.4 ± 0.1 | 1.7 ± 0.4 |

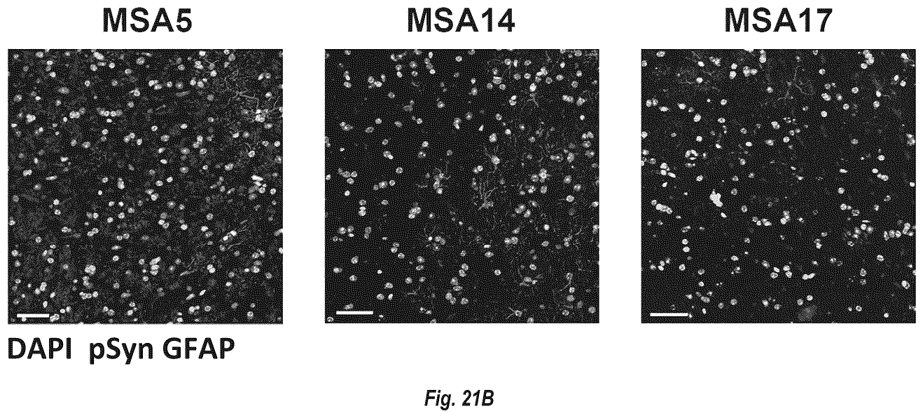

Consistent with in vitro findings, all animals remained healthy at 475 dpi, regardless of inocula (FIG. 14A; P>0.05). To determine if the MSA-inoculated mice were preclinical at the time of collection, aliquots of frozen half-brains were tested in a panel of α-syn-YFP cell lines. None of the samples were able to infect the cell lines tested (Table 6; P>0.05). Sarkosyl-insoluble α-synuclein was not detected in the mouse samples via Western blot, though control- and MSA-inoculated TgM47^{+/-} mice expressed similar levels of total α-synuclein (FIG. 14B). Finally, fixed half-brains from the same mice were tested for the presence of phosphorylated α-synuclein inclusions, and neither the control nor the MSA patient samples induced α-synuclein pathology in the TgM47^{+/-} mice (FIG. 14C and FIGS. 21A and B; P>0.05). Together, these findings indicate that MSA prions are unable to replicate in vivo using E46K α-synuclein as substrate.

### B. E46K PFFs Replicate Slowly in TgM47^{+/-} Mice

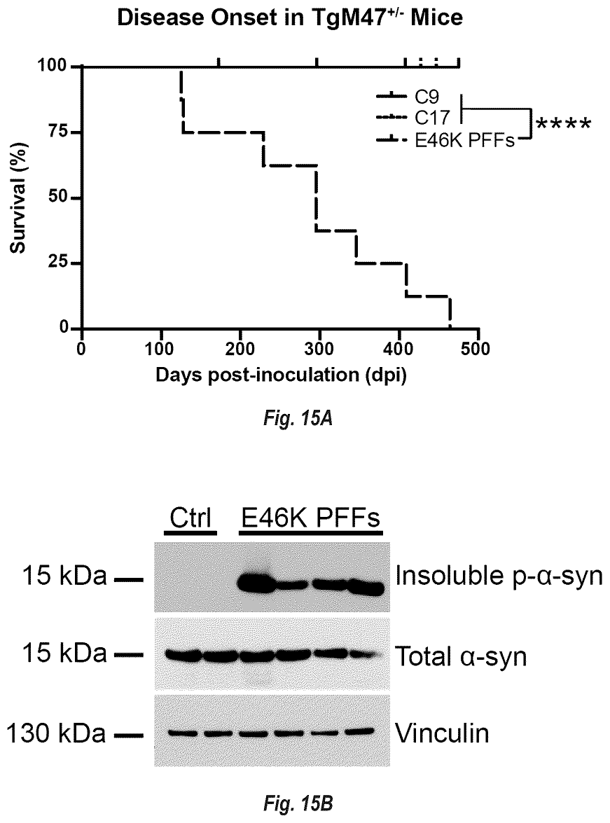

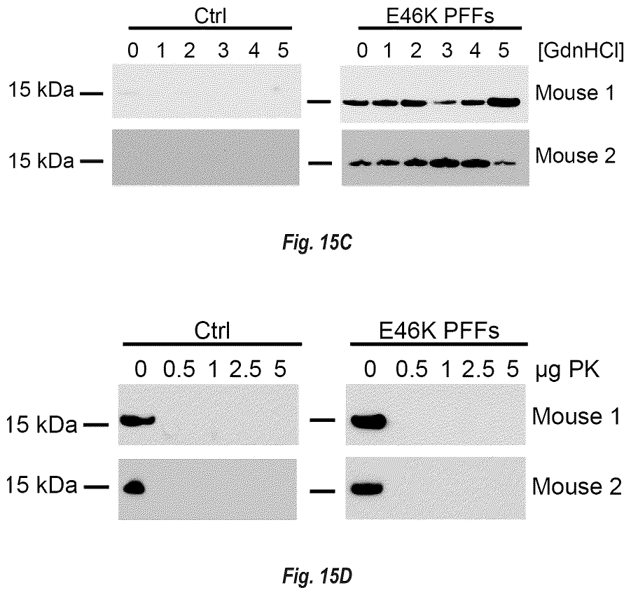

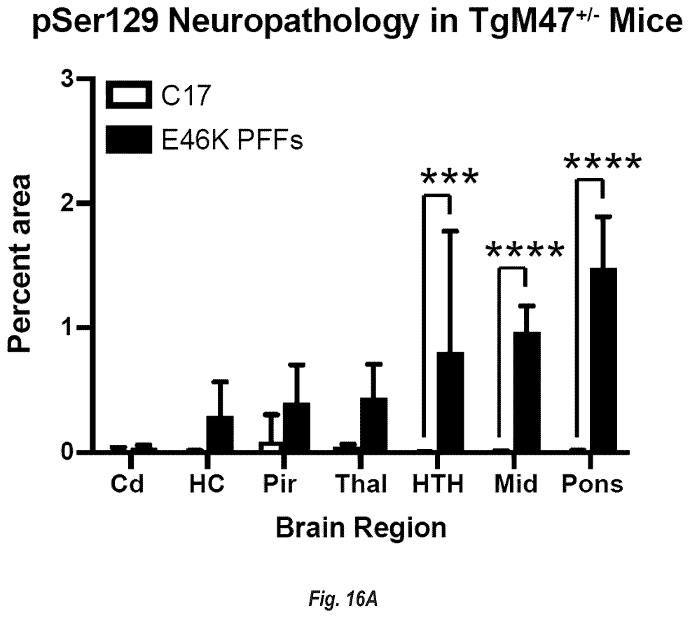

To confirm that TgM47^{+/-} mice can develop synucleinopathy following i.e. injection with α-synuclein prions, eight-week-old mice were inoculated with recombinant E46K or WT α-synuclein PFFs. α-Synuclein monomers containing an N-terminal His tag (Sigma Aldrich) were fibrillized in DPBS at 37° C. with shaking for 1 week. To first test homotypic fibril transmission, TgM47^{+/-} mice were inoculated with either 30 μL of 1% (wt/vol) brain homogenate from two control patient samples or 30 μg of E46K PFFs (FIGS. 15A-D and 16A-B; Table 7). The mice were then evaluated for the onset of neurological signs twice each week. Unlike the control-inoculated animals, TgM47^{+/-} mice inoculated with the E46K PFFs developed neurological signs 286±122 dpi (FIG. 15A; P<0.0001). Notably, substantial variability in disease onset in the mice: the first animal developed clinical signs at 125 dpi while the last mouse did not show signs until 464 dpi. Compared to transmission studies in the TgM83^{+/-} mice, where disease onset occurs within 120±26 dpi on average [31, 44, 47], the observed incubation period in the TgM47^{+/-} mice is both delayed and extended. One possible explanation for these findings is that E46K PFFs propagate with slower kinetics than A53T α-synuclein, as has been shown previously [14]. Supporting this hypothesis, E46K PFF-inoculated mouse samples were only able to propagate in the E46K,A53T cells when tested in the α-syn-YFP cell assay (Table 7; P<0.0001). Therefore, it is likely that, in the presence of both the E46K and A53T mutations, the α-syn-YFP cells can support homotypic templating while also accelerating misfolding kinetics, enabling detection of a slower propagating E46K prion strain.

TABLE 7
Infectivity of TgM47+/−-passaged E46K PFFs in cultured cells.

| | Mean cell infection ± SD | | | | |
|---|---|---|---|---|---|
| | (Fluorescence/Cell × 103 A.U.) | | | | |
| | a-syn140- | a-syn140*E46K- | a-syn140*A53T- | a-syn140*E46K, | a-syn95*A53T- |
| Sample | YFP | YFP | YFP | A53T-YFP | YFP |
| C9 | 1.0 ± 0.3 | 1.2 ± 1.0 | 1.7 ± 1.0 | 0.3 ± 0.1 | 2.0 ± 2.6 |
| C17 | 0.8 ± 0.2 | 0.8 ± 0.2 | 1.0 ± 0.2 | 0.5 ± 0.1 | 1.3 ± 0.3 |
| E46K | 0.8 ± 0.1 | 0.7 ± 0.1 | 1.3 ± 0.4 | 4.8 ± 0.1*** | 1.8 ± 0.4 |
| PFFs | | | | | |

***= P < 0.001 compared to control-patient samples C9 and C17.

Biochemical analysis of sarkosyl-insoluble α-synuclein aggregates from mice inoculated with E46K PFFs showed that the pathogenic protein in the clinical animals was phosphorylated at residue S129 (FIG. 15B). Consistent levels of total α-synuclein expression were seen across inoculated animals, eliminating the possibility that differences in substrate expression contributed to the varied incubation times seen in the mice. Intriguingly, GdnHCl denaturation did not degrade α-synuclein aggregates, detected using the total α-synuclein antibody MJFR1, in the E46K PFF-inoculated brain homogenates (FIG. 15C), but any amount of PK digestion did (FIG. 15D). These findings indicate the aggregates are resistant to chaotropic agents while remaining susceptible to proteases: however, it is possible that different results would be obtained using a milder protease, such as thermolysin, or an antibody for a different region of the protein.

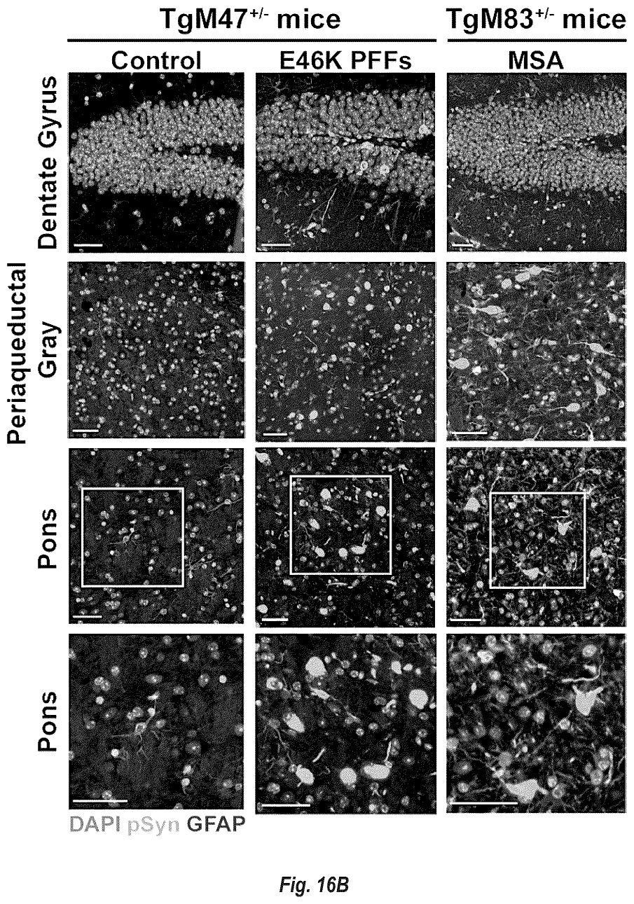

Lastly, fixed half-brains were used to assess the ability of E46K PFFs to induce phosphorylated α-synuclein pathology in the TgM47^{+/-} mice (FIGS. 16A-16B). While α-synuclein inclusions in the hippocampus, piriform cortex and amygdala, and thalamus were observed that were not statistically different from control-inoculated animals (P>0.05), the neuropathology present in the hypothalamus, midbrain, and pons was significantly increased compared to the control-injected animals (FIG. 16A: P<0.001). Notably, the perinuclear inclusions observed in the E46K PFF-inoculated TgM47^{+/-} mice differ from the pathology observed in MSA-inoculated TgM83^{+/-} mice, which is characterized by the presence of phosphorylated α-synuclein throughout the neuronal cell body and neurites in the hindbrain (FIG. 16B). These data demonstrate that E46K PFFs propagate in TgM47^{+/-} mice with delayed kinetics, inducing neurological disease and the formation of mature, phosphorylated inclusions in the brain.

### C. WT PFFs Propagate Inefficiently in TgM47^{+/-} Mice

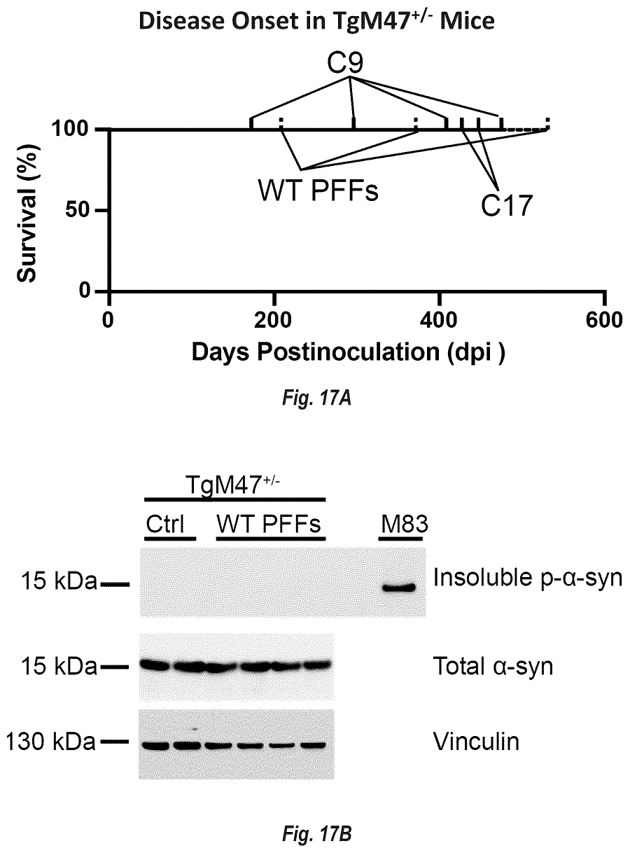

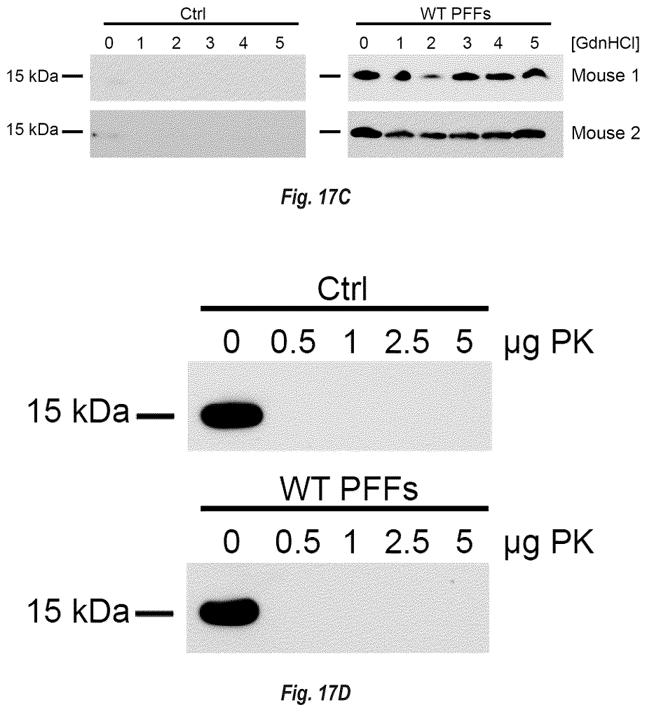

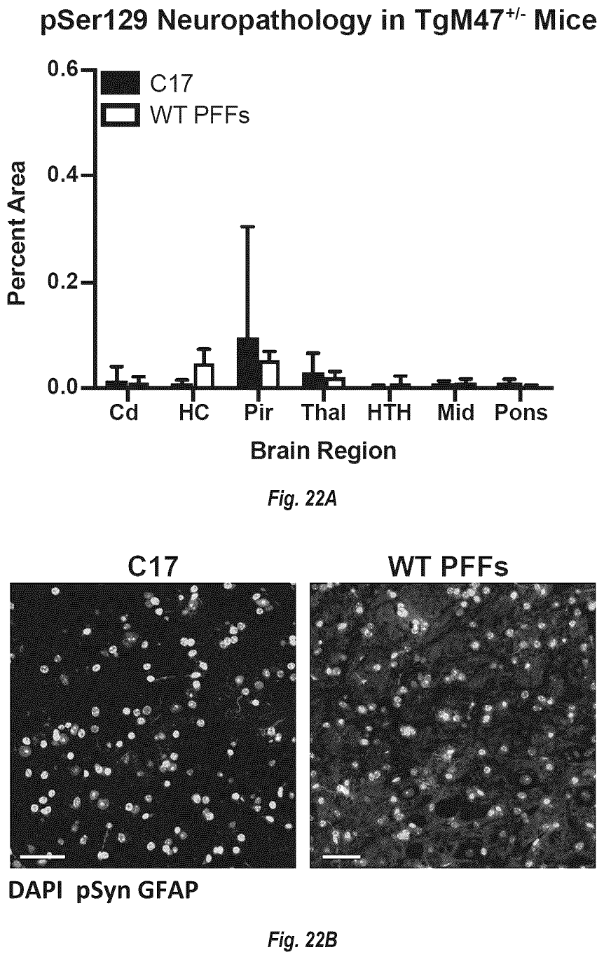

Intracerebral injection of recombinant WT fibrils (containing α-synuclein residues 21-140) into the hippocampus of TgM47 mice was previously shown to induce minimal pathology 4 mpi [32]. More recently, studies by Long et al. reported that WT fibrils could not nucleate fibril elongation using E46K α-synuclein as substrate [27]. To determine if the E46K mutation exerts a selective pressure on WT PFF propagation in vitro, eight-week-old TgM47^{+/-} mice were inoculated with 30 μg of recombinant WT PFFs (FIGS. 17A-17D, Table 8, and FIGS. 22A and B). By 531 dpi, none of the inoculated animals developed neurological signs (FIG. 17A; P>0.05); however, brain homogenates from WT PFF-inoculated mice contained α-synuclein prions that propagated in the WT and A53T cell lines (Table 8; P<0.0001). In contrast, these samples could not replicate in the E46K, E46K,A53T, or truncated α-syn*A53T (1-95) cell lines (P>0.05).

TABLE 8
Infectivity of TgM47+/−-passaged WT PFFs in cultured cells

| | Mean cell infection ± SD | | | | |
|---|---|---|---|---|---|
| | (Fluorescence/Cell × 10³ A.U.) | | | | |
| | a-syn140- | a-syn140*E46K- | a-syn140*A53T- | a-syn140*E46K, | a-syn95*A53T- |
| Sample | YFP | YFP | YFP | A53T-YFP | YFP |
| C9 | 1.0 ± 0.3 | 1.2 ± 1.0 | 1.7 ± 1.0 | 0.3 ± 0.1 | 2.0 ± 2.6 |
| C17 | 0.8 ± 0.2 | 0.8 ± 0.2 | 1.0 ± 0.2 | 0.5 ± 0.1 | 1.3 ± 0.3 |
| WT | 9.7 ± 5.1**** | 0.1 ± 0.0 | 21 ± 6.3**** | 1.5 ± 0.8 | 4.4 ± 2.8 |
| PFFs | | | | | |

****= P < 0.0001 compared to control-patient samples C9 and C17.

Previously, recombinant PFFs were proposed to likely exist as a mixture of conformations, some of which propagate better than others under varying selective pressures [19, 43]. One result of this variability is that only a fraction of the total conformations present can replicate using E46K α-synuclein as substrate. Consequently, when a mixture of WT PFFs is passaged in TgM47^{+/-} mice, as tested here, the animal genotype exerts a selective pressure on the inoculum, selecting for replication-competent α-synuclein species as the strain replicates in a new host. This essentially creates a low titer inoculation where the α-synuclein prions replicate, but the mice remain in a preclinical stage at the time of collection. Supporting this hypothesis, a change in strain biology following passaging of WT PFFs in the TgM47^{+/-} mice was observed. Prior to inoculation, the WT fibrils propagated in all of the α-syn-YFP cell lines tested [43], but after transmission, replication was only detected in two cell lines (Table 8). Subsequent passaging studies using homogenates from these mice are needed to determine if strain adaptation has occurred.

Cell-based models for protein misfolding are more sensitive to the presence of pathogenic protein in a sample than other commonly used detection methods [45, 46]. To assess the stage of preclinical WT PFF replication in the TgM47^{+/-} mice, biochemistry and immunohistochemistry were used to characterize the pathogenic α-synuclein present in the brains of the mice. Western blots were first used to probe for the presence of sarkosyl-insoluble phosphorylated α-synuclein isolated from fresh-frozen brain homogenates (FIG. 17B). Neither control-inoculated nor WT PFF-inoculated mice contained sarkosyl-insoluble α-synuclein. These results are consistent with the idea that some posttranslational modifications, including phosphorylation, occur after self-templating rather than before [8]. GdnHCl denaturation was then used to detect the presence of GdnHCl-resistant α-synuclein aggregates using an antibody for total α-synuclein (MJFR1; FIG. 17C). While no GdnHCl-resistant α-synuclein was detected in the brains of control-inoculated mice, the brains of WT PFF-inoculated mice contained GdnHCl-resistant protein aggregates. Notably, similar to data collected from mice inoculated with E46K PFFs, the amount of total α-synuclein remaining in the samples following denaturation with 5 M GdnHCl was unchanged from the amount of total α-synuclein following incubation without GdnHCl. These findings indicate that even though α-synuclein phosphorylation was not detected at residue S129, the insoluble aggregates that are present are highly resistant to chaotropic agents. In contrast, α-synuclein resistant to PK digestion was not detected in control-inoculated or WT PFF-inoculated animals (FIG. 17D). Though, as previously mentioned, these results may be impacted by protease and antibody epitope. Finally, immunostained fixed-tissue sections were analyzed from the same mice for the presence of α-synuclein neuropathology (FIGS. 22A and B). Consistent with biochemistry experiments, the WT PFFs failed to induce the formation of phosphorylated α-synuclein inclusions. These findings suggest that the WT PFF inoculum contained a low titer of replication-competent conformations of α-synuclein prions.

An important caveat to consider regarding these findings is the possible persistence of inoculum in the mouse brain. However, the altered biological activity of the brain homogenate in the α-syn-YFP cell lines is consistent with strain selection rather than persistent inoculum. Moreover, in the unlikely event that all 30 μg of WT PFFs remained in the brain by 531 dpi, the maximum concentration of WT PFFs incubated with the cells would be ˜0.0008 μg/μL. Titration curves previously tested using WT PFFs on cell lines found the detectable limit is ˜0.25 μg/μL [43], indicating that infection in the α-syn-YFP cells cannot be due to the presence of persistent inoculum in the absence of replication.

### D. E46K PFFs Replication Kinetics are Slowed when Using WT α-Synuclein as Substrate

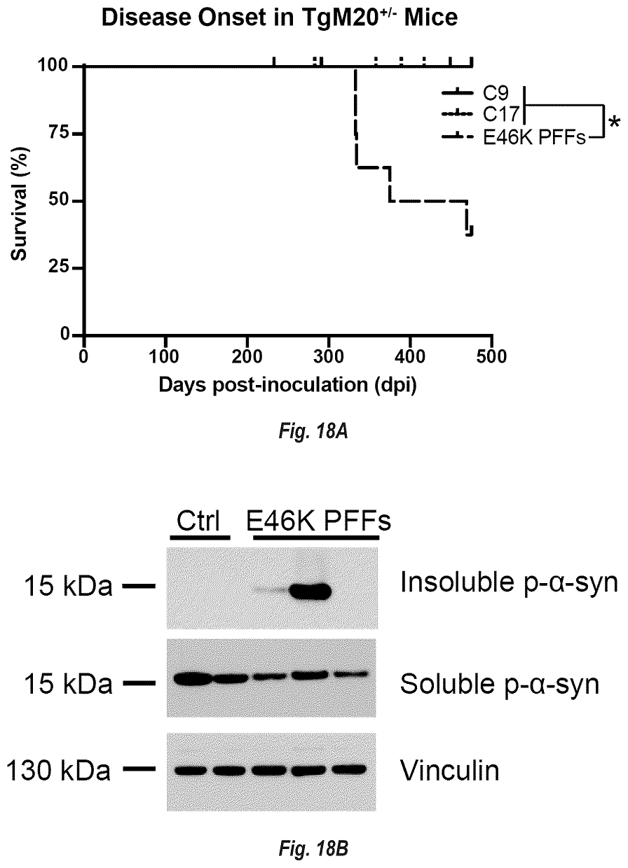

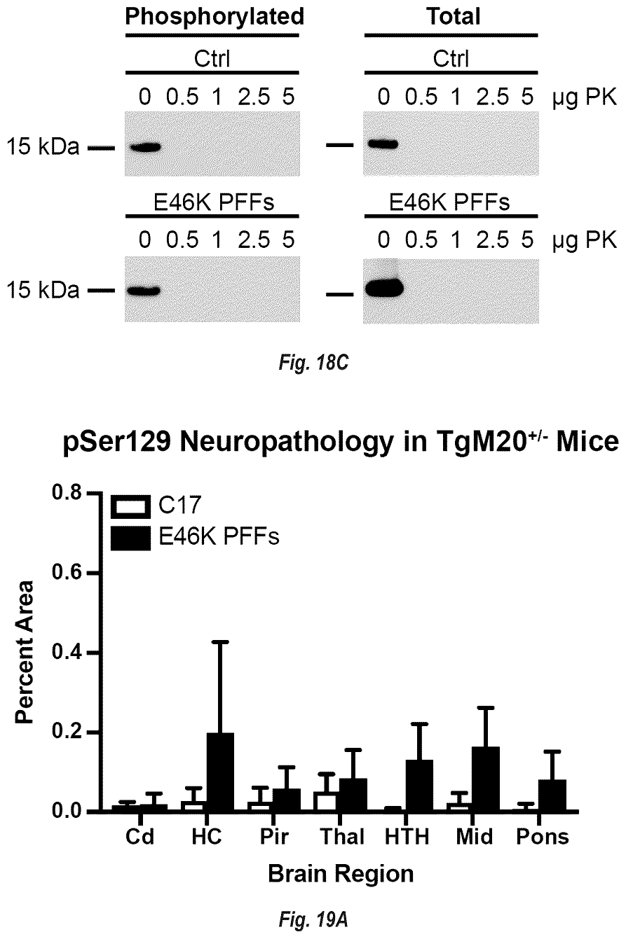

Considering that WT PFFs inefficiently replicate when forced to use E46K α-synuclein as substrate, the question of how propagation of E46K PFFs is impacted during heterotypic replication was studied. In these experiments, the TgM20^{+/-} mouse model that uses the Prnp promoter was utilized to express WT human α-synuclein [9], making this the best model available to test this effect. Both WT PFFs and MSA patient samples propagate with high fidelity in TgM20^{+/-} mice, inducing neurological disease over an extended incubation period compared to transmission studies in the TgM83^{+/-} mouse model [17]. Here, eight-week-old TgM20^{+/-} mice inoculated with 30 μL of 1% brain homogenate from control patient samples or 30 μg E46K PFFs were assessed biweekly for the onset of neurological signs. While none of the control-inoculated mice developed disease, five of the eight mice inoculated with E46K PFFs developed disease before the experiment concluded at 475 dpi (FIG. 18A; P<0.05). Unexpectedly, α-synuclein prions isolated from mice inoculated with E46K PFFs propagated in the WT, A53T, A30P,A53T, E46K,A53T, and truncated α-syn*A53T (1-95) cell lines (P<0.0001) but not in the E46K cells (Table 9; P>0.05). Moreover, two of the healthy mice euthanized at 475 dpi were positive for α-synuclein prions, indicating preclinical propagation in the animals. These data suggest that the kinetics of E46K PFF propagation are slowed when forced to use WT α-synuclein as substrate.

TABLE 9
Infectivity of TgM20^{+/-}-passaged E46K PFFs in cultured cells

| | Mean cell infection ± SD^{a} | | | | |
|---|---|---|---|---|---|
| | (Fluorescence/Cell × 10³ A.U.) | | | | |
| | α-syn140- | α-syn140*E46K- | α-syn140*A53T- | α-syn140*E46K, | α-syn95*A53T- |
| Sample | YFP | YFP | YFP | A53T-YFP | YFP |
| C9 | 1.0 ± 0.3 | 1.0 ± 0.2 | 2.4 ± 1.1 | 0.8 ± 0.9 | 2.1 ± 1.1 |
| C17 | 1.0 ± 0.5 | 0.9 ± 0.2 | 2.2 ± 1.3 | 0.4 ± 0.2 | 1.7 ± 1.2 |
| E46K | 13 ± 6.7**** | 0.9 ± 0.9 | 18 ± 7.3**** | 9.6 ± 4.5**** | 8.4 ± 6.6**** |
| PFFs | | | | | |

^{a}Cell infection data from mice inoculated with samples C9 and C17 reported in Holec, et al., 2022 [17].
****= P < 0.0001 compared to control-patient samples C9 and C17.

Biochemical assays were next used to characterize the pathogenic α-synuclein in the resulting brain homogenates (FIGS. 18B and 18C). Sarkosyl-extracted protein pellets from control-inoculated mice did not contain phosphorylated α-synuclein, but some of the pellets from E46K PFF-inoculated mice did (FIG. 18B). PK was used to determine if the E46K prions were resistant to digestion after passaging in TgM20^{+/-} mice. First, blots were probed using the EP1536Y primary antibody to detect α-synuclein phosphorylated at residue S129, and bands were only present in undigested samples (FIG. 18C, left). Moreover, blots probed using the MJFR1 primary antibody for total α-synuclein also failed to detect PK-resistant α-synuclein (FIG. 18C, right), indicating that the E46K strain loses PK resistance after passaging in Tg mice. It is possible, however, that α-synuclein fibrils in these mice are resistant to milder proteases or are detectable using antibodies with epitopes directed to other regions of the protein.

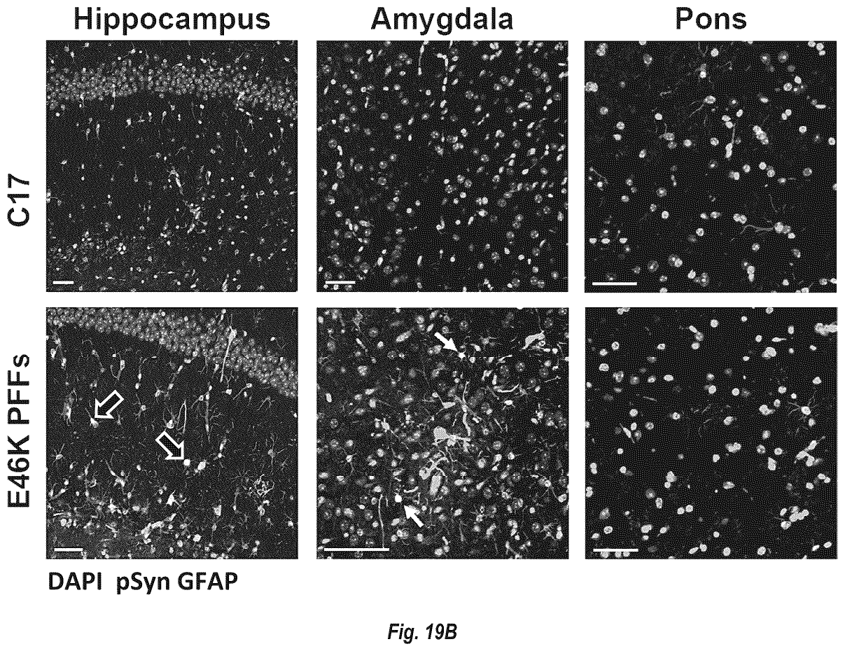

Finally, immunostained fixed sections were analyzed for the presence of α-synuclein neuropathology (FIGS. 19A-19B). The E46K PFFs failed to induce any significant neuropathological inclusions in the TgM20^{+/-} mice (FIG. 19A). However, some phosphorylated α-synuclein immunostaining was observed in the hippocampus and amygdala in the four brains available for analysis from the five clinical mice (FIG. 19B). These findings are consistent with the variable presence of insoluble phosphorylated α-synuclein detected via Western blot (FIG. 18B). Notably, while the majority of the impacted cells appeared to be neuronal, there was no overlap with GFAP staining, suggesting there may be colocalization with astrocytes (arrows in FIG. 19B). Together, these findings indicate that when E46K PFFs are forced to use WT α-synuclein as substrate, replication kinetics are delayed.

### E. Discussion

The E46K mutation in α-synuclein was first identified in a Spanish family with autosomal dominant PD and DLB [50]. These patients exhibited early onset of severe parkinsonism and dementia in addition to visual hallucinations. The point mutation is located in the fourth of seven imperfect KTKEGV repeats, which are thought to support α-synuclein's interactions with lipids [5]. Until the recent discoveries of the T72M and E83Q mutations [7, 20], E46K was the only α-synuclein mutation known to disrupt one of the KTKEGV repeats, suggesting it may exert differing effects on α-synuclein biology and pathogenesis than other PD-causing mutations. Studies investigating the effect of the E46K mutation on α-synuclein fibrillization kinetics found that the amino acid substitution increases the protein's propensity to fibrillize compared to WT α-synuclein [14]. Interestingly, this increase in kinetics is not as significant as the effect of the PD-causing A53T mutation. Consistent with these data, disease onset in TgM47^{+/+} mice is delayed compared to the TgM83^{+/+} mouse model [6]. However, both of these models develop a spontaneous synucleinopathy, whereas the TgM20^{+/+} mice—which express WT human α-synuclein—do not.

The ability of MSA α-synuclein prions to propagate in cultured cells was compared using WT, E46K, and A53T α-synuclein as substrate [43]. While the MSA patient samples easily replicated in HEK293T cells expressing WT and A53T α-syn-YFP fusion proteins, the presence of the E46K mutation inhibited in vitro propagation. Cryo-EM structures of misfolded α-synuclein fibrils isolated from MSA patient samples, as well as many recombinant WT fibrils (which misfold into conformations that differ from those seen in MSA patients), contain a Greek key motif that is stabilized by a salt bridge between residues E46 and K80 [16, 24, 25, 28, 34]. It has been hypothesized that the E46K mutation results in a repulsion between the two lysines at residues 46 and 80, which interferes with MSA prion propagation in vitro [19, 34, 43]. Consistent with this hypothesis, cryo-EM structures of recombinant fibrils made using E46K α-synuclein lack the E46/K80 salt bridge; instead, the protein adopts an alternate conformation that is stabilized by a salt bridge between residues K45 and E57 [2]. Together, these structural and biological data suggest that MSA prions cannot use E46K α-synuclein as a substrate for templating. In comparison, the recently resolved structure of α-synuclein fibrils isolated from LBD patients show the conformation is stabilized by a salt bridge between residues E35 and K80, leaving E46 to project outward [48]. While it is not immediately obvious from this structure how the E46K mutation impacts α-synuclein fibrillization in LBD patients, it is clear that the mutation does not disrupt or interfere with protein misfolding.

To test the hypothesis that the E46K mutation inhibits in vivo propagation of MSA prions, TgM47^{+/-} mice—which do not develop spontaneous disease (FIGS. 20A-20C and Table 5)—were inoculated with brain homogenates prepared from deceased MSA patient samples. MSA inoculations induce neurological disease in ˜120 dpi in the TgM83^{+/-} mouse model [31, 41, 44, 47] and in ˜281 dpi in the TgM20^{+/-} model [17]. However, the same MSA samples were unable to transmit disease to the TgM47^{+/-} animals by 475 dpi (FIGS. 14A-14C, Table 10).

TABLE 10
Summary of transmission studies in mice
expressing E46K or WT α-synuclein

| | Mouse Model | | | |
|---|---|---|---|---|
| | | Clinical | Cell | pSer129 |
| Inoculum | Line | Signs | Infection | Neuropathology |
| Control | TgM47^{+/-} | No | No | No |
| | (E46K) | | | |
| Control | TgM20^{+/-} (WT) | No | No | No |
| MSA | TgM47^{+/-} | No | No | No |
| | (E46K) | | | |
| MSA^{a} | TgM20^{+/-} (WT) | Yes | Yes | Yes |
| E46K PFFs | TgM47^{+/-} | Yes | E46K, | Yes |
| | (E46K) | | A53T only | |
| E46K PFFs | TgM20^{+/-} (WT) | 5 of | Yes | Mild |
| | | 8 mice | | |
| WT PFFs | TgM47^{+/-} | No | Yes | No |
| | (E46K) | | | |
| WT PFFs^{a} | TgM20^{+/-} (WT) | Yes | Yes | Yes |

^{a}Data published in Holec et al, 2022 [17], and included here for comparison.

Moreover, the presence of preclinical MSA prion propagation was not detected in the brains of the inoculated mice using multiple assays on either frozen or fixed brain tissue from the animals (FIGS. 14A-14C, Table 6, and FIGS. 21A and B). These findings demonstrate that the in vitro α-syn-YFP cell assays are predictive of in vivo strain biology. Given that the cell assay can be run in 4 days while the animal bioassay can require up to 500 days, more widespread use of these cell lines will accelerate research investigating strain biology. Additionally, these data underscore the conformational, or strain, differences between the α-synuclein prions responsible for disease in MSA patients and those responsible for disease in patients harboring the E46K mutation. These critical differences argue for the need to incorporate strain specificity into synucleinopathy research to better facilitate successful translation of discoveries into the clinic.

Unlike mice inoculated with MSA samples, TgM47^{+/-} mice inoculated with E46K PFFs developed neurological signs, sarkosyl-insoluble hyperphosphorylated aggregates, and α-synuclein neuropathology (FIGS. 15A-16B, Tables 7 and 10). However, the extensive incubation period for these transmissions, compared to studies using the TgM83^{+/-} mice, suggests the E46K mutation exhibits slower misfolding kinetics than the A53T mutation. These findings are consistent with data showing that E46K α-synuclein does not fibrillize as readily as A53T α-synuclein and that disease onset in the TgM83^{+/+} mice occurs earlier than in the TgM47^{+/+} animals [6]. The effect of the relatively slower misfolding kinetics is particularly noticeable in the cell assay data from mice inoculated with E46K PFFs (Table 2). The finding that the α-synuclein prions could only propagate in cells containing both the E46K and A53T mutations is reminiscent of the nucleus size (ns) studies on yeast prions by Tricia Serio's group [40]. This work showed that for yeast strains to propagate, the fibril size must exceed a minimum ns prior to budding, otherwise the fibril becomes unstable and the amyloid disassembles. In this cell model, HEK293T cells double approximately once each day. While strains with faster self-templating kinetics will be able to exceed ns prior to cell division, strains with slower kinetic properties will likely fall below ns and the infection will not be maintained. This may be partially rescued by using an α-syn-YFP construct with mutations that accelerate misfolding kinetics, as was observed here using the E46K,A53T cells.

Notably, the neuropathological inclusions present in E46K PFF-inoculated TgM47^{+/-} mice differed substantially from the pathology previously reported in MSA-inoculated TgM83^{+/-} animals (FIG. 16B). The MSA patient samples induce inclusions throughout neurites in addition to inclusions that fill the entire neuronal cell body. However, most of the pathology seen in TgM47^{+/-} mice inoculated with E46K PFFs can be characterized as round perinuclear inclusions similar in shape to Lewy bodies in PD patients. This is consistent with the conclusion that α-synuclein misfolds into distinct conformations, or strains, in MSA and in PD patients carrying the E46K mutation.

While incompatibility has been shown between MSA prions and E46K α-synuclein in vivo, Long et al. recently reported that fibrils made from recombinant WT α-synuclein are unable to nucleate fibril extension using E46K α-synuclein as substrate [27]. Cryo-EM structures suggest this is also due to the presence of an E46/K80 salt bridge in the WT fibrils, which prevents the E46K protein from adopting the WT fibril conformation. Similarly, Sacino et al. observed that hippocampal inoculations using WT fibrils (made using α-synuclein residues 21-140) into TgM47^{+/-} mice only induced mild, localized neuropathology in the animals, whereas the same inoculation in TgM83^{+/-} mice induced extensive, widespread inclusions 4 mpi [32]. The finding of preclinical α-synuclein prion propagation in TgM47^{+/-} mice inoculated with WT PFFs is consistent with both of these observations (FIGS. 17A-D, Table 8, Table 10, and FIGS. 22A and B). Cryo-EM studies have shown that WT fibrils can adopt a variety of possible conformations, some of which do not contain the E46/K80 salt bridge [15, 16, 24, 25, 28, 52]. Therefore, within a heterogenous population of WT PFFs, there are likely two pools of conformations: one that can use E46K α-synuclein as substrate and one that cannot. Given this mixture, incubation time and α-synuclein prion content in TgM47^{+/-} mice would be expected to reflect the prevalence of each conformational pool. For example, if the pool predominantly contained WT PFFs lacking the E46/K80 salt bridge, disease onset in TgM47^{+/-} mice would be earlier and end-stage animals would contain more α-synuclein prions. However, no neurological signs were observed in inoculated mice, and the detected α-synuclein prions were not hyperphosphorylated, indicating the presence of immature inclusions. Consistent with this interpretation, α-synuclein neuropathology was not found in the inoculated mice. These results suggest that the WT PFFs generated for inoculation contained a small pool of conformations lacking the E46/K80 salt bridge. This is a notable distinction from the MSA inoculation studies where all known α-synuclein fibril structures in MSA patients contain the E46/K80 salt bridge [34]. As a result, MSA prions are unable to propagate using E46K α-synuclein as substrate, indicating that WT PFFs and MSA patient samples are distinct α-synuclein strains.

Finally, TgM20^{+/-} mice were inoculated with E46K PFFs, and a delay was observed in disease onset with an incomplete attack rate (FIGS. 18A-C, Table 10). Notably, two of the healthy mice euthanized at 475 dpi did contain α-synuclein prions in their brains, which is indicative of preclinical prion propagation. Unlike TgM47^{+/-} mice inoculated with E46K PFFs, neuropathological inclusions in the inoculated TgM20^{+/-} mice were rare, limiting the ability to assess their distribution and appearance (FIGS. 19A-B). However, using the cell assay, E46K PFFs passaged in TgM20^{+/-} mice were able to propagate in all α-syn-YFP cells except those expressing the E46K mutation alone (Table 9). These results indicate that the E46K strain replicated with slower kinetics when WT α-synuclein was available as substrate. This is consistent with the lack of PK-resistant α-synuclein in the samples (FIG. 18C), compared to previous findings that E46K PFFs are resistant to 5 μg PK [43]. These findings may shed important light on the process of α-synuclein propagation in patients harboring a heterozygous E46K mutation.

In summary, the presence of the E46K mutation, either in the host or the agent, exerts a selective pressure on α-synuclein prion replication. Most notably, this mutation completely inhibits MSA prions from replicating in vivo, whereas preclinical propagation of WT PFFs in TgM47^{+/-} mice indicates that WT PFFs and MSA patient samples must contain distinct α-synuclein strains. Homotypic seeding (i.e., E46K PFFs inoculated into mice expressing E46K α-synuclein) results in disease transmission. By comparison, heterotypic seeding of the E46K PFFs into mice expressing WT human α-synuclein delays transmission kinetics. While an important caveat to these studies is that differences in inoculum titer may impact incubation period in both transgenic mouse models, these findings contribute to a greater understanding of the strain differences underlying MSA and PD as well as the mechanisms by which selective pressures impact α-synuclein strain propagation.

### F. Materials and Methods

A number of different materials and methods were used in these studies.

### Human Patient Neuropathology

Neuropathology in human tissue samples received from the Parkinson's UK Brain Bank was assessed following bisection of the brain (one hemisphere fixed in 10% buffered formalin and the other hemisphere sliced coronally, photographed on a grid, and rapidly frozen). Fixed tissue blocks from 20 key brain regions were stained with H&E and Luxol fast blue (LFB). To diagnose and stage disease, appropriate blocks were stained with antibodies against α-synuclein, β-amyloid, tau, and p62. An MSA diagnosis was based on α-synuclein inclusions in oligodendrocytes (Alafuzoff et a., Acta Neuropathol. 2009:117:635-52).

Patient samples obtained from the Massachusetts Alzheimer's Disease Research Center (ADRC) Brain Bank were assessed to confirm the diagnosis of MSA. Fresh brains were dissected down the midline with one half fixed in 10% (vol/vol) neutral buffered formalin and coronally sectioned and the other half coronally sectioned before rapid freezing. The fixed tissue was evaluated histologically using a set of blocked regions representative of a variety of neurodegenerative diseases. All blocks were stained with LFB and H&E. Selected blocks were used for immunohistochemical staining for α-synuclein, β-amyloid, and phosphorylated tau. A confirmed MSA diagnosis required the presence of glial cytoplasmic inclusions (Gilman et al. Neurology 2008:71:670-6).

Demographic information about samples used is included in Table 11.

TABLE 11
Patient sample information

| | | Age at | | | |
|---|---|---|---|---|---|
| Patient | Disease | Death | Sex | Brain Region | Brain Bank |
| C9 | None | 62 | M | Putamen | University of |
| | | | | | Miami |
| C17 | None | 65 | M | Putamen | University of |
| | | | | | Miami |
| MSA5 | MSA | 60 | M | Basal ganglia | Parkinson's UK |
| MSA14 | MSA | 76 | M | Basal ganglia | MADRC^{a} |
| MSA17 | MSA | 60 | M | Substantia nigra | MADRC^{a} |

^{a}Massachusetts Alzheimer's Disease Research Center

### Mice

Animals were maintained in an AAALAC-accredited facility in compliance with the 8^{th} edition of the Guide for the Care and Use of Laboratory Animals. All procedures used in this study were approved by the University of California, San Francisco, Institutional Animal Care and Use Committee. All animals were housed under ABSL-2 conditions in an environmentally controlled room (10-15 air changes per hour) at a 22.5° C.±1.4° C. temperature, a 45%+15% relative humidity, and a 12-hour light/dark cycle. Animals had free access to a Tekland diet from Envigo (Indianapolis, IN) and tap water. Mice were group housed unless an animal's health status necessitated individual housing. The B6; C3-Tg (Prnp-SNCA*E46K)47Gia (referred to here as TgM47^{+/-}) and B6;C3-Tg (Prnp-SNCA) 20Vle (referred to here as TgM20^{+/-}) mice were kindly provided by Dr. Benoit Giasson (University of Florida).

### Inoculations

Fresh-frozen human tissue was used to create a 10% (wt/vol) homogenate using calcium- and magnesium-free 1× Dulbecco's phosphate buffered saline (DPBS) using the Omni Tissue Homogenizer (Omni International). Homogenates were diluted to 1% using 5% (wt/vol) bovine serum albumin in 1×DPBS. Recombinant WT and E46K α-synuclein was aggregated in 1×DPBS as previously described [43]. Fibrils were diluted in 1×DPBS to a final concentration of 1 mg/mL.

Prior to inoculation, eight-week-old TgM47^{+/-} and TgM20^{+/-} mice were anesthetized with isoflurane. Freehand inoculations were performed using 30 μL of the 1% brain homogenate or 1 mg/mL fibrils in the thalamus. Following inoculation, all mice were assessed twice each week for the onset of neurological signs based on standard diagnostic criteria for prion disease [3]. Uninoculated mice were euthanized at 545 days of age. Fibril-inoculated TgM47^{+/-} mice were euthanized 531 days postinoculation (dpi) or following the onset of progressive neurological signs. Control- and MSA-inoculated TgM47^{+/-} mice were euthanized at 475 dpi. TgM20^{+/-} mice were euthanized following onset of progressive neurological signs or at 475 dpi. Following euthanasia, the brain was removed and bisected down the midline. The left hemisphere was frozen for reporter cell assay and biochemical analysis, and the right hemisphere was fixed in formalin for neuropathological assessment.

### α-Synuclein Prion Quantification Assay

Frozen brain tissue was used to make a 10% (wt/vol) brain homogenate in calcium- and magnesium-free 1×DPBS using the Omni Tissue Homogenizer with disposable soft tissue tips (Omni International). Aggregated protein was isolated from the homogenates using phosphotungstic acid (PTA: Sigma) as described [33, 47]. Isolated protein pellets were diluted 1:10 in 1×DPBS before testing in the α-synuclein prion quantification assays. HEK293T cells expressing α-syn140-YFP (WT), α-syn140*E46K-YFP (E46K), α-syn140*A53T-YFP (A53T), α-syn140*A30P,A53T-YFP (A30P,A53T), α-syn140*E46K,A53T-YFP (E46K,A53T), and α-syn95*A53T-YFP (1-95) were generated and cultured as previously reported, and assay conditions were used as described [43]. Cells incubated with isolated protein pellets were imaged using the IN Cell Analyzer 6000 (GE Healthcare). DAPI and FITC images from five different regions in each well were analyzed using IN Cell Developer software with an algorithm designed to quantify intracellular aggregates, represented as total fluorescence per cell (×10³; arbitrary units: A.U.). One measurement was generated for each well across the five regions imaged, and each sample was tested in six replicate wells.

### Immunohistochemistry and Neuropathology

Formalin-fixed mouse half-brains were cut into four sections prior to processing through graded alcohols, clearing with xylene, infiltrating with paraffin, and embedding. Thin sections (8 μm) were cut, collected on slides, deparaffinized, and exposed to heat-mediated antigen retrieval with citrate buffer (0.1 M, pH 6) for 20 min. Slides were stained using the Thermo Fisher 480S Autostainer with 30 min blocking in 10% (vol/vol) normal goat serum and incubating with primary and secondary antibodies (2 h each). Primary antibodies used include phosphorylated (S129) α-synuclein (EP1536Y; 1:1,000; Abcam) and glial fibrillary acidic protein (GFAP; 1:500; Abcam). Secondary antibodies conjugated to AlexaFluor 488 or 647 (1:500; Thermo Fisher) were used. Slides were imaged using the Zeiss AxioScan.Z1 and were then analyzed using the ZEN Analysis software package (Zeiss). To quantify α-synuclein neuropathology, a pixel intensity threshold was determined using a positive control slide. Regions of interest were drawn around the caudoputamen, hippocampus, piriform cortex and amygdala, thalamus, hypothalamus, midbrain, and pons. The percentage of pixels positive for staining in each region was determined and averaged across inoculation groups.

### Guanidine Hydrochloride Denaturation

Guanidine hydrochloride (GdnHCl) denaturation of brain homogenates was performed as previously reported [43]. The resulting protein pellets were resuspended in 40 μL 1× NuPAGE LDS loading buffer containing β-mercaptoethanol (20 μL for 5 M samples) and were boiled for 10 min prior to immunoblotting.

### Proteinase K Digestion

Proteinase K (PK) digestion of brain homogenates was performed as previously reported [43]. Final protein pellets were resuspended in 50 μL 1× NuPAGE LDS loading buffer containing β-mercaptoethanol and boiled for 10 min prior to immunoblotting.

### Immunoblotting

To visualize soluble α-synuclein expression in TgM47^{+/-} and TgM20^{+/-} mice, brain homogenates were clarified by centrifugation for 5 min at 1,000×g. The supernatant was collected, and total protein was measured using the bicinchoninic acid (BCA) assay (Pierce). A total of 2.5 μg total protein was prepared in 1× NuPAGE LDS loading buffer and boiled for 10 min. Samples were loaded onto a 10% Bis-Tris gel and SDS-PAGE was performed using the MES buffer system. Protein was transferred to a polyvinylidene fluoride (PVDF) membrane, and the membrane was fixed in 0.4% formalin for 30 min at room temperature. The membrane was then incubated in blocking buffer (5% [wt/vol] nonfat milk in 1× Tris-buffered saline containing 0.05% [vol/vol] Tween 20 [TBST]) for 30 min at room temperature. TgM47^{+/-} blots were incubated with primary antibody for total α-synuclein (MJFR1; 1:12,500; Abcam) and the loading control vinculin (1:10,000: Abcam) in blocking buffer overnight at 4° C. in a vacuum-sealed pouch. TgM20^{+/-} blots were incubated with primary antibody for phosphorylated (S129) α-synuclein (EP1536Y; 1:4,000; Abcam) and the loading control vinculin (1:10,000) in blocking buffer overnight at 4° C. in a vacuum-sealed pouch. Membranes were washed three times with 1×TBST before incubating with horseradish peroxidase-conjugated goat anti-rabbit secondary antibody (1:10,000; Abcam) diluted in blocking buffer for 1 h at 4° C. in a vacuum-sealed pouch. After washing blots three times in 1×TBST, membranes were developed using the enhanced chemiluminescent detection system (Pierce) for exposure to X-ray film.

To detect insoluble phosphorylated α-synuclein, protein aggregates were isolated via PTA precipitation, and resuspended pellets were diluted 1:5 in 1× NuPAGE LDS loading buffer and 1×DPBS. Samples were boiled for 20 min before loading on a 10% Bis-Tris gel. Using the protocol described above, PVDF membranes were probed for phosphorylated (S129) α-synuclein (EP1536Y; 1:4,000). A positive control (MSA-inoculated TgM83^{+/-} mouse sample) was used to show successful protein transfer when warranted. Following guanidine hydrochloride (GdnHCl) denaturation and PK digestion, protein pellets were probed for total α-synuclein using the MJFR1 primary antibody (1:10,000). PK-digested pellets from TgM20^{+/-} mice were probed for phosphorylated (S129) α-synuclein using the EP1536Y primary antibody (1:4,000). For insoluble, GdnHCl, and PK blots from TgM47^{+/-} mice, the membrane was not fixed prior to incubating in blocking buffer.

### Statistical Analysis

Data are presented as mean±standard deviation. Data were analyzed using GraphPad Prism software. Kaplan-Meier curves were analyzed using a log-rank Mantel-Cox test. Analysis of data collected from the α-syn-YFP cell assays and neuropathology data comparing control- and MSA-inoculated samples was performed using a two-way ANOVA with a Tukey multiple comparison post hoc test. Neuropathology data comparing control- and fibril-inoculated mice were analyzed using a two-way ANOVA with a Bonferroni multiple comparison post hoc test. Significance was determined with a P-value <0.05.

### G. References for Example 5

The following list of references refers to the citation numbers of this Example:
1 Appel-Cresswell S, Vilarino-Guell C, Encarnacion M, Sherman H, Yu I, Shah B, et al. (2013) Alpha-synuclein p.H50Q, a novel pathogenic mutation for Parkinson's disease. Mov Disord 28:811-813
2 Boyer D R, Li B, Sun C, Fan W, Zhou K, Hughes M P, et al. (2020) The α-synuclein hereditary mutation E46K unlocks a more stable, pathogenic fibril structure. Proc Natl Acad Sci US A 117:3592-3602
3 Carlson G A, Kingsbury D T, Goodman P A, Coleman S, Marshall S T, DeArmond S, et al. (1986) Linkage of prion protein and scrapie incubation time genes. Cell 46:503-511
4 Daida K, Shimonaka S, Shiba-Fukushima K, Ogata J, Yoshino H, Okuzumi A, et al. (2022) α-Synuclein V15A Variant in Familial Parkinson's Disease Exhibits a Weaker Lipid-Binding Property. Mov Disord 10:2075-2085
5 Davidson W S, Jonas A, Clayton D F, George J M (1998) Stabilization of alpha-synuclein secondary structure upon binding to synthetic membranes. J Biol Chem 273:9443-9449
6 Emmer K L, Waxman E A, Covy J P, Giasson B I (2011) E46K human alpha-synuclein transgenic mice develop Lewy-like and tau pathology associated with age-dependent, detrimental motor impairment. J Biol Chem 286:35104-35118
7 Fevga C, Park Y, Lohmann E, Kievit A J, Breedveld G J, Ferraro F, et al. (2021) A new alpha-synuclein missense variant (Thr72Met) in two Turkish families with Parkinson's disease. Parkinsonism Relat Disord 89:63-72
8 Ghanem S S, Majbour N K, Vaikath N N, Ardah M T, Erskine D, Jensen N M, et al. (2022) α-Synuclein phosphorylation at serine 129 occurs after initial protein deposition and inhibits seeded fibril formation and toxicity. Proc Natl Acad Sci USA 119: e2109617119
9 Giasson B I, Duda J E, Quinn S M, Zhang B, Trojanowski J Q, Lee V M (2002) Neuronal α-synucleinopathy with severe movement disorder in mice expressing A53T human α-synuclein. Neuron 34:521-533
10 Gilman S, Low P A, Quinn N, Albanese A, Ben-Shlomo Y, Fowler C J, et al. (1999) Consensus statement on the diagnosis of multiple system atrophy. J Neurol Sci 163:94-98
11 Gilman S, May S J, Shults C W, Tanner C M, Kukull W, Lee V M-Y, et al. (2005) The North American Multiple System Atrophy Study Group. J Neural Transm (Vienna) 112:1687-1694
12 Gilman S, Wenning G K, Low P A, Brooks D J, Mathias C J, Trojanowski J Q, et al. (2008) Second consensus statement on the diagnosis of multiple system atrophy. Neurology 71:670-676
13 Goldmann W, Hunter N, Smith G, Foster J, Hope J (1994) PrP genotype and agent effects in scrapie: change in allelic interaction with different isolates of agent in sheep, a natural host of scrapie. J Gen Virol 75:989-995
14 Greenbaum E A, Graves C L, Mishizen-Eberz A J, Lupoli M A, Lynch D R, Englander S W, et al. (2005) The E46K mutation in alpha-synuclein increases amyloid fibril formation. J Biol Chem 280:7800-7807
15 Guerrero-Ferreira R, Taylor N M, Arteni A A, Kumari P, Mona D, Ringler P, et al. (2019) Two new polymorphic structures of human full-length alpha-synuclein fibrils solved by cryo-electron microscopy. Elife 8: e48907
16 Guerrero-Ferreira R, Taylor N M, Mona D, Ringler P, Lauer M E, Riek R, et al. (2018) Cryo-E M structure of alpha-synuclein fibrils. eLife 7: e36402
17 Holec S A M, Lee J, Oehler A, Ooi F K, Mordes D A, Olson S H, et al. (2022) Multiple system atrophy prions transmit neurological disease to mice expressing wild-type human α-synuclein. Acta Neuropathol 144:677-690
18 Holec S A M, Liu S L, Woerman A L (2022) Consequences of variability in α-synuclein fibril structure on strain biology. Acta Neuropathol 143:311-330
19 Holec S A M, Woerman A L (2021) Evidence of distinct α-synuclein strains underlying disease heterogeneity. Acta Neuropathol 142:73-86
20 Kapasi A, Brosch J R, Nudelman K N, Agrawal S, Foroud T M, Schneider J A (2020) A novel SNCA E83Q mutation in a case of dementia with Lewy bodies and atypical frontotemporal lobar degeneration. Neuropathology 40:620-626
21 Kiely A P, Asi Y T, Kara E, Limousin P, Ling H, Lewis P, et al. (2013) α-Synucleinopathy associated with G51D SNCA mutation: a link between Parkinson's disease and multiple system atrophy? Acta Neuropathol 125:753-769
22 Krüger R, Kuhn W, Müller T, Woitalla D, Graeber M, Kösel S, et al. (1998) Ala30Pro mutation in the gene encoding alpha-synuclein in Parkinson's disease. Nat Genet 18:106-108
23 Lesage S, Anheim M, Letournel F, Bousset L, Honoré A, Rozas N, et al. (2013) G51D α-synuclein mutation causes a novel parkinsonian-pyramidal syndrome. Ann Neurol 73:459-471
24 Li B, Ge P, Murray K A, Sheth P, Zhang M, Nair G, et al. (2018) Cryo-E M of full-length α-synuclein reveals fibril polymorphs with a common structural kernel. Nat Commun 9:3609
25 Li Y, Zhao C, Luo F, Liu Z, Gui X, Luo Z, et al. (2018) Amyloid fibril structure of α-synuclein determined by cryo-electron microscopy. Cell Res 28:897-903
26 Liu H, Koros C, Strohäker T, Schulte C, Bozi M, Varvaresos S, et al. (2021) A Novel SNCA A30G Mutation Causes Familial Parkinson's Disease. Mov Disord 36:1624-1633
27 Long H, Zheng W, Liu Y, Sun Y, Zhao K, Liu Z, et al. (2021) Wild-type α-synuclein inherits the structure and exacerbated neuropathology of E46K mutant fibril strain by cross-seeding. Proc Natl Acad Sci USA 118: e2012435118
28 Ni X, McGlinchey R P, Jiang J, Lee J C (2019) Structural Insights into α-Synuclein Fibril Polymorphism: Effects of Parkinson's Disease-Related C-Terminal Truncations. J Mol Biol 431:3913-3919
29 Pasanen P, Myllykangas L, Siitonen M, Raunio A, Kaakkola S, Lyytinen J, et al. (2014) A novel α-synuclein mutation A53E associated with atypical multiple system atrophy and Parkinson's disease-type pathology. Neurobiol Aging 35:2180.e1-5
30 Polymeropoulos M H, Lavedan C, Leroy E, Ide S E, Dehejia A, Dutra A, et al. (1997) Mutation in the α-synuclein gene identified in families with Parkinson's disease. Science 276:2045-2047
31 Prusiner S B, Woerman A L, Mordes D A, Watts J C, Rampersaud R, Berry D B, et al. (2015) Evidence for α-synuclein prions causing multiple system atrophy in humans with parkinsonism. Proc Natl Acad Sci USA 112: E5308-E5317
32 Sacino A N, Brooks M, Thomas M A, Mckinney A B, McGarvey N H, Rutherford N J, et al. (2014) Amyloidogenic alpha-synuclein seeds do not invariably induce rapid, widespread pathology in mice. Acta Neuropathol 127:645-665
33 Safar J, Wille H, Itri V, Groth D, Serban H, Torchia M, et al. (1998) Eight prion strains have PrPSc molecules with different conformations. Nat Med 4:1157-1165
34 Schweighauser M, Shi Y, Tarutani A, Kametani F, Murzin A G, Ghetti B, et al. (2020) Structures of α-synuclein filaments from multiple system atrophy. Nature 585:464-469
35 Shibuya S, Higuchi J, Shin R-W, Tateishi J, Kitamoto T (1998) Protective prion protein polymorphisms against sporadic Creutzfeldt-Jakob disease. Lancet 351:419
36 Spillantini M G, Schmidt M L, Lee V M-Y, Trojanowski J Q, Jakes R, Goedert M (1997) α-Synuclein in Lewy bodies. Nature 388:839-840
37 Stewart P, Campbell L, Skogtvedt S, Griffin K A, Arnemo J M, Tryland M, et al. (2012) Genetic predictions of prion disease susceptibility in carnivore species based on variability of the prion gene coding region. PLOS One 7: e50623
38 Thomzig A, Wagenführ K, Pinder P, Joncic M, Schulz-Schaeffer W J, Beekes M (2021) Transmissible α-synuclein seeding activity in brain and stomach of patients with Parkinson's disease. Acta Neuropathol 141:861-879
39 Tuttle M D, Comellas G, Nieuwkoop A J, Covell D J, Berthold D A, Kloepper K D, et al. (2016) Solid-state NMR structure of a pathogenic fibril of full-length human alpha-synuclein. Nat Struct Mol Biol 23:409-415
40 Villali J, Dark J, Brechtel T M, Pei F, Sindi S S, Serio T R (2020) Nucleation seed size determines amyloid clearance and establishes a barrier to prion appearance in yeast. Nat Struct Mol Biol 27:540-549
41 Watts J C, Giles K, Oehler A, Middleton L, Dexter D T, Gentleman S M, et al. (2013) Transmission of multiple system atrophy prions to transgenic mice. Proc Natl Acad Sci USA 110:19555-19560
42 Westaway D, Zuliani V, Cooper C M, Da Costa M, Neuman S, Jenny A L, et al. (1994) Homozygosity for prion protein alleles encoding glutamine-171 renders sheep susceptible to natural scrapie. Genes Dev 8:959-969
43 Woerman A L, Kazmi S A, Patel S, Aoyagi A, Oehler A, Widjaja K, et al. (2018) Familial Parkinson's point mutation abolishes multiple system atrophy prion replication. Proc Natl Acad Sci USA 115:409-414
44 Woerman A L, Kazmi S A, Patel S, Freyman Y, Oehler A, Aoyagi A, et al. (2018) MSA prions exhibit remarkable stability and resistance to inactivation. Acta Neuropathol 135:49-63
45 Woerman A L, Patel S, Kazmi S A, Oehler A, Freyman Y, Espiritu L, et al. (2017) Kinetics of human mutant tau prion formation in the brains of two transgenic mouse lines. JAMA Neurol 74:1464-1472
46 Woerman A L, Patel S, Kazmi S A, Oehler A, Lee J, Mordes D A, et al. (2020) Kinetics of α-synuclein prions preceding neuropathological inclusions in multiple system atrophy. PLOS Pathog 16: e1008222
47 Woerman A L, Stöhr J, Aoyagi A, Rampersaud R, Krejciova Z, Watts J C, et al. (2015) Propagation of prions causing synucleinopathies in cultured cells. Proc Natl Acad Sci USA 112: E4949-E4958
48 Yang Y, Shi Y, Schweighauser M, Zhang X, Kotecha A, Murzin A G, et al. (2022) Structures of α-synuclein filaments from human brains with Lewy pathology. Nature 610:791-795
49 Yoshino H, Hirano M, Stoessl A J, Imamichi Y, Ikeda A, Li Y, et al. (2017) Homozygous alpha-synuclein p.A53V in familial Parkinson's disease. Neurobiol Aging 57:248. e7-248.e12
50 Zarranz J J, Alegre J, Gómez-Esteban J C, Lezcano E, Ros R, Ampuero I, et al. (2004) The new mutation, E46K, of α-synuclein causes Parkinson and Lewy body dementia. Ann Neurol 55:164-173
51 Zhao K, Li Y, Liu Z, Long H, Zhao C, Luo F, et al. (2020) Parkinson's disease associated mutation E46K of α-synuclein triggers the formation of a distinct fibril structure. Nat Commun 11:2643
52 Zhao K, Lim Y J, Liu Z, Long H, Sun Y, Hu J J, et al. (2020) Parkinson's disease-related phosphorylation at Tyr39 rearranges α-synuclein amyloid fibril structure revealed by cryo-E M. Proc Natl Acad Sci USA 117:20305-20315

### Example 5. Example 6. Studies on Additional α-Synuclein Mutations

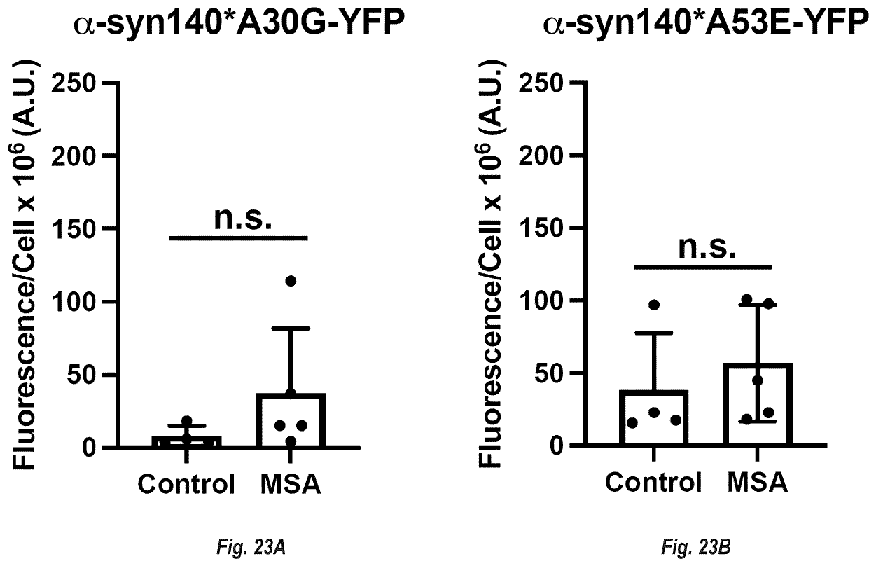

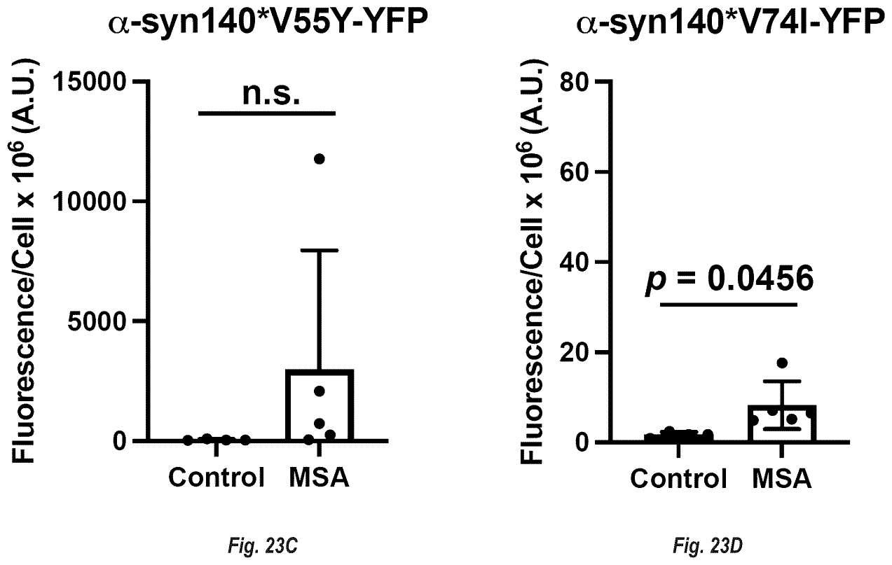

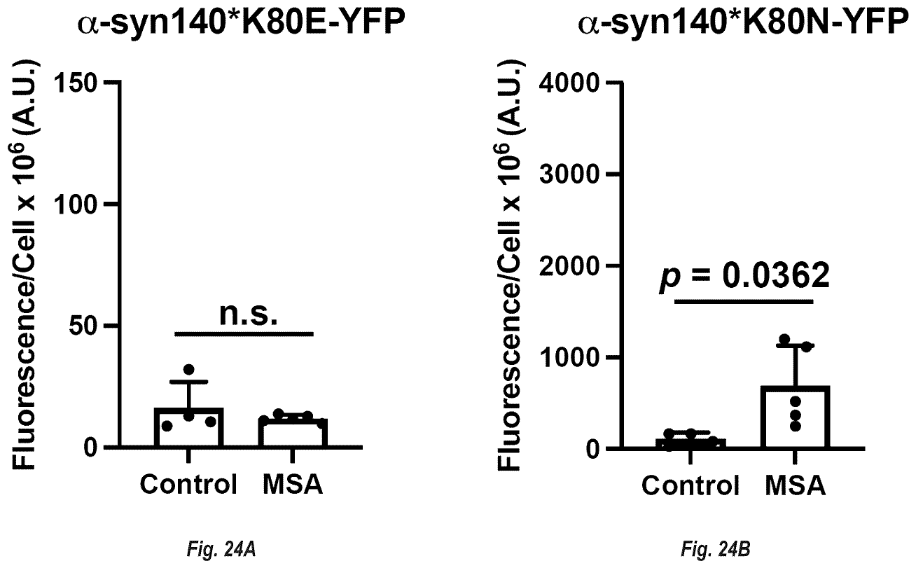

Additional control and MSA patient samples were tested for their ability to infect or replicate in α-syn-YFP cell lines. The familial mutations A30G (FIG. 23A) and A53E (FIG. 23B) blocked MSA replication (p>0.05). Additionally, the novel mutations V55Y (FIG. 23C) and V74I (FIG. 23D) also prevented MSA infection (V55Y, p>0.05; V74I, p=0.0456).

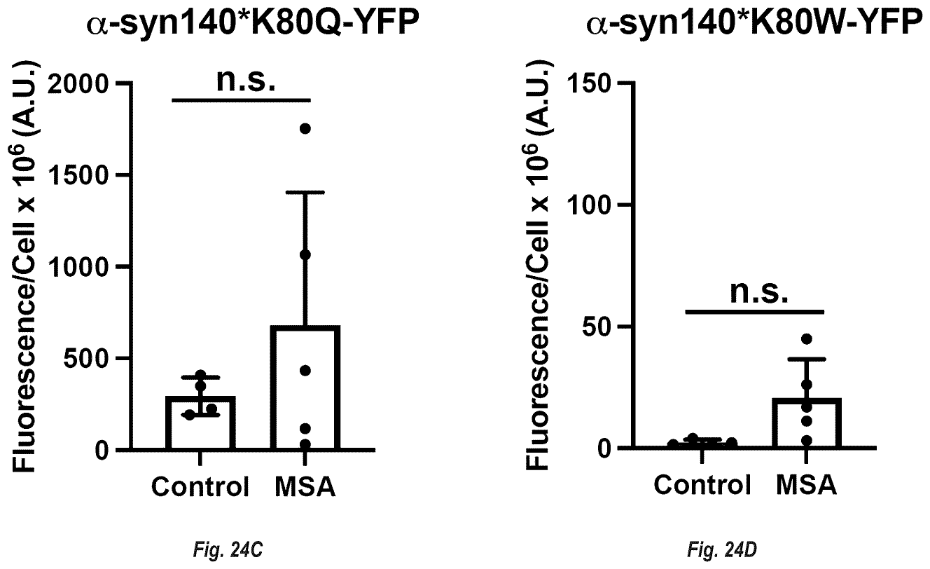

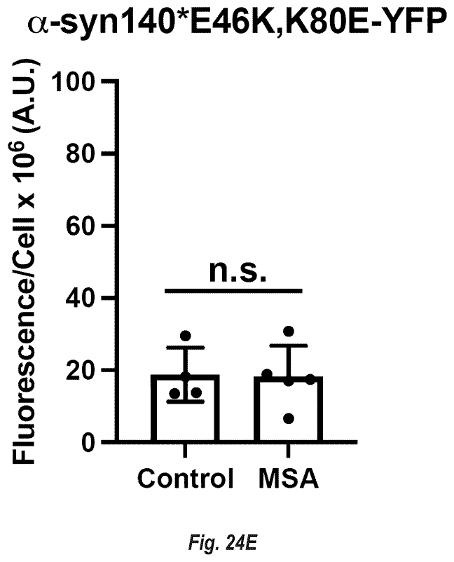

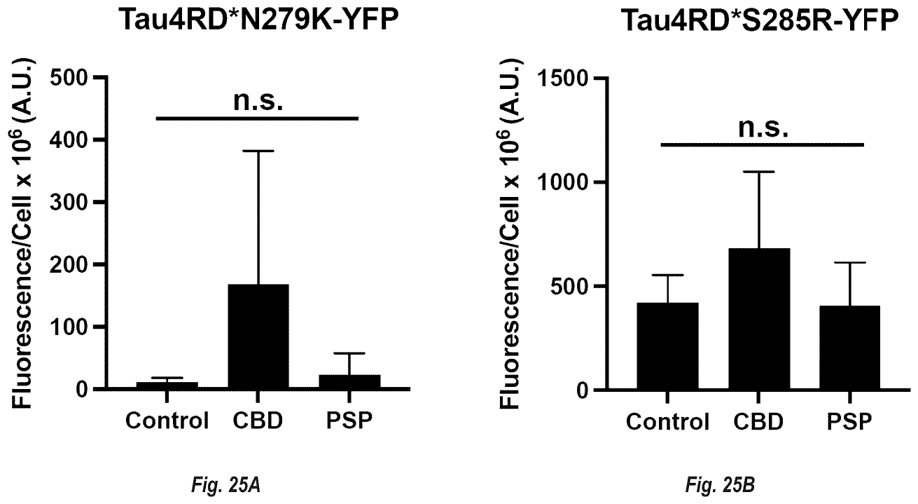

Four control and five MSA patient samples were tested for their ability to infect cells expressing novel mutations at residue K80 with the E/N/Q/W substitution (FIGS. 24A-24D). All four mutations significantly interfered with MSA replication, with the strongest effects due to the K80E and K80W mutations (p>0.05).

The E46K mutation has previously been reported to block MSA replication in vitro. Hypothesizing that the E46 and K80 mutations inhibit MSA replication by disrupting a salt bridge that stabilizes the misfolded conformation between the two residues, residue swapping was tested for the ability to restore protein misfolding. Cells expressing the double E46K,K80E mutation (FIG. 24E) were also resistant to MSA replication (p>0.05).

### Example 7. Studies on Additional Tau Mutations

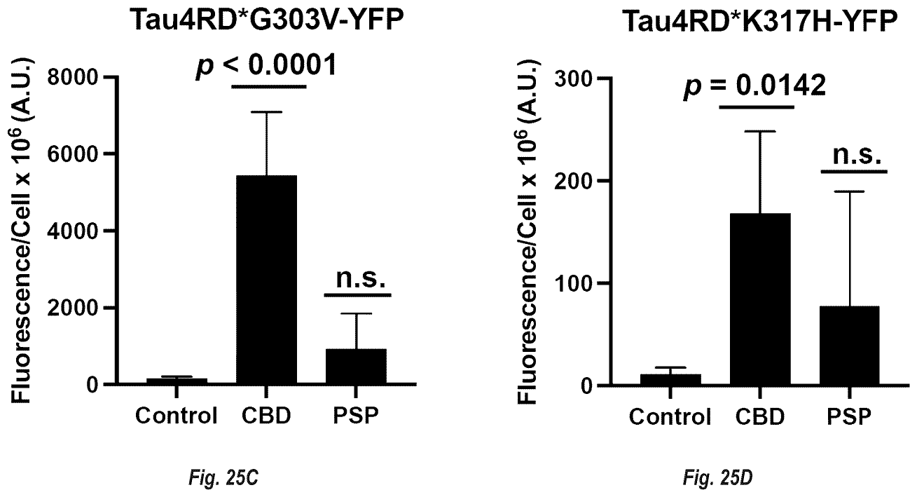

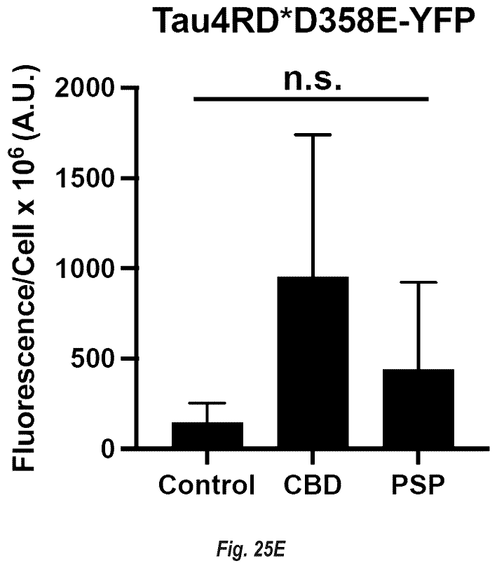

Corticobasal degeneration (CBD) and progressive supranuclear palsy (PSP) patient samples were tested for their ability to infect or replicate in HEK293T cells expressing the Tau4RD construct fused to YFP. The N279K (FIG. 25A), G303V (FIG. 25C), K317H (FIG. 25D), and D358E mutations (FIG. 25E) inhibited or reduced PSP replication with little to no effect on the CBD patient samples. The S285R mutation (FIG. 25B) interfered with both CBD and PSP replication.

### EQUIVALENTS

The foregoing written specification is considered to be sufficient to enable one skilled in the art to practice the embodiments. The foregoing description and Examples detail certain embodiments and describe the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5 of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

What is claimed is:

1. An α-syn mutant nucleic acid encoding for a α-syn mutant protein comprising one or more amino acid substitutions as compared to SEQ ID NO: 2 chosen from:
(a) a mutation of Q79, S87, T81, Q24, T64, N65, T75, T92, and/or T81 to alanine, valine, isoleucine, leucine, methionine, glycine, proline, or phenylalanine;
(b) a mutation of G25, G36, G41, G47, G51, G67, G73, G84, G86, and/or G93 to proline, valine, isoleucine, methionine, threonine, or tryptophan;
(c) a mutation of T22, G31, G36, G68, T72, G73, E83, G84, and/or D98 to cysteine, alanine, glutamic acid, glutamine, isoleucine, leucine, lysine, histidine, tryptophan, valine, or tyrosine;
(d) a mutation of H50, K45 and/or K47 to a hydrophobic and/or nonchelating amino acid such as alanine, cysteine, phenylalanine, glycine, isoleucine, lysine, leucine, methionine, asparagine, proline, glutamine, serine, threonine, valine, tryptophan, or tyrosine;
(e) a mutation of T59, T54 and/or T72 to a nonpolar amino acid, an acidic amino acid, or a basic amino acid such as alanine, cysteine, phenylalanine, glycine, isoleucine, lysine, leucine, methionine, asparagine, proline, glutamine, valine, tryptophan, or tyrosine; and/or
(f) an A30G, A53E, E46K, V55Y, A53E, T72M, V74I, and/or K80E, K80N, or K80W mutation.

2. The α-syn mutant nucleic acid of claim 1, wherein;
a. the substitution is K80E, K80N, or K80W;
b. the nucleic acid comprises two or more, three or more, four or more, or five or more amino acid substitutions as compared to SEQ ID NO: 2; and/or
c. the two or more amino acid substitutions comprise E46K and K80E.

3. A tau mutant nucleic acid encoding for a tau mutant protein comprising one or more amino acid substitutions as compared to SEQ ID NO: 4 chosen from:
(a) a G303V, K317H, D358E, N279K, G367M/V, G323F, G334F/W/A/I, G335A/I, G333A/I, G366A/I, and/or S285R mutation;
(b) a mutation of G272, K274, V275, I277, K298, G304, S305, V306, V309, K317, N327, I328, D348, P364, N368, T377, F378, R379, E380, H388, and/or E391 to a rigid amino acids such as proline, valine, isoleucine, methionine, threonine, or tryptophan;
(c) a mutation of Q269, K274, V275, I277, K280, K281, L284, N286, Q288, K290, S293, K294, D295, N296, K298, H299, S305, V306, Q307, 1308, V309, K311, L315, K317, C322, S324, L325, N327, I328, H329, K331, E338, K343, D345, D348, R349, L357, D358, N359, 1360, V363, N368, K370, H374, L376, R379, E380, N381, H388, E391, K395, H407, D421, A426, D430, E431, and/or A434 to amino acids to proline, valine, isoleucine, methionine, threonine or tryptophan; and/or
(d) a mutation of G271, G272, G273, K274, I277, K281, D283, S285, N286, Q288, S289, G292, N296, K298, G302, G303, G304, S305, Y310, K317, S320, C322, G323, S324, L325, G326, I328, H330, P332, G333, G334, G335, E338, S341, E342, K347, D348, S352, I354, G355, S356, D358, H362, P364, G365, G366, G367, T377, F378, R379, E380, N381, H388, G401, A426, and/or G440 to cysteine, alanine, glutamic acid, glutamine, isoleucine, leucine, lysine, histidine, tryptophan, valine, or tyrosine.

4. The tau mutant nucleic acid of claim 3, wherein the substitution is chosen from K317H, D358E, N279K and/or S285R and/or the nucleic acid comprises two or more, three or more, four or more, or five or more amino acid substitutions as compared to SEQ ID NO: 4.

5. A mutant nucleic acid encoding for a mutant protein comprising one or more amino acid substitutions, wherein the mutant protein is:
(a) prion protein (PrP), optionally wherein the substitution is comprised within the region of amino acids 127-219 of SEQ ID NO: 6;
(b) β-amyloid, optionally wherein the substitution is comprised within amino acids 9-42;
(c) TDP-43;
(d) SOD1;
(e) HTT;
(f) ApoE; or
(g) TMEM106B, optionally wherein the substitution is comprised within amino acids 120-254.

6. A cell comprising a nucleic acid, a mutant protein encoded by a nucleic acid, or a cell comprising a nucleic acid that expresses a mutant protein encoded by a nucleic acid, wherein said nucleic acid is the α-syn mutant nucleic acid of claim 1.

7. A method to inhibit prion propagation of misfolded protein conformations comprising introducing one or more substitutions in the amino acid sequence of the protein so as to inhibit protein misfolding and aggregate formation.

8. A method to treat a neurodegenerative disease, comprising administering an exogenous mutant protein to a subject in need thereof, wherein the neurodegenerative disease is the result of protein misfolding and aggregate formation, wherein the exogenous mutant protein comprises one or more substitutions in the amino acid sequence of the protein so as to inhibit said protein from misfolding.

9. A method to treat a neurodegenerative disease, comprising introducing a mutation in vivo in the gene of the protein that is misfolding in a subject in need thereof, wherein the neurodegenerative disease is the result of protein misfolding and aggregate formation, wherein the mutation results in one or more substitutions in the amino acid sequence of the protein to inhibit said protein from misfolding.

10. The method of claim 7, wherein;
a. the substitution creates a change in the charge of the protein, substitutes a larger or smaller amino acid as compared to the wild type amino acid in the same position, disrupts a salt bridge, changes conformation and/or disrupts zipper formation;
b. the substitution is the result of substituting one amino acid for a larger amino acid;
c. the mutation is introduced by gene therapy or gene editing;
d. the protein is α-syn, tau, prion protein, β-amyloid, TDP-43, SOD1, HTT, ApoE, or TMEM106B;
e. disease symptoms become less severe as the subject expresses more protein from the gene comprising the mutation; and/or
f. the neurodegenerative disease is a:
i. tau-related disease, optionally wherein the tau-related disease is Alzheimer's disease, progressive supranuclear palsy, chronic traumatic encephalopathy, Pick's disease, argyrophilic grain disease, or corticobasal degeneration;
ii. α-synuclein-related disease, optionally wherein the α-synuclein-related disease is Parkinson's disease, multiple system atrophy (MSA), Lewy body diseases, and Parkinson's disease with dementia; Huntington's disease, amyotrophic lateral sclerosis, or diabetes; or
iii. prion protein (PrP)-related disease, optionally wherein the PrP-related disease is Creutzfeldt-Jakob disease, fatal familial insomnia, or Gerstmann-Sträussler-Scheinker disease.

11. The method of claim 10, wherein the protein is α-syn mutant comprising one or more amino acid substitutions as compared to SEQ ID NO: 2 chosen from:
(a) a mutation of Q79, S87, T81, Q24, T64, N65, T75, T92, and/or T81 to alanine, valine, isoleucine, leucine, methionine, glycine, proline, or phenylalanine;
(b) a mutation of G25, G36, G41, G47, G51, G67, G73, G84, G86, and/or G93 to proline, valine, isoleucine, methionine, threonine, or tryptophan;
(c) a mutation of T22, G31, G36, G68, T72, G73, E83, G84, and/or D98 to cysteine, alanine, glutamic acid, glutamine, isoleucine, leucine, lysine, histidine, tryptophan, valine, or tyrosine;
(d) a mutation of H50, K45 and/or K47 to a hydrophobic and/or nonchelating amino acid such as alanine, cysteine, phenylalanine, glycine, isoleucine, lysine, leucine, methionine, asparagine, proline, glutamine, serine, threonine, valine, tryptophan, or tyrosine;
(e) a mutation of T59, T54 and/or T72 to a nonpolar amino acid, an acidic amino acid, or a basic amino acid such as alanine, cysteine, phenylalanine, glycine, isoleucine, lysine, leucine, methionine, asparagine, proline, glutamine, valine, tryptophan, or tyrosine; and/or
(f) an A30G, A53E, E46K, V55Y, A53E, T72M, V74I, and/or K80E, K80N, or K80W mutation.

12. The method of claim 11, wherein;
a. the substitution is K80E, K80N, or K80W;
b. the nucleic acid comprises two or more, three or more, four or more, or five or more amino acid substitutions as compared to SEQ ID NO: 2; and/or
c. the two or more amino acid substitutions comprise E46K and K80E.

13. The method of claim 10, wherein the protein is a tau mutant comprising one or more amino acid substitutions as compared to SEQ ID NO: 4 chosen from:
(a) a G303V, K317H, D358E, N279K, G367M/V, G323F, G334F/W/A/I, G335A/I, G333A/I, G366A/I, and/or S285R mutation;
(b) a mutation of G272, K274, V275, I277, K298, G304, S305, V306, V309, K317, N327, I328, D348, P364, N368, T377, F378, R379, E380, H388, and/or E391 to a rigid amino acids such as proline, valine, isoleucine, methionine, threonine, or tryptophan;
(c) a mutation of Q269, K274, V275, I277, K280, K281, L284, N286, Q288, K290, S293, K294, D295, N296, K298, H299, S305, V306, Q307, 1308, V309, K311, L315, K317, C322, S324, L325, N327, I328, H329, K331, E338, K343, D345, D348, R349, L357, D358, N359, 1360, V363, N368, K370, H374, L376, R379, E380, N381, H388, E391, K395, H407, D421, A426, D430, E431, and/or A434 to amino acids to proline, valine, isoleucine, methionine, threonine or tryptophan; and/or
(d) a mutation of G271, G272, G273, K274, I277, K281, D283, S285, N286, Q288, S289, G292, N296, K298, G302, G303, G304, S305, Y310, K317, S320, C322, G323, S324, L325, G326, I328, H330, P332, G333, G334, G335, E338, S341, E342, K347, D348, S352, I354, G355, S356, D358, H362, P364, G365, G366, G367, T377, F378, R379, E380, N381, H388, G401, A426, and/or G440 to cysteine, alanine, glutamic acid, glutamine, isoleucine, leucine, lysine, histidine, tryptophan, valine, or tyrosine.

14. The method of claim 13;
a. the substitution is chosen from K317H, D358E, N279K and/or S285R; and/or
b. the nucleic acid comprises two or more, three or more, four or more, or five or more amino acid substitutions as compared to SEQ ID NO: 4.

15. A method to treat a neurodegenerative disease, comprising introducing a mutation in vivo in the gene of a protein that is misfolding in a subject in need thereof or administering an exogenous mutant protein comprising a mutation to a subject in need thereof, wherein the neurodegenerative disease is the result of protein misfolding and aggregate formation, wherein the mutation renders the subject functionally heterozygous expressing two variants of the same protein.

16. The method of claim 15, wherein the mutation is introduced by gene therapy or gene editing and/or the protein is PrP, optionally wherein the sequence of PrP comprises SEQ ID NO: 6.

17. The method of claim 15, wherein:
(a) the subject expresses E219, and the mutation is E219K;
(b) the subject expresses K219, and the mutation is K219E;
(c) the subject expresses M129, and the mutation is M129V;
(d) the subject expresses V129, and the mutation is V129M;
(e) the subject expresses V127, and the mutation is V127G; or
(f) the subject expresses G127, and the mutation is G127V.

18. A method to diagnose a neurodegenerative disease comprising:
(a) adding a sample of misfolded protein aggregate from a patient to a panel of cell lines, wherein each cell line expresses a tagged protein of interest comprising a reporter, wherein the protein or proteins of interest contains one or more mutations; and
(b) measuring the amount of aggregation of the reporter in at least one cell line comprised in the panel, wherein the tagged protein of interest expressed in each cell line that lacks aggregation of the reporter indicates the neurodegenerative disease of the patient.

19. The method of claim 18, wherein:
(a) the protein of interest comprised in one or more cell line in the panel is a mutant protein that inhibits aggregation of a protein associated with one or more neurodegenerative disease;
(b) the protein of interest comprised in one or more cell line in the panel is a protein associated with one or more neurodegenerative disease; and/or
(c) the method identifies the misfolding or protein aggregation underlying the subject's symptoms and/or identifies how this misfolding or protein aggregation can be inhibited.

20. The method of claim 18, wherein;
a. the tagged protein of interest expressed in multiple cell lines in the panel that lack aggregation of the reporter indicates the neurodegenerative disease of the patient;
b. one or more cell line in the panel of cell lines is a transformed cell line, optionally wherein the transformed cell line is chosen from NIH 3T3, HeLa, PC12, SH-SY5Y, or HEK293T cells;
c. the sample is obtained from a biopsy, nasal swab, skin, cerebral spinal fluid, and/or plasma samples;
d. the reporter is a fluorescent protein; and/or
e. the method comprises treating said patient for said neurological disorder using the method of claim 12.
